SEQUENCE LISTING (120 sequences)

SEQ ID NO: 1 - DNA, 722 bp, Homo sapiens
cttgttcgtctcactggtgtgagctccagcatcccctttgctcgaaatggaccccaactgctcttgcgccactggtggctcctgcacgtgcgccggctcctgcaagtgcaaagagtgcaaatgcacctcctgcaagaagagtgagtgcggggccatctccaggaatctggggctgtggctcaggttgggagggaactcaaggctggccctgagtgcatccttctggggaactgggctttctttgccctcattgcccgtgtcattccctctccaggctttctgccctaaattcagatggggcaggacagcatttttctcgtgggacacaaaccccaactgtaccccctatggtttcagaacagagctgtgccagacgaaaaaaagcatcctctgggtctgggttctgagctcgagccaggcttgctattagggcagggaggtgcccggtcaagtctactgccacctctcactctccccttcttccccaggctgctgttcctgctgccccgtgggctgtgccaagtgtgcccagggctgcgtctgcaaaggggcatcggagaagtgcagctgctgtgcctgatgtgggaacagctcttctcccagatgtaaatagaacaacctgcacaacctggatttttttaaaaatacaacactgagccatttgctgcatttctttttatactaaatatgtgactgacaataaaaacaattttgactttaaa

SEQ ID NO: 2 - DNA, 384 bp, Homo sapiens
atggaccccaactgctcttgcgccactggtggctcctgcacgtgcgccggctcctgcaagtgcaaagagtgcaaatgcacctcctgcaagaagagtgagtgcggggccatctccaggaatctggggctgtggctcaggttgggagggaactcaaggctggccctgagtgcatccttctggggaactgggctttctttgccctcattgcccgtgtcattccctctccaggctttctgccctaaattcagatggggcaggacagcatttttctcgtgggacacaaaccccaactgtaccccctatggtttcagaacagagctgtgccagacgaaaaaaagcatcctctgggtctgggttctgagctcgagccaggcttgctattag

SEQ ID NO: 3 - protein, 127 aa, Homo sapiens
MDPNCSCATGGSCTCAGSCKCKECKCTSCKKSECGAISRNLGLWLRLGGNSRLALSASFWGTGLSLPSLPVSFPLQAFCPKFRWGRTAFFSWDTNPNCTPYGFRTELCQTKKSILWVWVLSSSQACY

SEQ ID NO: 4 - DNA, 468 bp, Homo sapiens
gccccctcccctgactatcaaagcagcggccggctgttggggtccaccacgccttccacctgccccactgcttcttcgcttctctcttggaaagtccagtctctcctcggcttgcaatggaccccaactgctcctgcgccgctggtgtctcctgcacctgcgctggttcctgcaagtgcaaagagtgcaaatgcacctcctgcaagaagagctgctgctcctgctgccccgtgggctgtagcaagtgtgcccagggctgtgtttgcaaaggggcgtcagagaagtgcagctgctgcgactgatgccaggacaacctttctcccagatgtaaacagagagacatgtacaaacctggatttttttttataccaccttgacccatttgctacattccttttcctgtgaaatatgtgagtgataattaaacactttagacccaaaaaaaaaaaaaaaaaaaaaaaaaaaaaa

SEQ ID NO: 5 - DNA, 186 bp, Homo sapiens
atggaccccaactgctcctgcgccgctggtgtctcctgcacctgcgctggttcctgcaagtgcaaagagtgcaaatgcacctcctgcaagaagagctgctgctcctgctgccccgtgggctgtagcaagtgtgcccagggctgtgtttgcaaaggggcgtcagagaagtgcagctgctgcgactga

SEQ ID NO: 6 - protein, 61 aa, Homo sapiens
MDPNCSCAAGVSCTCAGSCKCKECKCTSCKKSCCSCCPVGCSKCAQGCVCKGASEKCSCCD

SEQ ID NO: 7 - DNA, 367 bp, Homo sapiens
ctccagtctcacctcggcttgcaatggaccccaactgctcctgcgaggctggtggctcctgcgcctgcgccggctcctgcaagtgcaaaaagtgcaaatgcacctcctgcaagaagagctgctgctcctgttgccccctgggctgtgccaagtgtgcccagggctgcatctgcaaaggggcgtcagagaagtgcagctgctgtgcctgatgtcgggacagccctgctgtcagatgaaaacagaatgacacgtaaaatccgaggttttttttttctacaactccgactcatttgctacattcctttttttctgtgaaatatgtgaataataattaaacacttagacttgaaaaaaaaaaaaaaaaaaa

SEQ ID NO: 8 - DNA, 186 bp, Homo sapiens
atggaccccaactgctcctgcgaggctggtggctcctgcgcctgcgccggctcctgcaagtgcaaaaagtgcaaatgcacctcctgcaagaagagctgctgctcctgttgccccctgggctgtgccaagtgtgcccagggctgcatctgcaaaggggcgtcagagaagtgcagctgctgtgcctga

SEQ ID NO: 9 - protein, 61 aa, Homo sapiens
MDPNCSCEAGGSCACAGSCKCKKCKCTSCKKSCCSCCPLGCAKCAQGCICKGASEKCSCCA

SEQ ID NO: 10 - DNA, 396 bp, Homo sapiens
actccgccttccacgtgcacccactgcctcttcccttctcgcttgggaactctagtctcgcctcgggttgcaatggaccccaactgctcctgtgccgctggtgtctcctgcacctgcgccagctcctgcaagtgcaaagagtgcaaatgcacctcctgcaagaagagctgctgctcctgctgccctgtgggctgtgccaagtgtgcccaaggctgcatctgcaaaggggcatcggagaagtgcagctgctgcgcctgatgtcgggacagccctgctcccaagtacaaatagagtgacccgtaaaatctaggattttttgttttttgctacaatcttgacccctttgctacattcccttttttctgtgaaatatgtgaataataattaaacacttag

SEQ ID NO: 11 - DNA, 186 bp, Homo sapiens
atggaccccaactgctcctgtgccgctggtgtctcctgcacctgcgccagctcctgcaagtgcaaagagtgcaaatgcacctcctgcaagaagagctgctgctcctgctgccctgtgggctgtgccaagtgtgcccaaggctgcatctgcaaaggggcatcggagaagtgcagctgctgcgcctga

SEQ ID NO: 12 - protein, 61 aa, Homo sapiens
MDPNCSCAAGVSCTCASSCKCKECKCTSCKKSCCSCCPVGCAKCAQGCICKGASEKCSCCA

SEQ ID NO: 13 - DNA, 454 bp, Homo sapiens
tctgtcccgctgcgtgttttcctcttgatcgggaactcctgcttctccttgcctcgaaatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgcctgatgccaggacagctgtgctctcagatgtaaatagagcaacctatataaacctggatttttttttttttttttttgtacaaccctgacccgtttgctacatctttttttctatgaaatatgtgaatggcaataaattcatctagactaaaaaaaaaaaaaaaaaaaaaaaaaaaaaaaaaaaaaaaaaaaaaaaaaaaaaaaaaaaaaaaaa

SEQ ID NO: 14 - DNA, 186 bp, Homo sapiens
atggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaagggacgtcagacaagtgcagctgctgtgcctga

SEQ ID NO: 15 - protein, 61 aa, Homo sapiens
MDPNCSCSPVGSCACAGSCKCKECKCTSCKKSCCSCCPVGCAKCAQGCICKGTSDKCSCCA

SEQ ID NO: 16 - DNA, 431 bp, Homo sapiens
ccacgccgtccgggtgggcctagcagtcgctccatttatcgcttgagatctccagccttaccgcggctcgaaatggaccccaactgctcctgcaccactggtgtctcctgcgcctgcaccggctcctgcaagtgcaaagagtgcaaatgcacctcctgcaagaagagctgctgctcctgctgccccgtgggctgtgccaagtgtgcccacggctgtgtctgcaaagggacgttggagaactgcagctgctgtgcctgatgtgggaacagctcttctcccagatgttaatagaacaagctgcacaacctggattttttttcaatacgatactgagccatttgctgcatttctttttatgttaaatatgtgagtgacaataaaacaattttgacttgaaaaaaaaaaaaaaaaaaaaaaaaaaaaaaaaaaaa

SEQ ID NO: 17 - DNA, 186 bp, Homo sapiens
atggaccccaactgctcctgcaccactggtgtctcctgcgcctgcaccggctcctgcaagtgcaaagagtgcaaatgcacctcctgcaagaagagctgctgctcctgctgccccgtgggctgtgccaagtgtgcccacggctgtgtctgcaaagggacgttggagaactgcagctgctgtgcctga

SEQ ID NO: 18 - protein, 61 aa, Homo sapiens
MDPNCSCTTGVSCACTGSCKCKECKCTSCKKSCCSCCPVGCAKCAHGCVCKGTLENCSCCA

SEQ ID NO: 19 - DNA, 955 bp, Homo sapiens
ccaagagctaaaagagagtaagggggaaacaacagcagctccaaccagggcagccttcctgagaagatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacaagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagccaagtccagatttacactgggagaggtgccagcaactgaataaatacctcttagctgagtggaaaaaaaaaaaaaaaaaaaaaaaaaaaaaaaa

SEQ ID NO: 20 - DNA, 744 bp, Homo sapiens
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacaagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaa

SEQ ID NO: 21 - protein, 247 aa, Homo sapiens
MQPILLLLAFLLLPRADAGEIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 22 - DNA, 1702 bp, Homo sapiens
ggagtcgaccgctcgggcagcgcaccgccacgagagcccgggacgcgggaaagaccgaaaggaagaggaagaggcaccggtggccatggggctggaggcggcgcgcgagctggagtgcgcggcgctgggcacgctgctgcgggatccgcgggaggcggaacgcacgctgctgctggactgccgccccttcctggccttctgccggcgccacgtgcgcgccgcgcggccagtgccttggaacgcgctgctgcggcgccgcgcgcgcggccctcctgccgccgttctcgcctgcctgctgcccgaccgcgcgctgcggacgcgcctggtccgcggggagctggcgcgggccgtggtgctggacgagggcagtgcctcggtggcggagctccggcccgacagcccggctcatgtgctgctggccgcgctgctgcacgagacccgcgcggggcccactgccgtgtacttcctgcgaggaggcttcgacggcttccagggctgctgtcccgatctgtgctctgaggcccccgcccctgcgctgccgccaacaggggacaaaaccagccgctccgactccagggctcctgtctacgaccagggtggccctgtggagatcttgccctacctgttcctgggcagctgcagtcactcgtcagacctgcaggggctgcaggcctgtggcatcacagccgtcctcaacgtgtccgccagctgccccaaccactttgagggccttttccgctacaagagtatccctgtggaggacaaccagatggtggagatcagtgcctggttccaggaggccataggcttcattgactgggtgaagaacagcggaggccgggtgctggtgcactgccaggcgggtatctcgcgctctgccaccatctgtctggcatacctcatgcagagtcgccgtgtgcggctggacgaggcctttgacttcgttaagcagcgccggggggtcatctcccccaacttcagtttcatggggcagctgctgcagtttgagacccaggtgctgtgtcactgaggtggtgcccctctgcctgcctgccccactgtgctggcaggagctgactgtggactggtgggctcccctctgggccagcacagtcccctcacctccggcagggctgctacctcctcagagtttcagaagcccccacatgggggctctaggaatgccggcatgctggtctttccgacctggtgctcttctgctgggggactgaggctggccctcattcggggtcgggaaccaagggtgtgtctgctctttccctccccatcctctggcagaaatcagctagacgctataccgtggactctccctggtccaccaccatgttgaagcccttggcagcctgagagctccaaggaacaagctgtgacaaccaggagccctgtctgtgggttcgtctgcccagggcctggagcccaagccctgtgttcctggggaagctggggacttgggaagtgatgggtgtgtcatgttgcgtgtgtctgtctgtgagcctttcacacctgtgctggcgctggaaaattatttgtgctcagctgacatttaacacttcctcccccgcttcctcctagccctgtgggcaggggttggaaacttagcactttatatttatacagaacattcaggatttgtcaataaaatattgttatatttaaaaaaaaaaaaaaaaaaaaaaaaaaa

SEQ ID NO: 23 - DNA, 945 bp, Homo sapiens
atggggctggaggcggcgcgcgagctggagtgcgcggcgctgggcacgctgctgcgggatccgcgggaggcggaacgcacgctgctgctggactgccgccccttcctggccttctgccggcgccacgtgcgcgccgcgcggccagtgccttggaacgcgctgctgcggcgccgcgcgcgcggccctcctgccgccgttctcgcctgcctgctgcccgaccgcgcgctgcggacgcgcctggtccgcggggagctggcgcgggccgtggtgctggacgagggcagtgcctcggtggcggagctccggcccgacagcccggctcatgtgctgctggccgcgctgctgcacgagacccgcgcggggcccactgccgtgtacttcctgcgaggaggcttcgacggcttccagggctgctgtcccgatctgtgctctgaggcccccgcccctgcgctgccgccaacaggggacaaaaccagccgctccgactccagggctcctgtctacgaccagggtggccctgtggagatcttgccctacctgttcctgggcagctgcagtcactcgtcagacctgcaggggctgcaggcctgtggcatcacagccgtcctcaacgtgtccgccagctgccccaaccactttgagggccttttccgctacaagagtatccctgtggaggacaaccagatggtggagatcagtgcctggttccaggaggccataggcttcattgactgggtgaagaacagcggaggccgggtgctggtgcactgccaggcgggtatctcgcgctctgccaccatctgtctggcatacctcatgcagagtcgccgtgtgcggctggacgaggcctttgacttcgttaagcagcgccggggggtcatctcccccaacttcagtttcatggggcagctgctgcagtttgagacccaggtgctgtgtcactga

SEQ ID NO: 24 - protein, 314 aa, Homo sapiens
MGLEAARELECAALGTLLRDPREAERTLLLDCRPFLAFCRRHVRAARPVPWNALLRRRARGPPAAVLACLLPDRALRTRLVRGELARAVVLDEGSASVAELRPDSPAHVLLAALLHETRAGPTAVYFLRGGFDGFQGCCPDLCSEAPAPALPPTGDKTSRSDSRAPVYDQGGPVEILPYLFLGSCSHSSDLQGLQACGITAVLNVSASCPNHFEGLFRYKSIPVEDNQMVEISAWFQEAIGFIDWVKNSGGRVLVHCQAGISRSATICLAYLMQSRRVRLDEAFDFVKQRRGVISPNFSFMGQLLQFETQVLCH

SEQ ID NO: 25 - DNA, 1403 bp, Homo sapiens
gcctgtctgcattctactatataaagcagcagagacgttgactagcgcatatttgctaagagcaccatgcgcgcagcagccatctccactccaaagttagacaaaatgccaggaatgttcttctctgctaacccaaaggaattgaaaggaaccactcattcacttctagacgacaaaatgcaaaaaaggaggccaaagacttttggaatggatatgaaagcatacctgagatctatgatcccacatctggaatctggaatgaaatcttccaagtccaaggatgtactttctgctgctgaagtaatgcaatggtctcaatctctggaaaaacttcttgccaaccaaactggtcaaaatgtctttggaagtttcctaaagtctgaattcagtgaggagaatattgagttctggctggcttgtgaagactataagaaaacagagtctgatcttttgccctgtaaagcagaagagatatataaagcatttgtgcattcagatgctgctaaacaaatcaatattgacttccgcactcgagaatctacagccaagaagattaaagcaccaacccccacgtgttttgatgaagcacaaaaagtcatatatactcttatggaaaaggactcttatcccaggttcctcaaatcagatatttacttaaatcttctaaatgacctgcaggctaatagcctaaagtgactggtccctggctgaagggaattaacagatagtatcaagcgcagaaggaatgtgccagtatggctccctgggtgaacagcttggccttttttgggtgtcttgacaggccaagaagaacaaatgactcagaatggattaacatgaaagttatccaggcgcagagttgaagaagcataagcaagacaaaaacagagagaccgcagaaggaggaagatactgtggtactgtcataaaaaacagtggagctctgtattagaaagcccctcagaactgggaaggccaggtaactctagttacacagaaactgtgactaaagtctatgaaactgattacaacagactgtaagaatcaaagtcaactgacatctatgctacatattattatatagtttgtactgagctattgaagtcccattaacttaaagtatatgttttcaaattgccattgctactattgcttgtcggtgttattttattttattgtttttgactttggaagagatgaactgtgtatttaacttaagctattgctcttaaaaccagggagtcagaatatatttgtaagttaaatcattggtgctaataataaatgtggattttgtattaaaatatatagaagcaatttctgtttacatgtccttgctacttttaaaaacttgcatttattcctcagattttaaaaataaataaataattcatttaagattc

SEQ ID NO: 26 - DNA, 591 bp, Homo sapiens
atgccaggaatgttcttctctgctaacccaaaggaattgaaaggaaccactcattcacttctagacgacaaaatgcaaaaaaggaggccaaagacttttggaatggatatgaaagcatacctgagatctatgatcccacatctggaatctggaatgaaatcttccaagtccaaggatgtactttctgctgctgaagtaatgcaatggtctcaatctctggaaaaacttcttgccaaccaaactggtcaaaatgtctttggaagtttcctaaagtctgaattcagtgaggagaatattgagttctggctggcttgtgaagactataagaaaacagagtctgatcttttgccctgtaaagcagaagagatatataaagcatttgtgcattcagatgctgctaaacaaatcaatattgacttccgcactcgagaatctacagccaagaagattaaagcaccaacccccacgtgttttgatgaagcacaaaaagtcatatatactcttatggaaaaggactcttatcccaggttcctcaaatcagatatttacttaaatcttctaaatgacctgcaggctaatagcctaaagtga

SEQ ID NO: 27 - protein, 196 aa, Homo sapiens
MPGMFFSANPKELKGTTHSLLDDKMQKRRPKTFGMDMKAYLRSMIPHLESGMKSSKSKDVLSAAEVMQWSQSLEKLLANQTGQNVFGSFLKSEFSEENIEFWLACEDYKKTESDLLPCKAEEIYKAFVHSDAAKQINIDFRTRESTAKKIKAPTPTCFDEAQKVIYTLMEKDSYPRFLKSDIYLNLLNDLQANSLK

SEQ ID NO: 28 - DNA, 3338 bp, Homo sapiens
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 29 - DNA, 996 bp, Homo sapiens
atgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttga

SEQ ID NO: 30 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 31 - DNA, 980 bp, Homo sapiens
ggggcagacgtgggacgggaaggacggctgccgggactggcgcgcggggacactgggccgacgcgtggagtagcggggagagcgggaagcctgagggggcggggccggcgcgaggccgtgggtgcggcacgaggatgccggcggcgggacagcgcccgtaggcagccccacgggcagggcgcgcgggcggggcggggcgggccgggccagaggagctcccggcatgtcgcaagggctcccggccgccggcagcgtcctgcagaggagcgtcgcggcgcccgggaaccagccgcagccgcagccgcagcagcagagccctgaggatgatgacaggaaggtccgaaggagagaaaaaaaccgagttgctgctcagagaagtcggaagaagcagacccagaaggctgacaagctccatgaggaatatgagagcctggagcaagaaaacaccatgctgcggagagagatcgggaagctgacagaggagctgaagcacctgacagaggcactgaaggagcacgagaagatgtgcccgctgctgctctgccctatgaactttgtgccagtgcctccccggccggaccctgtggccggctgcttgccccgatgaagccggggacactcctctgcccagcaaggagccttggtcattttcatacctgggaggaaggcttttccttcacaattgtatacagggggcacctgtggccaggcctcctcctgggagctccaggaccagccagctgtgttccctgcagactgggctcagcccgacatccaacaggcgccaaactcacagagcccttgtgcagatccagcatggaggccaccctcaggagtgacttctcatccaccctggcagctagtaggttctgctgttatgcagagccatttcctctagaatttggataataaagatgcttattgtctctcccttctccagttctgggaatttacaggcacaatacacttccttttcctgg

SEQ ID NO: 32 - DNA, 384 bp, Homo sapiens
atgtcgcaagggctcccggccgccggcagcgtcctgcagaggagcgtcgcggcgcccgggaaccagccgcagccgcagccgcagcagcagagccctgaggatgatgacaggaaggtccgaaggagagaaaaaaaccgagttgctgctcagagaagtcggaagaagcagacccagaaggctgacaagctccatgaggaatatgagagcctggagcaagaaaacaccatgctgcggagagagatcgggaagctgacagaggagctgaagcacctgacagaggcactgaaggagcacgagaagatgtgcccgctgctgctctgccctatgaactttgtgccagtgcctccccggccggaccctgtggccggctgcttgccccgatga

SEQ ID NO: 33 - protein, 127 aa, Homo sapiens
MSQGLPAAGSVLQRSVAAPGNQPQPQPQQQSPEDDDRKVRRREKNRVAAQRSRKKQTQKADKLHEEYESLEQENTMLRREIGKLTEELKHLTEALKEHEKMCPLLLCPMNFVPVPPRPDPVAGCLPR

SEQ ID NO: 34 - DNA, 760 bp, Homo sapiens
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaa

SEQ ID NO: 35 - DNA, 300 bp, Homo sapiens
atgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttga

SEQ ID NO: 36 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 37 - DNA, 776 bp, Homo sapiens
aaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctagagctgagtgcctgaggggtccagaagcttcgaggcccagcgacctcggtgggccagtggggaggagcaggagcctgagccttgggaaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagcacgctcttttaaaaggt

SEQ ID NO: 38 - DNA, 279 bp, Homo sapiens
atgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctagagctgagtgcctga

SEQ ID NO: 39 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 40 - DNA, 1745 bp, Homo sapiens
gaggaggaaacaacttcccggttgctttcagacgcttcagagatcctctggaggcctgggggagcttttgagtactttatttcagttggtccctgagctcggtgagtggggcgggtagagccaccaggggaatcaacagtggtttctcgtgcccctcagggtcaggagcagtctgatcaaaaggagggcatccactgtccggggccattcccacagctcccggatgctgggtctggaggctgcgcccttcccctgcaggagctcagcccagtgggcagtctgaagatggccaattacacgctggcaccagaggatgaatatgatgtcctcatagaaggtgaactggagagcgatgaggcagagcaatgtgacaagtatgacgcccaggcactctcagcccagctggtgccatcactctgctctgctgtgtttgtgatcggtgtcctggacaatctcctggttgtgcttatcctggtaaaatataaaggactcaaacgcgtggaaaatatctatcttctaaacttggcagtttctaacttgtgtttcttgcttaccctgcccttctgggctcatgctgggggcgatcccatgtgtaaaattctcattggactgtacttcgtgggcctgtacagtgagacatttttcaattgccttctgactgtgcaaaggtacctagtgtttttgcacaagggaaactttttctcagccaggaggagggtgccctgtggcatcattacaagtgtcctggcatgggtaacagccattctggccactttgcctgaattcgtggtttataaacctcagatggaagaccagaaatacaagtgtgcatttagcagaactcccttcctgccagctgatgagacattctggaagcattttctgactttaaaaatgaacatttcggttcttgtcctccccctatttatttttacatttctctatgtgcaaatgagaaaaacactaaggttcagggagcagaggtatagccttttcaagcttgtttttgccataatggtagtcttccttctgatgtgggcgccctacaatattgcatttttcctgtccactttcaaagaacacttctccctgagtgactgcaagagcagctacaatctggacaaaagtgttcacatcactaaactcatcgccaccacccactgctgcatcaaccctctcctgtatgcgtttcttgatgggacatttagcaaatacctctgccgctgtttccatctgcgtagtaacaccccacttcaacccagggggcagtctgcacaaggcacatcgagggaagaacctgaccattccaccgaagtgtaaactagcatccaccaaatgcaagaagaataaacatggattttcatctttctgcattatttcatgtaaattttctacacatttgtatacaaaatcggatacaggaagaaaagggagaggtgagctaacatttgctaagcactgaatttgtctcaggcaccgtgcaaggctctttacaaacgtgagctccttcgcctcctaccacttgtccatagtgtggataggactagtctcatttctctgagaagaaaactaaggcgcggaaatttgtctaagatcacataactaggaagtggcagaactgattctccagccctggtagcatttgctcagagcctacgcttggtccagaacatcaaactccaaaccctggggacaaacgacatgaaataaatgtattttaaaacatctaaaaaaaaaaaaaaaaa

SEQ ID NO: 41 - DNA, 1035 bp, Homo sapiens
atggccaattacacgctggcaccagaggatgaatatgatgtcctcatagaaggtgaactggagagcgatgaggcagagcaatgtgacaagtatgacgcccaggcactctcagcccagctggtgccatcactctgctctgctgtgtttgtgatcggtgtcctggacaatctcctggttgtgcttatcctggtaaaatataaaggactcaaacgcgtggaaaatatctatcttctaaacttggcagtttctaacttgtgtttcttgcttaccctgcccttctgggctcatgctgggggcgatcccatgtgtaaaattctcattggactgtacttcgtgggcctgtacagtgagacatttttcaattgccttctgactgtgcaaaggtacctagtgtttttgcacaagggaaactttttctcagccaggaggagggtgccctgtggcatcattacaagtgtcctggcatgggtaacagccattctggccactttgcctgaattcgtggtttataaacctcagatggaagaccagaaatacaagtgtgcatttagcagaactcccttcctgccagctgatgagacattctggaagcattttctgactttaaaaatgaacatttcggttcttgtcctccccctatttatttttacatttctctatgtgcaaatgagaaaaacactaaggttcagggagcagaggtatagccttttcaagcttgtttttgccataatggtagtcttccttctgatgtgggcgccctacaatattgcatttttcctgtccactttcaaagaacacttctccctgagtgactgcaagagcagctacaatctggacaaaagtgttcacatcactaaactcatcgccaccacccactgctgcatcaaccctctcctgtatgcgtttcttgatgggacatttagcaaatacctctgccgctgtttccatctgcgtagtaacaccccacttcaacccagggggcagtctgcacaaggcacatcgagggaagaacctgaccattccaccgaagtgtaa

SEQ ID NO: 42 - protein, 344 aa, Homo sapiens
MANYTLAPEDEYDVLIEGELESDEAEQCDKYDAQALSAQLVPSLCSAVFVIGVLDNLLVVLILVKYKGLKRVENIYLLNLAVSNLCFLLTLPFWAHAGGDPMCKILIGLYFVGLYSETFFNCLLTVQRYLVFLHKGNFFSARRRVPCGIITSVLAWVTAILATLPEFVVYKPQMEDQKYKCAFSRTPFLPADETFWKHFLTLKMNISVLVLPLFIFTFLYVQMRKTLRFREQRYSLFKLVFAIMVVFLLMWAPYNIAFFLSTFKEHFSLSDCKSSYNLDKSVHITKLIATTHCCINPLLYAFLDGTFSKYLCRCFHLRSNTPLQPRGQSAQGTSREEPDHSTEV

SEQ ID NO: 43 - DNA, 2687 bp, Homo sapiens
gctggcggccggcagggggcggagttcgagcctggatttttttcctcggggcctcccccgggaggccgtcccggcgtgggggaggggaggacggggcgggaggacgcggttcggtcggctgcagcgctacttttggtccggggtcggcagggaggccgcggctaccgcatcacagctgacgtgaggactacgtggggccgctgccggctccgggttgctgggcggcggcgccgctgctgagcggcggtcgggctcgccgtctccacctcctcgcgtccgtaatcagtgacgaggtccgctacgtaaatccctttgcggcgggataaataaagaaaacaggaaaggaggaaagcattgattacaaatatcttaacaatgagcaaatgtgcaaggaaaaaatatattaagacaaatccaagacaaatgaccatggaaacagttgaatcccagcatgatggaagtataacagcttctttgacagagagcaagtctgctcatgtgcagactcagactggccaaaattcaatccctgctttagctcaggtagcagcaattgcagagacagatgaatctgcagaatcagaaggtgtaattgattctcataaacgtagagaaatcctttcacgaagaccctcttataggaaaatactgaatgaactgtcctctgatgtgcctggtgttcccaagattgaagaagagagatcagaggaagaaggaacaccacctagtattgctaccatggcagtaccaactagcatatatcagactagcacggggcaatacaatgaggaaactgaacttgccccaagtcacatggctgctgccactggtgacatgccaacttaccagatccgagctcctactgctgctttgccacagggagtggtgatggctgcatcgcccggaagtttgcacagtccccagcagctggcagaagaagcaacacgcaaacgagagctgaggctaatgaaaaacagggaagctgcccgggagtgtcgcaggaagaagaaagaatatgtcaaatgtcttgaaaatcgtgtggctgtgcttgaaaaccaaaacaagactctcattgaggaactcaaggccctcaaagatctttattgccataaagtagagtaactgtctttgacttggaccttgtttactctaatcaaggcaggagatgcagcagtcctacttattgccatgtggacttgtgggaaggacacgtgtgacccttaagaatccagtttggattagtgtttgaaattgaattgggaatgttgttccaggatgtggaatgcagccgtgatcacacttaccgagcttactttgatctgtttgtcaatagcatgcaaaaaatgctttgtttgccctttgcttctgctttttttcagggaagctgccaaagaatgtcgacgtcgaaagaaagaatatgtaaaatgtctggagagccgagttgcagtgctggaagtccagaacaagaagcttatagaggaacttgaaaccttgaaagacatttgttctcccaaaacagattactagaaatatttaactatgaactgaaggcagcatgtatagttgcttttgaaggaatacaatatatagctggcaagaatggtggcttcttttctttgtatcattcatcttcttctttaatcacttaacattcctaaaatgcttcactgtacgtagttaagtcgtagctataacttcaaattttttaaaagagacaaactgtaaaaaatgtgtgtattcttaaaatgcaatatttgtaaggcttgttccaatgccacatacttgcagctcccattctatgtgtcatcaatagtgtcctatgcaataaaattatttgcaggtctttaaatcattttaggaaaggatgatcaaaaataatgcatccagcagtacaataaaagtaaaccacaaaaaaatacctcaggaaagaatagaaagaaagtctatctaatgacatgcctatatgagaagaatagcctagatatgaatatatggcatttgcagatttttatattagttgctttgttaaaaaaaaaaagattgtattgctgtccttgaatgccatagtcaaagagagtttttaatagaaccatgttggttgcactttgtagtgtttggtgctcatttaaatatctgaacatttactacagtttttaactctactgtgtaatataaaagatcttgcagaagttcttagtgttggtttaatattacctaatgaaagtgatgacatattttttatatctggaatgagcctgttgggatcgcattgcataccttcggggtacaagtcagtttctacactgggtgctgatccttgcacgcgccctttctaccatctcacggtggggatggccgcagggctgtgcacccagaagaagatggctgttagcgtttcggccttcataatggcctgagactttctttctgtaggtggtctggagctgtccggctggtggccccctattttgccatttagcgaacaaccacaggaattttaaaaacaaaaacatcccaagattttttcatttcaaaatgcttcaaagtccacattagatcagatactccgctgtcggcacattcagctgaggttcattacaatcgagactgcaatgtgatctatgtttcatcttgtttttataataaaaagcttcagggaacaagcccaaagccctcaccacaaaaaa

SEQ ID NO: 44 - DNA, 747 bp, Homo sapiens
atgagcaaatgtgcaaggaaaaaatatattaagacaaatccaagacaaatgaccatggaaacagttgaatcccagcatgatggaagtataacagcttctttgacagagagcaagtctgctcatgtgcagactcagactggccaaaattcaatccctgctttagctcaggtagcagcaattgcagagacagatgaatctgcagaatcagaaggtgtaattgattctcataaacgtagagaaatcctttcacgaagaccctcttataggaaaatactgaatgaactgtcctctgatgtgcctggtgttcccaagattgaagaagagagatcagaggaagaaggaacaccacctagtattgctaccatggcagtaccaactagcatatatcagactagcacggggcaatacaatgaggaaactgaacttgccccaagtcacatggctgctgccactggtgacatgccaacttaccagatccgagctcctactgctgctttgccacagggagtggtgatggctgcatcgcccggaagtttgcacagtccccagcagctggcagaagaagcaacacgcaaacgagagctgaggctaatgaaaaacagggaagctgcccgggagtgtcgcaggaagaagaaagaatatgtcaaatgtcttgaaaatcgtgtggctgtgcttgaaaaccaaaacaagactctcattgaggaactcaaggccctcaaagatctttattgccataaagtagagtaa

SEQ ID NO: 45 - protein, 248 aa, Homo sapiens
MSKCARKKYIKTNPRQMTMETVESQHDGSITASLTESKSAHVQTQTGQNSIPALAQVAAIAETDESAESEGVIDSHKRREILSRRPSYRKILNELSSDVPGVPKIEEERSEEEGTPPSIATMAVPTSIYQTSTGQYNEETELAPSHMAAATGDMPTYQIRAPTAALPQGVVMAASPGSLHSPQQLAEEATRKRELRLMKNREAARECRRKKKEYVKCLENRVAVLENQNKTLIEELKALKDLYCHKVE

SEQ ID NO: 46 - DNA, 4004 bp, Homo sapiens
ccttttgcagcaagttctttcctgcactaatcacaattcttggaagaggagaactggacgttgtgaacagagttagctggtaaatgtcctcttaaaagatccaaaaaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagaatcaatcataaagtgcacacctttattcagaactttagtattaaatcagttctcaatttcattttttatgtattgttttactcctttttattcatacgtaaaattttggattaatttgtgaaaatgtaattataagctgagaccggtggctctcttcttaaaagcaccatattaaatcctggaaaactaaaaaaaaaaaa

SEQ ID NO: 47 - DNA, 3696 bp, Homo sapiens
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 48 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 49 - DNA, 1216 bp, Homo sapiens
ggcagctgcacggctcctggccccggagcatgcgcgagagccgccccggagcgccccggagccccccgccgtcccgcccgcggcgtcccgcgccccgccgccagcgcacccccggacgctatggcccacccctccggctggccccttctgtaggatggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttgaccggcagcgcccgccgtgcacgcagcattaactgggatgccgtgttattttgttattacttgcctggaaccatgtgggtaccctccccggcctgggttggagggagcggatgggtgtaggggcgaggcgcctcccgccctcggctggagacgaggccgcagaccccttctcacctcttgagggggtcctccccctcctggtgctccctctgggtccccctggttgttgtagcagcttaactgtatctggagccaggacctgaactcgcacctcctacctcttcatgtttacatatacccagtatctttgcacaaaccaggggttgggggagggtctctggctttatttttctgctgtgcagaatcctattttatattttttaaagtcagtttaggtaataaactttattatgaaagtttttttttt

SEQ ID NO: 50 - DNA, 636 bp, Homo sapiens
atggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttga

SEQ ID NO: 51 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 52 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 53 - DNA, 501 bp, Homo sapiens
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 54 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 55 - DNA, 1940 bp, Homo sapiens
acccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgacagccacctaactgcccctgcagaaggagcaatggccttggctcctgagaggtaagagcccggcccaccctctccagatgccagtccccgagcgccctgcagccggccctgactctccgcggccgggcacccgcagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgagataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacgagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggccccccaggctccgccagcccctccaccccagcctgcctgctgcctcggaactgtgacacccccatcttcgacttcagagtcttcttccgagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggcc

SEQ ID NO: 56 - DNA, 1551 bp, Homo sapiens
atgccagtccccgagcgccctgcagccggccctgactctccgcggccgggcacccgcagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgagataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacgagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggccccccaggctccgccagcccctccaccccagcctgcctgctgcctcggaactgtgacacccccatcttcgacttcagagtcttcttccgagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctag

SEQ ID NO: 57 - protein, 516 aa, Homo sapiens
MPVPERPAAGPDSPRPGTRRAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDEGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFRELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 58 - DNA, 2892 bp, Homo sapiens
caaagatggctgccacattggcgctgtcattttggtactgagcagagcgacgggcttaattcgacccaatccaggccagagtctttctctcaggggcttcctcgtgctcagctaatcctccgatcaatccttgggaatccctgggacctcttcggtatccctactctcagccagggatcatgtcttgggccgctcgcccgcccttcctccctcagcggcatgccgcagggcagtgtgggccggtgggggtgcgaaaagaaatgcattgtggggtcgcgtcccggtggcggcggcgacggccctggctggatcccgcagcggcggcggcggcggcggtggcaggcggagaacaacaaaccccggagccggagccaggggaggctggacgggacgggatgggcgacagcgggcgggactcccgaagcccagacagctcctccccaaatccccttccccagggagtccctcccccttctcctcctgggccacccctacccccttcaacagctccatcccttggaggctctggggccccacccccacccccgatgccaccacccccactgggctctccctttccagtcatcagttcttccatggggtcccctggtctgccccctccagctcccccaggattctccgggcctgtcagcagcccccagattaactcaacagtgtcactccctgggggtgggtctggcccccctgaagatgtgaagccaccagtcttaggggtccggggcctgcactgtccaccccctccaggtggccctggggctggcaaacggctatgtgcaatctgcggggacagaagctcaggcaaacactacggggtttacagctgtgagggttgcaagggcttcttcaaacgcaccatccgcaaagaccttacatactcttgccgggacaacaaagactgcacagtggacaagcgccagcggaaccgctgtcagtactgccgctatcagaagtgcctggccactggcatgaagagggaggcggtacaggaggagcgtcagcggggaaaggacaaggatggggatggggagggggctgggggagcccccgaggagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttctcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagatgccaagggcctctccaaccctagtgaggtggaggtcctgcgggagaaagtgtatgcatcactggagacctactgcaaacagaagtaccctgagcagcagggacggtttgccaagctgctgctacgtcttcctgccctccggtccattggccttaagtgtctagagcatctgtttttcttcaagctcattggtgacacccccatcgacaccttcctcatggagatgcttgaggctccccatcaactggcctgagctcagacccagacgtggtgcttctcacactggaggagcacacatccaagagggactccaagccctggggcagggtggggggccatgttcccagaaccttgatggggtgagaagtacagggcagaaccaagaacataaaccctccaagggatctgcttgatatcccaagttggaagggaccccagatacctgtgaggactggttgtctctcttcggtggccttgagtctctgaatttgtcgggttctcccatgatttggggtgatttctcaccctctgtccttcccccagcacaaagcactggccttgcctccaggaccttgcttccttctcatcttgcctcattttgcttcccatctgaagagtggaaatggggaactcccccagaggtggatactggggggcaggcctcccaagctgatggacatgagagtagggccctgacaggccttcctcctctcaaacctggcagatgggggcctctctggaagagggaggggccctgtcactgtccagagtctctttttacacttcacctccttctgcagtcagactgaaatataaaaaaggtggtggtggtggtgaaggggctggtggagatgtaggaaccgatctgctatttttaatttcctgtgaggatagagacttgcagttagactcaaagaagtactgtactttcccaggttgactaagaaatgccagtggtggaggtgggtgtttgggaaaggcagggccctgaaatggcctgtccctagggctctccaagcactagccttcccagcttcccgccgccccccctatctcttcctgtctaacttggggaaggggcctgggctgtgaggacagggcccccacaggggatggtttcacgagtgtagtcccggaggccttccctttacagctctcctccagccctgggcacatagcataggctggggacacaggatcctggcctgagaattgaggggaggtggccagcccgcagaggtggggtgctggggctgcatgatttttgccctgcgtcccttctctttggggctcctttcccctctcatacataaaatcgctttcaaattaaaatcgctgttttctggaaaaaaaaaaaaaaaaaa

SEQ ID NO: 59 - DNA, 1602 bp, Homo sapiens
atgtcttgggccgctcgcccgcccttcctccctcagcggcatgccgcagggcagtgtgggccggtgggggtgcgaaaagaaatgcattgtggggtcgcgtcccggtggcggcggcgacggccctggctggatcccgcagcggcggcggcggcggcggtggcaggcggagaacaacaaaccccggagccggagccaggggaggctggacgggacgggatgggcgacagcgggcgggactcccgaagcccagacagctcctccccaaatccccttccccagggagtccctcccccttctcctcctgggccacccctacccccttcaacagctccatcccttggaggctctggggccccacccccacccccgatgccaccacccccactgggctctccctttccagtcatcagttcttccatggggtcccctggtctgccccctccagctcccccaggattctccgggcctgtcagcagcccccagattaactcaacagtgtcactccctgggggtgggtctggcccccctgaagatgtgaagccaccagtcttaggggtccggggcctgcactgtccaccccctccaggtggccctggggctggcaaacggctatgtgcaatctgcggggacagaagctcaggcaaacactacggggtttacagctgtgagggttgcaagggcttcttcaaacgcaccatccgcaaagaccttacatactcttgccgggacaacaaagactgcacagtggacaagcgccagcggaaccgctgtcagtactgccgctatcagaagtgcctggccactggcatgaagagggaggcggtacaggaggagcgtcagcggggaaaggacaaggatggggatggggagggggctgggggagcccccgaggagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttctcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagatgccaagggcctctccaaccctagtgaggtggaggtcctgcgggagaaagtgtatgcatcactggagacctactgcaaacagaagtaccctgagcagcagggacggtttgccaagctgctgctacgtcttcctgccctccggtccattggccttaagtgtctagagcatctgtttttcttcaagctcattggtgacacccccatcgacaccttcctcatggagatgcttgaggctccccatcaactggcctga

SEQ ID NO: 60 - protein, 533 aa, Homo sapiens
MSWAARPPFLPQRHAAGQCGPVGVRKEMHCGVASRWRRRRPWLDPAAAAAAAVAGGEQQTPEPEPGEAGRDGMGDSGRDSRSPDSSSPNPLPQGVPPPSPPGPPLPPSTAPSLGGSGAPPPPPMPPPPLGSPFPVISSSMGSPGLPPPAPPGFSGPVSSPQINSTVSLPGGGSGPPEDVKPPVLGVRGLHCPPPPGGPGAGKRLCAICGDRSSGKHYGVYSCEGCKGFFKRTIRKDLTYSCRDNKDCTVDKRQRNRCQYCRYQKCLATGMKREAVQEERQRGKDKDGDGEGAGGAPEEMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPDAKGLSNPSEVEVLREKVYASLETYCKQKYPEQQGRFAKLLLRLPALRSIGLKCLEHLFFFKLIGDTPIDTFLMEMLEAPHQLA

SEQ ID NO: 61 - DNA, 3384 bp, Homo sapiens
tgtttatactccggagggtgtccccgtgcgtcatcggtggagtggaccaaaactggtgatctgtttgccctgtgtgaccttgcccagaaccctgctgactgagagaacacatctgctggaagtcctctgggattcaaggtacagggaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctgtcctgtggaggaaggaccaacaccatcaccgcgtggagcagctgaccctgaatggcctcctgcaggctcttcagagcccctgcatcattgaaggggaatctggcaaaggcaagtccactctgctgcagcgaattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccactgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagtttattgacgtctcatgagccagaggaggtgaccaaggggaatggttacttgcagaaaatggtttccatttcggacattacatccacttatagcagcctgctccggtacacctgtgggtcatctgtggaagccaccagggctgttatgaagcacctcgcagcagtgtatcaacacggctgccttctcggactttccatcgccaagaggcctctctggagacaggaatctttgcaaagtgtgaaaaacaccactgagcaagaaattctgaaagccataaacatcaattcctttgtagagtgtggcatccatttatatcaagagagtacatccaaatcagccctgagccaagaatttgaagctttctttcaaggtaaaagcttatatatcaactcagggaacatccccgattacttatttgacttctttgaacatttgcccaattgtgcaagtgccctggacttcattaaactggacttttatgggggagctatggcttcatgggaaaaggctgcagaagacacaggtggaatccacatggaagaggccccagaaacctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagagatgtgctggtgtggctggaagcctcagtttggtcctcagcacctgtaagaacatttattctctcatggtggaagccagtcccctcaccatagaagatgagaggcacatcacatctgtaacaaacctgaaaaccttgagtattcatgacctacagaatcaacggctgccgggtggtctgactgacagcttgggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaagcaattagtgttttttgactttagtactaaagaatttctacctgatccagcattagtcagaaaacttagccaagtgttatccaagttaacttttctgcaagaagctaggcttgttgggtggcaatttgatgatgatgatctcagtgttattacaggtgcttttaaactagtaactgcttaaataaagtgtactcgaagccagtaaaaaaaaaaaaaaaaaaaaaaa

SEQ ID NO: 62 - DNA, 3075 bp, Homo sapiens
atgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctgtcctgtggaggaaggaccaacaccatcaccgcgtggagcagctgaccctgaatggcctcctgcaggctcttcagagcccctgcatcattgaaggggaatctggcaaaggcaagtccactctgctgcagcgaattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccactgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagtttattgacgtctcatgagccagaggaggtgaccaaggggaatggttacttgcagaaaatggtttccatttcggacattacatccacttatagcagcctgctccggtacacctgtgggtcatctgtggaagccaccagggctgttatgaagcacctcgcagcagtgtatcaacacggctgccttctcggactttccatcgccaagaggcctctctggagacaggaatctttgcaaagtgtgaaaaacaccactgagcaagaaattctgaaagccataaacatcaattcctttgtagagtgtggcatccatttatatcaagagagtacatccaaatcagccctgagccaagaatttgaagctttctttcaaggtaaaagcttatatatcaactcagggaacatccccgattacttatttgacttctttgaacatttgcccaattgtgcaagtgccctggacttcattaaactggacttttatgggggagctatggcttcatgggaaaaggctgcagaagacacaggtggaatccacatggaagaggccccagaaacctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagagatgtgctggtgtggctggaagcctcagtttggtcctcagcacctgtaagaacatttattctctcatggtggaagccagtcccctcaccatagaagatgagaggcacatcacatctgtaacaaacctgaaaaccttgagtattcatgacctacagaatcaacggctgccgggtggtctgactgacagcttgggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaagcaattagtgttttttgactttagtactaaagaatttctacctgatccagcattagtcagaaaacttagccaagtgttatccaagttaacttttctgcaagaagctaggcttgttgggtggcaatttgatgatgatgatctcagtgttattacaggtgcttttaaactagtaactgcttaa

SEQ ID NO: 63 - protein, 1024 aa, Homo sapiens
MNFIKDNSRALIQRMGMTVIKQITDDLFVWNVLNREEVNIICCEKVEQDAARGIIHMILKKGSESCNLFLKSLKEWNYPLFQDLNGQSLFHQTSEGDLDDLAQDLKDLYHTPSFLNFYPLGEDIDIIFNLKSTFTEPVLWRKDQHHHRVEQLTLNGLLQALQSPCIIEGESGKGKSTLLQRIAMLWGSGKCKALTKFKFVFFLRLSRAQGGLFETLCDQLLDIPGTIRKQTFMAMLLKLRQRVLFLLDGYNEFKPQNCPEIEALIKENHRFKNMVIVTTTTECLRHIRQFGALTAEVGDMTEDSAQALIREVLIKELAEGLLLQIQKSRCLRNLMKTPLFVVITCAIQMGESEFHSHTQTTLFHTFYDLLIQKNKHKHKGVAASDFIRSLDHCGDLALEGVFSHKFDFELQDVSSVNEDVLLTTGLLCKYTAQRFKPKYKFFHKSFQEYTAGRRLSSLLTSHEPEEVTKGNGYLQKMVSISDITSTYSSLLRYTCGSSVEATRAVMKHLAAVYQHGCLLGLSIAKRPLWRQESLQSVKNTTEQEILKAININSFVECGIHLYQESTSKSALSQEFEAFFQGKSLYINSGNIPDYLFDFFEHLPNCASALDFIKLDFYGGAMASWEKAAEDTGGIHMEEAPETYIPSRAVSLFFNWKQEFRTLEVTLRDFSKLNKQDIRYLGKIFSSATSLRLQIKRCAGVAGSLSLVLSTCKNIYSLMVEASPLTIEDERHITSVTNLKTLSIHDLQNQRLPGGLTDSLGNLKNLTKLIMDNIKMNEEDAIKLAEGLKNLKKMCLFHLTHLSDIGEGMDYIVKSLSSEPCDLEEIQLVSCCLSANAVKILAQNLHNLVKLSILDLSENYLEKDGNEALHELIDRMNVLEQLTALMLPWGCDVQGSLSSLLKHLEEVPQLVKLGLKNWRLTDTEIRILGAFFGKNPLKNFQQLNLAGNRVSSDGWLAFMGVFENLKQLVFFDFSTKEFLPDPALVRKLSQVLSKLTFLQEARLVGWQFDDDDLSVITGAFKLVTA

SEQ ID NO: 64 - DNA, 4145 bp, Homo sapiens
gggtggcctggtgtgtgggcgcggcagggcgcaggcgcaggcgcagtgtgcgtccgcgtctgaggggagggatgtgggggaagcgacggcccccggtttgtttgggctgtgggcggtgcgcagcggagagcccgggaaaagcgggaaatggcggcgccgagcgcggggtcttggtccaccttccagcacaaggagctgatggccgctgacaggggacgcaggatattgggagtgtgtggcatgcatcctcatcatcaggaaactctaaaaaagaaccgagtggtgctagccaaacagctgttgttgagcgaattgttagaacatcttctggagaaggacatcatcaccttggaaatgagggagctcatccaggccaaagtgggcagtttcagccagaatgtggaactcctcaacttgctgcctaagaggggtccccaagcttttgatgccttctgtgaagcactgagggagaccaagcaaggccacctggaggatatgttgctcaccaccctttctgggcttcagcatgtactcccaccgttgagctgtgactacgacttgagtctcccttttccggtgtgtgagtcctgtcccctttacaagaagctccgcctgtcgacagatactgtggaacactccctagacaataaagatggtcctgtctgccttcaggtgaagccttgcactcctgaattttatcaaacacacttccagctggcatataggttgcagtctcggcctcgtggcctagcactggtgttgagcaatgtgcacttcactggagagaaagaactggaatttcgctctggaggggatgtggaccacagtactctagtcaccctcttcaagcttttgggctatgacgtccatgttctatgtgaccagactgcacaggaaatgcaagagaaactgcagaattttgcacagttacctgcacaccgagtcacggactcctgcatcgtggcactcctctcgcatggtgtggagggcgccatctatggtgtggatgggaaactgctccagctccaagaggtttttcagctctttgacaacgccaactgcccaagcctacagaacaaaccaaaaatgttcttcatccaggcctgccgtggagatgagactgatcgtggggttgaccaacaagatggaaagaaccacgcaggatcccctgggtgcgaggagagtgatgccggtaaagaaaagttgccgaagatgagactgcccacgcgctcagacatgatatgcggctatgcctgcctcaaagggactgccgccatgcggaacaccaaacgaggttcctggtacatcgaggctcttgctcaagtgttttctgagcgggcttgtgatatgcacgtggccgacatgctggttaaggtgaacgcacttatcaaggatcgggaaggttatgctcctggcacagaattccaccggtgcaaggagatgtctgaatactgcagcactctgtgccgccacctctacctgttcccaggacaccctcccacatgatgtcacctccccatcatccacgccaagtggaagccactggaccacaggaggtgtgatagagcctttgatcttcaggatgcacggtttctgttctgccccctcagggatgtgggaatctcccagacttgtttcctgtgcccatcatctctgcctttgagtgtgggactccaggccagctccttttctgtgaagccctttgcctgtagagccagccttggttggacctattgccaggaatgtttcagctgcagttgaagagcctgacaagtgaagttgtaaacacagtgtggttatggggagagggcatataaattccccatatttgtgttcagttccagcttttgtagatggcactttagtgattgcttttattacattagttaagatgtctgagagaccatctcctatcttttatttcattcatatcctccgccctttttgtcctagagtgagagtttggaaggtgtccaaatttaatgtagacattatcttttggctctgaagaagcaaacatgactagagacgcaccttgctgcagtgtccagaagcggcctgtgcgttcccttcagtactgcagcgccacccagtggaaggacactcttggctcgtttgggctcaaggcaccgcagcctgtcagccaacattgccttgcatttgtaccttattgatctttgcccatggaagtctcaaagatctttcgttggttgtttctctgagctttgttactgaaatgagcctcgtggggagcatcagagaaggccaggaagaatggtgtgtttccctagactctgtaaccacctctctgtctttttccttcctgagaaacgtccatctctctcccttactattcccactttcattcaatcaacctgcacttcatatctagatttctagaaaagcttcctagcttatctccctgcttcatatctctcccttctttaccttcatttcatcctgttggctgctgccaccaaatctgtctagaatcctgctttacaggatcatgtaaatgctcaaagatgtaatgtagttctttgttcctgctttctctttcagtattaaactctcctttgatattatgtggcttttatttcagtgccatacatgttattgttttcaacctagaaacctttatccctgcttatctgaaacttcccaacttccctgttctttaagactttttttttttttttttttttttttgagacagagtctcgctctgtcgcccaggctggagggcagtggcacgatctcagctcactgcaagctccaactcccgggttcacgccattctcctgcctcagccttccaagtagctgggactacaggtgcccgccaccgtgcccggctaatttttttgtatttttagtagagacagggtttcaccatgttagccgggatggtcttgatctcctgacctcatgatccacccacctcagcctcccaaagtgttgggattacaggcgtgagccactgcgcccgggcaagacctttttttaaaaaaaaaaaaaaaaaaacttccattctttcttcctccagtctgttctcacataacagagtagttttggtttttaattttttttggttgtttgctgttttttgttttttaaggtgagttctcactatgtttctcagactggtctcgaactcctggcctcaagccatcttcccgcctcagcctctcaaatagctgggcttacaggcatgagccaccacacctggccaggatttggttgtttaaatataaatctgatcacccccctgcttagaacccttctgctttctattacccctcatttaaaatgtaaactcttcaccttggtttatgagaactggttcttgccttccccttgaacctcattaaatggtgatttcttgctaagctccagcccgagtggtctcctctcagcttctaattttgtgctctttcctgcccttttcctgggccttctcagctctccacccccaccactcttgactcaggtggtgtccttcttcctcaagtcttgacaattcccgggcccttcagtccctgagcagtctacttctgtgtctgtcaccacatcttgtcttttcccctcattgcatttattgcagtttatatatatgctacttttacttgttcatttctgtctcccctaccaggctgtaaatgagggcagaaaccttgtttgttttattcaccatcatgtaccaagtgcttggcacatagtgggccttcattaaatgtttgttgaataaaagagggaagaaggcaagccaaccttagctacaatcctaccttttgataaaatgttccttttgacaatatacacggattattatttgtactttgtttttccatgtgttttgcttttatccactggcatttttagctccttgaagacatatcatgtgtgagataacttccttcacatctcccatggtccctagcaaaatgctaggcctgtagtagtcaaggtgctcaataaatatttgtttgggtggtttgtgagccttgctgccaagtcctgcctttgggtcgacatagtatggaagtatttgagagagagaacctttccactcccactgccaggattttgtattgccatcgggtgccaaataaatgctcatatttattaaaaaaaaaaaaaaaaaaa

SEQ ID NO: 65 - DNA, 1359 bp, Homo sapiens
atggcggcgccgagcgcggggtcttggtccaccttccagcacaaggagctgatggccgctgacaggggacgcaggatattgggagtgtgtggcatgcatcctcatcatcaggaaactctaaaaaagaaccgagtggtgctagccaaacagctgttgttgagcgaattgttagaacatcttctggagaaggacatcatcaccttggaaatgagggagctcatccaggccaaagtgggcagtttcagccagaatgtggaactcctcaacttgctgcctaagaggggtccccaagcttttgatgccttctgtgaagcactgagggagaccaagcaaggccacctggaggatatgttgctcaccaccctttctgggcttcagcatgtactcccaccgttgagctgtgactacgacttgagtctcccttttccggtgtgtgagtcctgtcccctttacaagaagctccgcctgtcgacagatactgtggaacactccctagacaataaagatggtcctgtctgccttcaggtgaagccttgcactcctgaattttatcaaacacacttccagctggcatataggttgcagtctcggcctcgtggcctagcactggtgttgagcaatgtgcacttcactggagagaaagaactggaatttcgctctggaggggatgtggaccacagtactctagtcaccctcttcaagcttttgggctatgacgtccatgttctatgtgaccagactgcacaggaaatgcaagagaaactgcagaattttgcacagttacctgcacaccgagtcacggactcctgcatcgtggcactcctctcgcatggtgtggagggcgccatctatggtgtggatgggaaactgctccagctccaagaggtttttcagctctttgacaacgccaactgcccaagcctacagaacaaaccaaaaatgttcttcatccaggcctgccgtggagatgagactgatcgtggggttgaccaacaagatggaaagaaccacgcaggatcccctgggtgcgaggagagtgatgccggtaaagaaaagttgccgaagatgagactgcccacgcgctcagacatgatatgcggctatgcctgcctcaaagggactgccgccatgcggaacaccaaacgaggttcctggtacatcgaggctcttgctcaagtgttttctgagcgggcttgtgatatgcacgtggccgacatgctggttaaggtgaacgcacttatcaaggatcgggaaggttatgctcctggcacagaattccaccggtgcaaggagatgtctgaatactgcagcactctgtgccgccacctctacctgttcccaggacaccctcccacatga

SEQ ID NO: 66 - protein, 452 aa, Homo sapiens
MAAPSAGSWSTFQHKELMAADRGRRILGVCGMHPHHQETLKKNRVVLAKQLLLSELLEHLLEKDIITLEMRELIQAKVGSFSQNVELLNLLPKRGPQAFDAFCEALRETKQGHLEDMLLTTLSGLQHVLPPLSCDYDLSLPFPVCESCPLYKKLRLSTDTVEHSLDNKDGPVCLQVKPCTPEFYQTHFQLAYRLQSRPRGLALVLSNVHFTGEKELEFRSGGDVDHSTLVTLFKLLGYDVHVLCDQTAQEMQEKLQNFAQLPAHRVTDSCIVALLSHGVEGAIYGVDGKLLQLQEVFQLFDNANCPSLQNKPKMFFIQACRGDETDRGVDQQDGKNHAGSPGCEESDAGKEKLPKMRLPTRSDMICGYACLKGTAAMRNTKRGSWYIEALAQVFSERACDMHVADMLVKVNALIKDREGYAPGTEFHRCKEMSEYCSTLCRHLYLFPGHPPT

SEQ ID NO: 67 - DNA, 4977 bp, Homo sapiens
cggggcccggggggcgggggcctgacggccgggccgggcggcggagctgcaagggacagaggcgcggcaggcgcgcggagccagcggagccagctgagcccgagcccagcccgcgcccgcgccgccatgcccctggccttctgcggcagcgagaaccactcggccgcctaccgggtggaccagggggtcctcaacaacggctgctttgtggacgcgctcaacgtggtgccgcacgtcttcctactcttcatcaccttccccatcctcttcattggatggggaagtcagagctccaaggtgcacatccaccacagcacatggcttcatttccccgggcacaacctgcggtggatcctgaccttcatgctgctcttcgtcctggtgtgtgagattgcagagggcatcctgtctgatggggtgaccgaatcccaccatctgcacctgtacatgccagccgggatggcgttcatggctgctgtcacctccgtggtctactatcacaacatcgagacttccaacttccccaagctgctaattgccctgctggtgtattggaccctggccttcatcaccaagaccatcaagtttgtcaagctcttggaccacgccatcggcttctcgcagctacgcttctgcctcacagggctgctggtgatcctctatgggatgctgctcctcgtggaggtcaatgtcatcagggtgaggagatacatcttcttcaagacaccgagggaggtgaagcctcccgaggacctgcaagacctgggggtacgcttcctgcagcccttcgtgaatctgccgtccaaaggcacctactggtggatgaacgccttcatcaagactgcccacaagaagcccatcgacttgcgagccatcgggaagctgcccatcgttatgagggccctcaccaactaccaacggctctgcgaggcctttgacgcccaggtgcggaaggacattcagggcactcaaggtgcccgggccatctggcaggcactcagccatgccttcgggaggcgcctggtcctcagcagcactttccgcatcttggccgacctgctgggcttcgccgggccactgtgcatctttgggatcgtggaccaccttgggaaggagaacgacgtcttccagcccaagacacaatttctcggggtttactttgtctcatcccaagagttccttgccaatgcctacgtcttagctgtgcttctgttccttgccctcctactgcaaaggacatttctgcaagcatcctactatgtggccattgaaactggaattaacttgagaggagcaatacagaccaagatttacaataaaattatgcacctgtccacctccaacctgtccatgggagaaatgactgctggacagatctgtaatctggttgccatcgacaccaatcagctcatgtggtttttcttcttgtgcccaaacctctgggctatgccagtacagatcattgtgggtgtgattctcctctactacatactcggagtcagtgccttaattggagcagctgtcatcattctactggctcctgtccagtacttcgtggccaccaagctgtctcaggcccagcggagcacactggagtattccaatgagcggctgaagcagaccaacgagatgctccgcggcatcaagctgctgaagctgtacgcctgggagaacatcttccgcacgcgggtggagacgacccgcaggaaggagatgaccagcctcagggcctttgccatctatacctccatctccattttcatgaacacggccatccccattgcagctgtcctcataactttcgtgggccatgtcagcttcttcaaagaggccgacttctcgccctccgtggcctttgcctccctctccctcttccatatcttggtcacaccgctgttcctgctgtccagtgtggtccgatctaccgtcaaagctctagtgagcgtgcaaaagctaagcgagttcctgtccagtgcagagatccgtgaggagcagtgtgccccccatgagcccacacctcagggcccagccagcaagtaccaggcggtgcccctcagggttgtgaaccgcaagcgtccagcccgggaggattgtcggggcctcaccggcccactgcagagcctggtccccagtgcagatggcgatgctgacaactgctgtgtccagatcatgggaggctacttcacgtggaccccagatggaatccccacactgtccaacatcaccattcgtatcccccgaggccagctgactatgatcgtggggcaggtgggctgcggcaagtcctcgctccttctagccgcactgggggagatgcagaaggtctcaggggctgtcttctggagcagccttcctgacagcgagataggagaggaccccagcccagagcgggagacagcgaccgacttggatatcaggaagagaggccccgtggcctatgcttcgcagaaaccatggctgctaaatgccactgtggaggagaacatcatctttgagagtcccttcaacaaacaacggtacaagatggtcattgaagcctgctctctgcagccagacatcgacatcctgccccatggagaccagacccagattggggaacggggcatcaacctgtctggtggtcaacgccagcgaatcagtgtggcccgagccctctaccagcacgccaacgttgtcttcttggatgaccccttctcagctctggatatccatctgagtgaccacttaatgcaggccggcatccttgagctgctccgggacgacaagaggacagtggtcttagtgacccacaagctacagtacctgccccatgcagactggatcattgccatgaaggatgggaccatccagagggagggtaccctcaaggacttccagaggtctgaatgccagctctttgagcactggaagaccctcatgaaccgacaggaccaagagctggagaaggagactgtcacagagagaaaagccacagagccaccccagggcctatctcgtgccatgtcctcgagggatggccttctgcaggatgaggaagaggaggaagaggaggcagctgagagcgaggaggatgacaacctgtcgtccatgctgcaccagcgtgctgagatcccatggcgagcctgcgccaagtacctgtcctccgccggcatcctgctcctgtcgttgctggtcttctcacagctgctcaagcacatggtcctggtggccatcgactactggctggccaagtggaccgacagcgccctgaccctgacccctgcagccaggaactgctccctcagccaggagtgcaccctcgaccagactgtctatgccatggtgttcacggtgctctgcagcctgggcattgtgctgtgcctcgtcacgtctgtcactgtggagtggacagggctgaaggtggccaagagactgcaccgcagcctgctaaaccggatcatcctagcccccatgaggttttttgagaccacgccccttgggagcatcctgaacagattttcatctgactgtaacaccatcgaccagcacatcccatccacgctggagtgcctgagccgctccaccctgctctgtgtctcagccctggccgtcatctcctatgtcacacctgtgttcctcgtggccctcttgcccctcgcagtcgtgtgctacttcatccagaagtacttccgggtggcgtccagggacctgcagcagctggatgacaccacccagcttccacttctctcacactttgccgaaaccgtagaaggactcaccaccatccgggccttcaggtatgaggcccggttccagcagaagcttctcgaatacacagactccaacaacattgcttccctcttcctcacagctgccaacagatggctggaagtccgaatggagtacatcggtgcatgtgtggtgctcatcgcagcggtgacctccatctccaactccctgcacagggagctctctgctggcctggtgggcctgggccttacctacgccctaatggtctccaactacctcaactggatggtgaggaacctggcagacatggagctccagctgggggctgtgaagcgcatccatgggctcctgaaaaccgaggcagagagctacgaggggctcctggcaccatcgctgatcccaaagaactggccagaccaagggaagatccagatccagaacctgagcgtgcgctacgacagctccctgaagccggtgctgaagcacgtcaatgccctcatctcccctggacagaagatcgggatctgcggccgcaccggcagtgggaagtcctccttctctcttgccttcttccgcatggtggacacgttcgaagggcacatcatcattgatggcattgacatccgcaaactgccgctgcacaccctgccgtcacgcctctccatcatcctgcaggaccccgtcctcttcagcggcaccatccgatttaacctggaccctgagaggaagtgctcagatagcacactgtgggaggccctggaaatcgcccagctgaagctggtggtgaaggcactgccaggaggcctcgatgccatcatcacagaaggcggggagaatttcagccagggacagaggcagctgttctgcctggcccgggccttcgtgaggaagaccagcatcttcatcatggacgaggccacggcttccattgacatggccacggaaaacatcctccaaaaggtggtgatgacagccttcgcagaccgcactgtggtcaccatcgcgcatcgagtgcacaccatcctgagtgcagacctggtgatcgtcctgaagcggggtgccatccttgagttcgataagccagagaagctgctcagccggaaggacagcgtcttcgcctccttcgtccgtgcagacaagtgacctgccagagcccaagtgccatcccacattcggaccctgcccatacccctgcctgggttttctaactgtaaatcacttgtaaataaatagatttgattatttcct

SEQ ID NO: 68 - DNA, 4746 bp, Homo sapiens
atgcccctggccttctgcggcagcgagaaccactcggccgcctaccgggtggaccagggggtcctcaacaacggctgctttgtggacgcgctcaacgtggtgccgcacgtcttcctactcttcatcaccttccccatcctcttcattggatggggaagtcagagctccaaggtgcacatccaccacagcacatggcttcatttccccgggcacaacctgcggtggatcctgaccttcatgctgctcttcgtcctggtgtgtgagattgcagagggcatcctgtctgatggggtgaccgaatcccaccatctgcacctgtacatgccagccgggatggcgttcatggctgctgtcacctccgtggtctactatcacaacatcgagacttccaacttccccaagctgctaattgccctgctggtgtattggaccctggccttcatcaccaagaccatcaagtttgtcaagctcttggaccacgccatcggcttctcgcagctacgcttctgcctcacagggctgctggtgatcctctatgggatgctgctcctcgtggaggtcaatgtcatcagggtgaggagatacatcttcttcaagacaccgagggaggtgaagcctcccgaggacctgcaagacctgggggtacgcttcctgcagcccttcgtgaatctgccgtccaaaggcacctactggtggatgaacgccttcatcaagactgcccacaagaagcccatcgacttgcgagccatcgggaagctgcccatcgttatgagggccctcaccaactaccaacggctctgcgaggcctttgacgcccaggtgcggaaggacattcagggcactcaaggtgcccgggccatctggcaggcactcagccatgccttcgggaggcgcctggtcctcagcagcactttccgcatcttggccgacctgctgggcttcgccgggccactgtgcatctttgggatcgtggaccaccttgggaaggagaacgacgtcttccagcccaagacacaatttctcggggtttactttgtctcatcccaagagttccttgccaatgcctacgtcttagctgtgcttctgttccttgccctcctactgcaaaggacatttctgcaagcatcctactatgtggccattgaaactggaattaacttgagaggagcaatacagaccaagatttacaataaaattatgcacctgtccacctccaacctgtccatgggagaaatgactgctggacagatctgtaatctggttgccatcgacaccaatcagctcatgtggtttttcttcttgtgcccaaacctctgggctatgccagtacagatcattgtgggtgtgattctcctctactacatactcggagtcagtgccttaattggagcagctgtcatcattctactggctcctgtccagtacttcgtggccaccaagctgtctcaggcccagcggagcacactggagtattccaatgagcggctgaagcagaccaacgagatgctccgcggcatcaagctgctgaagctgtacgcctgggagaacatcttccgcacgcgggtggagacgacccgcaggaaggagatgaccagcctcagggcctttgccatctatacctccatctccattttcatgaacacggccatccccattgcagctgtcctcataactttcgtgggccatgtcagcttcttcaaagaggccgacttctcgccctccgtggcctttgcctccctctccctcttccatatcttggtcacaccgctgttcctgctgtccagtgtggtccgatctaccgtcaaagctctagtgagcgtgcaaaagctaagcgagttcctgtccagtgcagagatccgtgaggagcagtgtgccccccatgagcccacacctcagggcccagccagcaagtaccaggcggtgcccctcagggttgtgaaccgcaagcgtccagcccgggaggattgtcggggcctcaccggcccactgcagagcctggtccccagtgcagatggcgatgctgacaactgctgtgtccagatcatgggaggctacttcacgtggaccccagatggaatccccacactgtccaacatcaccattcgtatcccccgaggccagctgactatgatcgtggggcaggtgggctgcggcaagtcctcgctccttctagccgcactgggggagatgcagaaggtctcaggggctgtcttctggagcagccttcctgacagcgagataggagaggaccccagcccagagcgggagacagcgaccgacttggatatcaggaagagaggccccgtggcctatgcttcgcagaaaccatggctgctaaatgccactgtggaggagaacatcatctttgagagtcccttcaacaaacaacggtacaagatggtcattgaagcctgctctctgcagccagacatcgacatcctgccccatggagaccagacccagattggggaacggggcatcaacctgtctggtggtcaacgccagcgaatcagtgtggcccgagccctctaccagcacgccaacgttgtcttcttggatgaccccttctcagctctggatatccatctgagtgaccacttaatgcaggccggcatccttgagctgctccgggacgacaagaggacagtggtcttagtgacccacaagctacagtacctgccccatgcagactggatcattgccatgaaggatgggaccatccagagggagggtaccctcaaggacttccagaggtctgaatgccagctctttgagcactggaagaccctcatgaaccgacaggaccaagagctggagaaggagactgtcacagagagaaaagccacagagccaccccagggcctatctcgtgccatgtcctcgagggatggccttctgcaggatgaggaagaggaggaagaggaggcagctgagagcgaggaggatgacaacctgtcgtccatgctgcaccagcgtgctgagatcccatggcgagcctgcgccaagtacctgtcctccgccggcatcctgctcctgtcgttgctggtcttctcacagctgctcaagcacatggtcctggtggccatcgactactggctggccaagtggaccgacagcgccctgaccctgacccctgcagccaggaactgctccctcagccaggagtgcaccctcgaccagactgtctatgccatggtgttcacggtgctctgcagcctgggcattgtgctgtgcctcgtcacgtctgtcactgtggagtggacagggctgaaggtggccaagagactgcaccgcagcctgctaaaccggatcatcctagcccccatgaggttttttgagaccacgccccttgggagcatcctgaacagattttcatctgactgtaacaccatcgaccagcacatcccatccacgctggagtgcctgagccgctccaccctgctctgtgtctcagccctggccgtcatctcctatgtcacacctgtgttcctcgtggccctcttgcccctcgcagtcgtgtgctacttcatccagaagtacttccgggtggcgtccagggacctgcagcagctggatgacaccacccagcttccacttctctcacactttgccgaaaccgtagaaggactcaccaccatccgggccttcaggtatgaggcccggttccagcagaagcttctcgaatacacagactccaacaacattgcttccctcttcctcacagctgccaacagatggctggaagtccgaatggagtacatcggtgcatgtgtggtgctcatcgcagcggtgacctccatctccaactccctgcacagggagctctctgctggcctggtgggcctgggccttacctacgccctaatggtctccaactacctcaactggatggtgaggaacctggcagacatggagctccagctgggggctgtgaagcgcatccatgggctcctgaaaaccgaggcagagagctacgaggggctcctggcaccatcgctgatcccaaagaactggccagaccaagggaagatccagatccagaacctgagcgtgcgctacgacagctccctgaagccggtgctgaagcacgtcaatgccctcatctcccctggacagaagatcgggatctgcggccgcaccggcagtgggaagtcctccttctctcttgccttcttccgcatggtggacacgttcgaagggcacatcatcattgatggcattgacatccgcaaactgccgctgcacaccctgccgtcacgcctctccatcatcctgcaggaccccgtcctcttcagcggcaccatccgatttaacctggaccctgagaggaagtgctcagatagcacactgtgggaggccctggaaatcgcccagctgaagctggtggtgaaggcactgccaggaggcctcgatgccatcatcacagaaggcggggagaatttcagccagggacagaggcagctgttctgcctggcccgggccttcgtgaggaagaccagcatcttcatcatggacgaggccacggcttccattgacatggccacggaaaacatcctccaaaaggtggtgatgacagccttcgcagaccgcactgtggtcaccatcgcgcatcgagtgcacaccatcctgagtgcagacctggtgatcgtcctgaagcggggtgccatccttgagttcgataagccagagaagctgctcagccggaaggacagcgtcttcgcctccttcgtccgtgcagacaagtga

SEQ ID NO: 69 - protein, 1581 aa, Homo sapiens
MPLAFCGSENHSAAYRVDQGVLNNGCFVDALNVVPHVFLLFITFPILFIGWGSQSSKVHIHHSTWLHFPGHNLRWILTFMLLFVLVCEIAEGILSDGVTESHHLHLYMPAGMAFMAAVTSVVYYHNIETSNFPKLLIALLVYWTLAFITKTIKFVKLLDHAIGFSQLRFCLTGLLVILYGMLLLVEVNVIRVRRYIFFKTPREVKPPEDLQDLGVRFLQPFVNLPSKGTYWWMNAFIKTAHKKPIDLRAIGKLPIVMRALTNYQRLCEAFDAQVRKDIQGTQGARAIWQALSHAFGRRLVLSSTFRILADLLGFAGPLCIFGIVDHLGKENDVFQPKTQFLGVYFVSSQEFLANAYVLAVLLFLALLLQRTFLQASYYVAIETGINLRGAIQTKIYNKIMHLSTSNLSMGEMTAGQICNLVAIDTNQLMWFFFLCPNLWAMPVQIIVGVILLYYILGVSALIGAAVIILLAPVQYFVATKLSQAQRSTLEYSNERLKQTNEMLRGIKLLKLYAWENIFRTRVETTRRKEMTSLRAFAIYTSISIFMNTAIPIAAVLITFVGHVSFFKEADFSPSVAFASLSLFHILVTPLFLLSSVVRSTVKALVSVQKLSEFLSSAEIREEQCAPHEPTPQGPASKYQAVPLRVVNRKRPAREDCRGLTGPLQSLVPSADGDADNCCVQIMGGYFTWTPDGIPTLSNITIRIPRGQLTMIVGQVGCGKSSLLLAALGEMQKVSGAVFWSSLPDSEIGEDPSPERETATDLDIRKRGPVAYASQKPWLLNATVEENIIFESPFNKQRYKMVIEACSLQPDIDILPHGDQTQIGERGINLSGGQRQRISVARALYQHANVVFLDDPFSALDIHLSDHLMQAGILELLRDDKRTVVLVTHKLQYLPHADWIIAMKDGTIQREGTLKDFQRSECQLFEHWKTLMNRQDQELEKETVTERKATEPPQGLSRAMSSRDGLLQDEEEEEEEAAESEEDDNLSSMLHQRAEIPWRACAKYLSSAGILLLSLLVFSQLLKHMVLVAIDYWLAKWTDSALTLTPAARNCSLSQECTLDQTVYAMVFTVLCSLGIVLCLVTSVTVEWTGLKVAKRLHRSLLNRIILAPMRFFETTPLGSILNRFSSDCNTIDQHIPSTLECLSRSTLLCVSALAVISYVTPVFLVALLPLAVVCYFIQKYFRVASRDLQQLDDTTQLPLLSHFAETVEGLTTIRAFRYEARFQQKLLEYTDSNNIASLFLTAANRWLEVRMEYIGACVVLIAAVTSISNSLHRELSAGLVGLGLTYALMVSNYLNWMVRNLADMELQLGAVKRIHGLLKTEAESYEGLLAPSLIPKNWPDQGKIQIQNLSVRYDSSLKPVLKHVNALISPGQKIGICGRTGSGKSSFSLAFFRMVDTFEGHIIIDGIDIRKLPLHTLPSRLSIILQDPVLFSGTIRFNLDPERKCSDSTLWEALEIAQLKLVVKALPGGLDAIITEGGENFSQGQRQLFCLARAFVRKTSIFIMDEATASIDMATENILQKVVMTAFADRTVVTIAHRVHTILSADLVIVLKRGAILEFDKPEKLLSRKDSVFASFVRADK

SEQ ID NO: 70 - DNA, 6914 bp, Homo sapiens
tcattgcagctgggacagcccggagtgtggttagcagctcggcaagcgctgcccaggtcctggggtggtggcagccagcgggagcaggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcagagaagatggacgacgctgtggctccaggccgagaatacacctatgaatggagtatcagtgaggacagtggacccacccatgatgaccctccatgcctcacacacatctattactcccatgaaaatctgatcgaggatttcaactctgggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaattaggtcattccgaaactcatcattgaaccaggaagaagaagagttcaatcttactgccctagctctggagaatggcactgaattcgtttcttcgaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcagaagtcaagaacatgctaagcgtaagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctgggaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttctccagccctcggtcagatgcccctttctccagacctcagccatacaaccatttctctagacttcagccagacaaacctctctccagaactcagccatatgactctctctccagaactcagtcagacaaacctttccccagccctcggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctgtggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactagaattgaacattcaaaaacccctggaagagactctttaagacctcaaaccatttagaatgggcaatgtattttacgctgtgttaaatgttaacagttttccactatttctctttcttttctattagtgaataaaattttatac

SEQ ID NO: 71 - DNA, 6675 bp, Homo sapiens
atgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcagagaagatggacgacgctgtggctccaggccgagaatacacctatgaatggagtatcagtgaggacagtggacccacccatgatgaccctccatgcctcacacacatctattactcccatgaaaatctgatcgaggatttcaactctgggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaattaggtcattccgaaactcatcattgaaccaggaagaagaagagttcaatcttactgccctagctctggagaatggcactgaattcgtttcttcgaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcagaagtcaagaacatgctaagcgtaagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctgggaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttctccagccctcggtcagatgcccctttctccagacctcagccatacaaccatttctctagacttcagccagacaaacctctctccagaactcagccatatgactctctctccagaactcagtcagacaaacctttccccagccctcggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctgtggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactag

SEQ ID NO: 72 - protein, 2224 aa, Homo sapiens
MFPGCPRLWVLVVLGTSWVGWGSQGTEAAQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDPLQPDVTGIRLLSLGAGEFRSQEHAKRKGPKVERDQAAKHRFSWMKLLAHKVGRHLSQDTGSPSGMRPWEDLPSQDTGSPSRMRPWEDPPSDLLLLKQSNSSKILVGRWHLASEKGSYEIIQDTDEDTAVNNWLISPQNASRAWGESTPLANKPGKQSGHPKFPRVRHKSLQVRQDGGKSRLKKSQFLIKTRKKKKEKHTHHAPLSPRTFHPLRSEAYNTFSERRLKHSLVLHKSNETSLPTDLNQTLPSMDFGWIASLPDHNQNSSNDTGQASCPPGLYQTVPPEEHYQTFPIQDPDQMHSTSDPSHRSSSPELSEMLEYDRSHKSFPTDISQMSPSSEHEVWQTVISPDLSQVTLSPELSQTNLSPDLSHTTLSPELIQRNLSPALGQMPISPDLSHTTLSPDLSHTTLSLDLSQTNLSPELSQTNLSPALGQMPLSPDLSHTTISLDFSQTNLSPELSHMTLSPELSQTNLSPALGQMPISPDLSHTTLSLDFSQTNLSPELSQTNLSPALGQMPLSPDPSHTTLSLDLSQTNLSPELSQTNLSPDLSEMPLFADLSQIPLTPDLDQMTLSPDLGETDLSPNFGQMSLSPDLSQVTLSPDISDTTLLPDLSQISPPPDLDQIFYPSESSQSLLLQEFNESFPYPDLGQMPSPSSPTLNDTFLSKEFNPLVIVGLSKDGTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPVDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 73 - DNA, 3481 bp, Homo sapiens
aagtaacgccgcggaccgggaaagtgggaggggccgctcccggaacgcagccttcttgtaagaacctccaaggaagcaagaagaaaaaagaggcgctaccctgcgctcccgggaggcctctgaaagcttccactagagaaaaactccccactcttacaatttctttaaccgcaagaagcggaggacctggacaaggactcgaggagcaaggtggcgaaccaagggtagggcgcaccgggcccgagaggtcccccgcaggttgcagatacggtggactctctgcggcttctgagcacggagggagctgtcgcggggtcgggaggtcgtctctcatccctgcctcatcttccgacggccggcggggccatgtccagaggcccgagctccgcggtcctgcctagcgccctgggatcccggaagctcggcccccggagcctcagctgcctgtcggacctggacggcggcgtggccctggagccgcgggcctgtaggccccctgggagcccgggccgcgcgccgccgccaacgccagcgccgtcgggctgcgacccccgcctgcggcccatcatcctgcggcgggcgcgctcactgcccagctcccccgagcgccgccagaaggccgcgggcgcgccgggcgctgcgtgtcggccgggctgcagccagaagctccgcgtgcgcttcgccgacgccctgggcttggagctggcacaggtcaaggtgttcaacgcgggagacgacccgtccgtgccgctgcacgtgctgtcgcggctcgcaatcaactcggacctgtgctgcagcagccaggacctggagttcaccctgcattgcctggtgcccgatttcccgccgcccgtcgaggccgccgactttggcgagcgcctgcagcggcagctcgtgtgcctggagcgtgtcacttgctcggaccttggcatcagcggtacggtgcgcgtgtgcaacgtggccttcgagaagcaggtggctgtgcgctacactttctcgggctggcgcagtacccacgaggcggtggcgcggtggcgcgggcccgcaggccccgagggcacggaggacgttttcaccttcggctttccagtaccgcccttcctgctggagctcggctcccgcgtgcacttcgcggtgcgctaccaagtggcgggtgccgagtactgggacaacaacgaccaccgagactacagcctcacatgtcgcaaccacgcgctgcacatgcctcgcggggagtgcgaagagagctggatccacttcatctgagccgcgcggggaccggccacctggagcctccacacctaagctgcgcctcctgtcatttccctgctgggctctcacatctatctggttgttcttcaccaccctccaagtcctctgacctaattttctgctgcaaggtcccctggcagtggcccatcctgtcttctacttgaaacgtctgagtcacttggtctaaaaagtagcctcaggtggccagaaggccgagttgtgtaatgagttggggcagggtggtggggtaggtgcatgggagggtgggccctctggggaggcccaagcagcttgttttgcaaaggcccaagtcctcctgctaggaaaagcttttgcatgtgtcctgaatgtgtctctgtaaacatagctgttatttattattgtgatgttgggacctttagcccatagcggatgcctcctcaggaatattctcgatttaataagctaaaaaaaaaaagttctgtgtttgtgcccacagtaaacagaatgtacagtgtgacccacatcgtggtccctccaggcattcctgccccaggataaggaaaacctgagcccacatctgattgccaacggctgtagccttctaacagtgggaaactattaataggcaagaaatttctgctttatcagtcatcacacaaccattcctgggcattgagactgtcctggttaggctgtgtgtctcctacattaacagctggggaggagggaaaagcttcagacttttaaactagcaaaacagttatttctctgcattcacaattctgatttcatgtatttctctgtacatgtttgatcattatcaggccattctgtttaagtttgaaactaagcattttaagaagtcttgcctcagttatttcagttttcaggtgactagagaatttcaggcaacagtgccctttgcaattacctttttgatgattaaggtgactatttttatttcttgttaagcatcagaagatgtcagcattcatcttttgggagtccagttacaaggtggaactagaaggtgttttcagcaatagccagagcactggaggaaaaataattgacaaatatttatacttttaaattgttttgaactgtatttacatacatcatgttttatcaggtacccaccaactttatactttctgccttcagatagagtttctaacattttctagtttgtcagttttaaaatataaaattcttttatgtttttaaaccgtcaaccaagaagtcaagaccactgtcccctgctgagttaggcaatcacttaaaactccaaagaactccaggctgaggagttcttttggtgctgaacagtggtctgacaacaggtgctagaattaaggtgtgggaaaaaaaaaaaaacagccaggaaggacaagatctccaatttgtgaagcttatgatacagctgaaaccagtctgatcaattttatgctacctgtcaccaagaaattaactctaaatttttgaatgcttgttggcatctgtcaaatcaatactgaaaggatgcatttcggcttttaaccagtttggttctgatcattgcaacctctgtgcttttataattcaaagaacaaggatagctttcttcggtatcttttaatataagaacactgaattttggcttgaaatattgtgactaaattgcacaatctctggctattagcactgttcactacattaacatgcaagagagagagaagccttgttacatttcctgctatttaacaaactgtccaattaggtcagcaagcctgttagggccttcactgctacgcccctggccccaaaacagagagcaagacagttgtccagagaatttagatatgagttaagagctgccagatttattttaaggcatgtagagaatctcataatctatgtgaaagtcacttgccaaaaaggttttggttatgaaagtgaatgttatatatttaatattaagtgaacctcccgtgccttttaaaaaataccaacattatgaaatttaaaagcttaaatatatttgcctagttgataaagaggctgtggaacaactgaaggagaactttcagaccctaaaacagttatattacttagatgttaaaattttctagtgggctgtgcaattgctgggttgtccccccaaacatattttaatatgttatttaaaatgattttacccaagcctaagtattaaagtatatgtgcaaaaacaaaaaaaaaaaaaaaa

SEQ ID NO: 74 - DNA, 900 bp, Homo sapiens
atgtccagaggcccgagctccgcggtcctgcctagcgccctgggatcccggaagctcggcccccggagcctcagctgcctgtcggacctggacggcggcgtggccctggagccgcgggcctgtaggccccctgggagcccgggccgcgcgccgccgccaacgccagcgccgtcgggctgcgacccccgcctgcggcccatcatcctgcggcgggcgcgctcactgcccagctcccccgagcgccgccagaaggccgcgggcgcgccgggcgctgcgtgtcggccgggctgcagccagaagctccgcgtgcgcttcgccgacgccctgggcttggagctggcacaggtcaaggtgttcaacgcgggagacgacccgtccgtgccgctgcacgtgctgtcgcggctcgcaatcaactcggacctgtgctgcagcagccaggacctggagttcaccctgcattgcctggtgcccgatttcccgccgcccgtcgaggccgccgactttggcgagcgcctgcagcggcagctcgtgtgcctggagcgtgtcacttgctcggaccttggcatcagcggtacggtgcgcgtgtgcaacgtggccttcgagaagcaggtggctgtgcgctacactttctcgggctggcgcagtacccacgaggcggtggcgcggtggcgcgggcccgcaggccccgagggcacggaggacgttttcaccttcggctttccagtaccgcccttcctgctggagctcggctcccgcgtgcacttcgcggtgcgctaccaagtggcgggtgccgagtactgggacaacaacgaccaccgagactacagcctcacatgtcgcaaccacgcgctgcacatgcctcgcggggagtgcgaagagagctggatccacttcatctga

SEQ ID NO: 75 - protein, 299 aa, Homo sapiens
MSRGPSSAVLPSALGSRKLGPRSLSCLSDLDGGVALEPRACRPPGSPGRAPPPTPAPSGCDPRLRPIILRRARSLPSSPERRQKAAGAPGAACRPGCSQKLRVRFADALGLELAQVKVFNAGDDPSVPLHVLSRLAINSDLCCSSQDLEFTLHCLVPDFPPPVEAADFGERLQRQLVCLERVTCSDLGISGTVRVCNVAFEKQVAVRYTFSGWRSTHEAVARWRGPAGPEGTEDVFTFGFPVPPFLLELGSRVHFAVRYQVAGAEYWDNNDHRDYSLTCRNHALHMPRGECEESWIHFI

SEQ ID NO: 76 - DNA, 3411 bp, Homo sapiens
gggcgagtgacagccccggctccgcgcgccgcggccgccagagccggcgcaggggaagcgcccgcggccccgggtgcagcagcggccgccgcctcccgcgcctccccggcccgcagcccgcggtcccgcggccccggggccggcacctctcgggctccggctccccgcgcgcaagatggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttcgaccgatttttcacccgccatccaccagtcctaacacctcccgaccaggaagtcatcaggaatattgaccaatcagaattcgaaggattttcctttgttaactctgaatttttaaaacccgaagtcaagagctaagtagatgtgtagatctccgtccttcatttctgtcattcaagctcaacggctattgtggtgacatttttatgtttttcattgccaagttgcatccatgtttgattttctgatgagactagagtgacagtgtttcagaacccaaatgtcctcaggtagtttggagcatctctatgagatgggattatgcagatggcctatggaaaatgcagctgcataattaacacattatcaaagtcctcttacaatttattttccgcagcatgtcagctaagtagacccaatggggagagaaaatgcctgctttctttccctctttttctgcactgccatattcacccccaaccatccaatctgtggataattggatgttagcggtactcttccacttccgggcctggagcttggcttgtatccaagtgtatggttgctttgcctaagaggaatccctctatttcacctgttctggaggcaccagaccttgaaaagaacatgctcaaaataaaatgttatctgttatttttgtaaactcaaagttaagatgatcaaagttctaaaattccaagaatgtgcttttagacggtctcaatctaaaagcacttcaaggggtcaaagggcaaccagcttgggtgctacctcagtgttgtagtttctgatactttatgtctttgctcaccctcatccccaaactacttgaaaagggcatttggcaccactctctgaaacaacacagtcactctagcaaggcccccaaagggccctggttttacattacatttcaaactttatttgctttggggttttgtttctgttgttgttcaaatgcaaaaaaaagaaaaaaaaagaaaaaaaaaggtgactcacattgttacacatgctttaaaatatgtattcaaatgttattaaccacaatgacgacctgctttgatttaaccaagaagacggctgcggagcctagcagactcaggcctgtgggaatgggatttgttacaaatctaggtttgttactggcttcagaaagctaattaagtgctctgaaaaagacaccgtttcttgaaacaaagatggttgtattcctcactttgatgttgttttgcaagatgtttgtggaaatgttcatttgtatctggatctctgttatgtgccatttttcttctagcatcgagatacaataaaaaaaaaaaaaaagaaaaaaaaaaaaaaaaaaaaaaaaaaaa

SEQ ID NO: 77 - DNA, 2022 bp, Homo sapiens
atggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttcgaccgatttttcacccgccatccaccagtcctaacacctcccgaccaggaagtcatcaggaatattgaccaatcagaattcgaaggattttcctttgttaactctgaatttttaaaacccgaagtcaagagctaa

SEQ ID NO: 78 - protein, 673 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFDRFFTRHPPVLTPPDQEVIRNIDQSEFEGFSFVNSEFLKPEVKS

SEQ ID NO: 79 - DNA, 5760 bp, Homo sapiens
actcattgtgtctgtgtcgaggcgtcgggagggcctaagtccgtgtgcggtgcccttcggccggcctgagccccagagtcagctcccctttctcgcccagcgcccccaggccgctcccggggctcacggaatagtaaagaaacacatcataaaacctcccaggacataaaggtgagcacagaccctgtttggatcaagtcagttcctggagcctgaatgatgactgctgaatcacgggaagccacgggtctgtccccacaggctgcacaggagaaggatggtatcgtaatagtgaaggtggaagaggaagatgaggaagaccacatgtgggggcaggattccaccctacaggacacgcctcctccagacccagagatattccgccaacgcttcaggcgcttctgttaccagaacacttttgggccccgagaggctctcagtcggctgaaggaactttgtcatcagtggctgcggccggaaataaacaccaaggaacagatcctggagcttctggtgctagagcagtttctttccatcctgcccaaggagctccaggtctggctgcaggaataccgccccgatagtggagaggaggccgtgacccttctagaagacttggagcttgatttatcaggacaacaggtcccaggtcaagttcatggacctgagatgctcgcaagggggatggtgcctctggatccagttcaggagtcctcgagctttgaccttcatcacgaggccacccagtcccacttcaaacattcgtctcggaaaccccgcctcttacagtcacgagctcttcctgctgcccacattcctgcaccccctcatgagggtagtcccagagaccaggcgatggcatctgcactattcacagcggattcccaggcaatggtgaagatcgaggacatggctgtgtccctcattctggaggaatggggatgtcagaatctggctcggaggaatctcagtagggacaacaggcaggagaattatgggagcgcatttccccagggtggtgaaaacaggaatgagaacgaggagtcaacctcaaaggctgaaacctcggaagattcagcatcacgcggggagacaacaggaagatcccagaaagagtttggagagaaacgtgaccaggagggcaaaacaggagaaagacagcagaaaaaccctgaggagaaaaccaggaaagagaaaagagattcagggccagctataggaaaggacaaaaaaaccatcacaggagagagaggtccaagggagaaggggaaaggattgggaagaagcttcagtctgagctccaacttcaccacccctgaagaagttcccacgggaacaaagtctcacagatgtgatgaatgtggtaaatgcttcacgagaagttcaagccttatccgccataaaataatccacactggagaaaagccctatgaatgtagtgagtgtgggaaagccttcagtcttaactccaaccttgtcctgcatcagaggatccacacaggagagaaacctcatgaatgtaacgagtgtggcaaggccttcagccacagttccaatctcatcctccatcagcgcatccactctggagagaaaccttatgaatgtaatgagtgcgggaaggccttcagccagagctctgacctcaccaagcatcagagaattcacacgggggagaaaccctatgaatgtagtgaatgtggaaaagctttcaaccgaaactcatacctgattttgcatcggagaattcacactcgagaaaagccctacaagtgcactaagtgtggcaaggccttcacccgcagctccaccctcactctgcatcacagaatccatgccagagagagagcctctgagtacagcccagcctcccttgatgcatttggcgcgttcctgaaaagttgtgtgtaaaggaagaatttgccatcaagccatttcccccttttgtttctaaaattatttcagagatgtgtgctcctggagggaaaaagaaatacagcctcaacagattaaaaaacaaaagtcacacttaaggacccttctagtcacatcagcagtgttctgcctttatgtagtagttgggcatataatccttccacacagcccctgcagggaaaggctaatcttacggataatccacgtgagatttccacacaagagaaaagcacacgcatagtgaaatgtcagtcttttcagtaatgaggatacctttaaggcactcttggactctcggcaaccacaacataatagttgaaagatcaagattggctccacgaaagtgatacggaggttaggatgctacttgctgcaaacaagccctactttggccaacatcctgcttatttctcaaaaaagagggacagtgaaaacaaaaacgacattgggacatgctgctcaaggtagttatatatacgataagttgtatatatgatcactggtagcctaccaaagctgtagaaatctaggactgtgctaatcagtatcaaaccaaagatttctatctcttcccgaaagagagggtatgtgcaccagtctacagttccaaaggactgcaacaaatgtagatggttctgtcctcatccctgagatcagttctactgaaatggcaacaacaactccaaatacatctctcccttcttgaaatccctaaagcactatcgcactcctaaatgcatttctccacaagttagcacttgattgtatactgtcttttaatccttcattgtttcatgtatgaagtctttaatcaccccctaacccccaaaaaagattcggttgtcaagggatatatttctatccctgccagcacagtgctggacatagtgagtactgaacacctaagaggcactcattcagactgacttctacaggacttctacgtgtgtgataaaagcctgtgaatcgtgagtccctgaaatatggtagcctggcccagcttctaaagaggaccttcgtagccacaaggcatgctaaacctctagggtctgatgctgtttttgttccaaatataaacgaaaggcactagtcagccgcctgcatagactttctaaacagtgagtaaataccatggaatgtcagaaatgactttatcatcgtcatcttgaagaaaaatatgaacactgatgaaggtcattttttttttttgacatctttctgtggcaaagctcttagaattctttgcagttagctgctgaacagtatgatgatttggggattttctgaatcatttgtacttagatactggagttcagaagatgtgatttttgttgcttttcagaaaaaggaacgtggtagggagtgttttttcccacgcttttccagtaactttgcaaaagcattaattgttcactggcaattactaaatgtattttgttgctctgagaaactcagcagtgtactgagatgtggctggctgtgccatcgtcatagtgcacagtgacttttctgtttcttatcactaaagactgaggtgaggttatgaaactttcattggtcccatcgttgtgcgcagggtgcaactggctacctagaagctgatggcaggagactgtttcacacacaggagattgtgagctgtgtaagtagtcatcgccactcaagtaggacaagggtcctacccaaggctaggaccgccctgcggaaacagaaacatagactgagaacaaacctcaagactatcagtgtgacctccccataacaagagaaccccatatatagtttgagactttcccttgagaaacttatactaaaactattactcatcagtcattcactgatcagcagctaaagtccatgagaccaaatggttttatatggaaccaacaaagtgggagctaaaactgcacagtggtcattctttggcctctccttggctttatgacttaaaccaactacaacttccctatagcttctaagcagtttcatcagcattacttgggaaaacgtgttgcaagtcaaccagtcactaggatatttctacccatgcaacggaagaaaaacccattactccagaagagtatatctaagtaactaaaatggaggcagaaaatgttttaaggattttcttcaaagaataagccacagcaatggttttgtagaaaactcctgtgcttttgagcaaggacttttgccctctagaaagcaactgaggccaggtgcggtggctcacgcctgtaatcccagcactttgggaggccgaggtgagcggatcacctgaggtcaggagttttgagaccagcctggccaacatggtaaaatcccgtctctactaaaattataaaaattagccgggcatgatggtgggcacctgtaatcccagctacttgggaggctgaggcaggagaatcacttgaacccaggaggcagaggttgcagtgagccaagattgcacactgcactccagcctgggtgactagcaaaactccatctcaaaaaaaaaaaaagaaagaaagaaactgaaaagtgaggtgtggaactccaggcaacgacccaagcacttattttttaagagggaaaggactttggtcatgtttacattggcctttggttttttttttttctttcagccacaactatatgctgatataactcagccatcacttttgagttttgtttttaaatgaaggttaaatgtgacctgtgccctcacatttaattccgtttcatggagacatcttcctgactttgttggatagctgccactagggttgcttcgatcttcagccacatgctgtccttgaagcagctatttgaagatgtgttttctgaggaaaacaggctacaactgtttattaaaaccacttgcagtgcattcgtttatcagatgctcagtgcaaagtgtaattgggtcatggtcaaaaacgcttgcaacatctaattggcgttcaagatagtcattcaaaagttcttggcccgctgaagtctgttaacagttgaaacattctacagttaaccattagcatgctagttgtcctaatggaaatttttgaagagtttttcaatatataccctacccttgccaggaagagaagtaaaatcctcaggttgtggggtatttgtttctactccagcctggcaggcaaggctaaaacctgagattatcgatctatgagacatcttgatatgtaaagcacttaatattaaaggcataaaagtgaaactgctaaatatgtatagagaggttgacgtgtgatacgtgggacagttcacatagacatcagagaatttattccagaaaggagcctcctgaatgtgatgaatacggcaaagcctttaatcacatctcagcccttagcatcggaaagcttatactgtaaataaacttgatgaatattatatgtgaggaaaactttcatgtatagcactcattgcttcagacagaaaatgaattccgtcggtatgttccaatcgtgatgaatttgagaaacattgcaagagggagctcaatcttggccgggcgcagtggctcacgcctgtaatcgcagcactttgggaggcagaggtgggcggatcacttgaggtcaggagtttgagaccagcctggccaacgtggtgaaagcctgtctctactaaaaatacaaaaattagccgggcgtggtggtgggcgcctgtagtcccagctactcaggaggctgaggcaggagaatcacctaaatctgggaggcagaggttgcagtgagccgagattgcaccactgcactccagcctgggtaacagagactctgtctcaaaaaaaaaaaaaaagaaaaaaaaaaaaaaaaaaaa

SEQ ID NO: 80 - DNA, 1692 bp, Homo sapiens
atgatgactgctgaatcacgggaagccacgggtctgtccccacaggctgcacaggagaaggatggtatcgtaatagtgaaggtggaagaggaagatgaggaagaccacatgtgggggcaggattccaccctacaggacacgcctcctccagacccagagatattccgccaacgcttcaggcgcttctgttaccagaacacttttgggccccgagaggctctcagtcggctgaaggaactttgtcatcagtggctgcggccggaaataaacaccaaggaacagatcctggagcttctggtgctagagcagtttctttccatcctgcccaaggagctccaggtctggctgcaggaataccgccccgatagtggagaggaggccgtgacccttctagaagacttggagcttgatttatcaggacaacaggtcccaggtcaagttcatggacctgagatgctcgcaagggggatggtgcctctggatccagttcaggagtcctcgagctttgaccttcatcacgaggccacccagtcccacttcaaacattcgtctcggaaaccccgcctcttacagtcacgagctcttcctgctgcccacattcctgcaccccctcatgagggtagtcccagagaccaggcgatggcatctgcactattcacagcggattcccaggcaatggtgaagatcgaggacatggctgtgtccctcattctggaggaatggggatgtcagaatctggctcggaggaatctcagtagggacaacaggcaggagaattatgggagcgcatttccccagggtggtgaaaacaggaatgagaacgaggagtcaacctcaaaggctgaaacctcggaagattcagcatcacgcggggagacaacaggaagatcccagaaagagtttggagagaaacgtgaccaggagggcaaaacaggagaaagacagcagaaaaaccctgaggagaaaaccaggaaagagaaaagagattcagggccagctataggaaaggacaaaaaaaccatcacaggagagagaggtccaagggagaaggggaaaggattgggaagaagcttcagtctgagctccaacttcaccacccctgaagaagttcccacgggaacaaagtctcacagatgtgatgaatgtggtaaatgcttcacgagaagttcaagccttatccgccataaaataatccacactggagaaaagccctatgaatgtagtgagtgtgggaaagccttcagtcttaactccaaccttgtcctgcatcagaggatccacacaggagagaaacctcatgaatgtaacgagtgtggcaaggccttcagccacagttccaatctcatcctccatcagcgcatccactctggagagaaaccttatgaatgtaatgagtgcgggaaggccttcagccagagctctgacctcaccaagcatcagagaattcacacgggggagaaaccctatgaatgtagtgaatgtggaaaagctttcaaccgaaactcatacctgattttgcatcggagaattcacactcgagaaaagccctacaagtgcactaagtgtggcaaggccttcacccgcagctccaccctcactctgcatcacagaatccatgccagagagagagcctctgagtacagcccagcctcccttgatgcatttggcgcgttcctgaaaagttgtgtgtaa

SEQ ID NO: 81 - protein, 563 aa, Homo sapiens
MMTAESREATGLSPQAAQEKDGIVIVKVEEEDEEDHMWGQDSTLQDTPPPDPEIFRQRFRRFCYQNTFGPREALSRLKELCHQWLRPEINTKEQILELLVLEQFLSILPKELQVWLQEYRPDSGEEAVTLLEDLELDLSGQQVPGQVHGPEMLARGMVPLDPVQESSSFDLHHEATQSHFKHSSRKPRLLQSRALPAAHIPAPPHEGSPRDQAMASALFTADSQAMVKIEDMAVSLILEEWGCQNLARRNLSRDNRQENYGSAFPQGGENRNENEESTSKAETSEDSASRGETTGRSQKEFGEKRDQEGKTGERQQKNPEEKTRKEKRDSGPAIGKDKKTITGERGPREKGKGLGRSFSLSSNFTTPEEVPTGTKSHRCDECGKCFTRSSSLIRHKIIHTGEKPYECSECGKAFSLNSNLVLHQRIHTGEKPHECNECGKAFSHSSNLILHQRIHSGEKPYECNECGKAFSQSSDLTKHQRIHTGEKPYECSECGKAFNRNSYLILHRRIHTREKPYKCTKCGKAFTRSSTLTLHHRIHARERASEYSPASLDAFGAFLKSCV

SEQ ID NO: 82 - DNA, 4244 bp, Homo sapiens
tttaaactgtgctttctaagcacagtcaggtagcaaaagtaataaaaaggatggttgaacaagttttcttgtatgttccaggatatgtttgggacttttctttgtttattatatgagttgttccctttgaaattaaagctattttgtaggttttgtgggacataatttgataagtagagttaattaaatttcttctggaagagatctaaattcttattcttagtgagagactgtagttaaaggaaggcttttagaacttgggttcaaggaagatggagatgcgtcggaagctctttggcgggggtgaggaagttcagaaagtgtgcattttccttctggcatttaggtcttgtccgtgtgatttggtggtgcttgggtcataagcctgattaaaattcagggacatgtaccacggcggccaaagcggaattaatttttttatatggggactggagcgctgaaaagttgttcctgaccaggctctaatgagaaattcctctctccccaggttatgaagacagtatggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataattttatatatatataaataatatatatatatatacatatatataaatagatctctatatagttgtggtacggtctaaaagcagtcttgtttcctggaaataaaaagttgggatattaacttgtttttgcactttagaatagcatgagaatctcactaatttagcattctgataaaagaaactttagagcaagtcagaatagagaggtgtttttcctttgaggggataggggaagtaagccaataagaaccttttaaacaaatcgtcctgtcacaaaatgctttcatatggcttaattttgtcaacactgcattgtcttttgagctcttttttcccccccaacaaagtttttttgttttttgtttttttttttaagtagaaattccctccagttttattagcctctttatatgtctcaaattgcatgaattttttctggctgttggaaacctgaatgcttttagacccaaatggaaaatttctgaaatgctggattatctatttttaaacaagcagttgacttaaaactttctgtggcaacttctggttttctgacagttcccagtgagagaaatgctgaaagtacactgggatcactgggacactgtcttatgaaggtttgcttgggatgaaaaaggatattgcagcttcagcagtgttgaactgtgtgtttaaaaatgtgaattactgttattgtatactgtaattgattacatgggctgggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttggaaaaaaaaactggtgttatgaagaacgtaaatgcactgtttttatttttattttatataatttaaattgactttcccactgtctttaagttgaaactgttaagctgaataaaaacttaagctgcaaattgataacttcgctacataacaaggaaaatataaatgtttacaaacagcttaaagatttgcatgtgcagtgtgcatttataacaaacttctaattgcacaaaacccatgccagctcagagtttaggtgtacacatttacccagttgagcgttcttagaataactactgcacaagttgacaataggtcgttctctctttttttttgtttgctccctttttctttttctccccttcctccttaccctccctcccttactctccccccccaccaccaccctccacccccaactcatgaaaagattctatggactgaaaaagccccaggctgaaaggactggactgccttgattgacatggggaagggggttagtagactatgtggattgcggcagcagaggctgcagcctaacgtgtggttttaatgaccagcacgcaaggcaaaagcattttgcacagtgtttgttttcctgtcttgcacttacaaataaggtctatgggagtagcatggaaaacgtttgctgtttttcccttttttttttaattgcttttgtttaaaatttgatcgccttaactactgtaaacatagcctatttttgtgcttaagatactgaatggaaaactccattgtgtgttgctggactgttttggaaatatttggttaaatgtgtgttaatttggctgtaatggcatttaaagcaaacaaacaaacaaaaaaagctgtgaaaatggccttggagcattatctttagttacttgaagagtttctagtttttttaaaatacagtttatgttaaaataatttttattaatttagagaagacaatcaatgtctgtgagaaaacggactttcttttggattttctttttgtggtcattgtgagtgattgctttttccttttcttagtttcacattcttcctttgttctaaaacttagactgacatctagctttgacaatcatagtatgttttattttcctgagggggaataacttataatgctgtttagttttgtactattggtgtgttggtgaatttttaaactgtgtgctaactgcaataaattatatgaactgagaaaaaaaaaaaaaaaaaaaaaaaaaaaaaaaaaaaaaaaaaaaaaaaaa

SEQ ID NO: 83 - DNA, 1569 bp, Homo sapiens
atggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataa

SEQ ID NO: 84 - protein, 522 aa, Homo sapiens
MEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHIAGDLPSDEDEGEDEKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMESSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 85 - DNA, 2450 bp, Homo sapiens
agaacggcttccggcgggagctgtgcagctccttatcatggggacaattcatctctttcgaaaaccacaaagatccttttttggcaagttgttacgggaatttagacttgtagcagctgaccgaaggtcctggaagatactgctctttggtgtaataaacttgatatgtactggcttcctgcttatgtggtgcagttctactaatagtatagctttaactgcctatacttacctgaccatttttgatctttttagtttaatgacatgtttaataagttactgggtaacattgaggaaacctagccctgtctattcatttgggtttgaaagattagaagtcctggctgtatttgcctccacagtcttggcacagttgggagctctctttatattaaaagaaagtgcagaacgctttttggaacagcccgagatacacacgggaagattattagttggtacttttgtggctctttgtttcaacctgttcacgatgctttctattcggaataaaccttttgcttatgtctcagaagctgctagtacgagctggcttcaagagcatgttgcagatcttagtcgaagcttgtgtggaattattccgggacttagcagtatcttccttccccgaatgaatccatttgttttgattgatcttgctggagcatttgctctttgtattacatatatgctcattgaaattaataattattttgccgtagacactgcctctgctatagctattgccttgatgacatttggcactatgtatcccatgagtgtgtacagtgggaaagtcttactccagacaacaccaccccatgttattggtcagttggacaaactcatcagagaggtatctaccttagatggagttttagaagtccgaaatgaacatttttggaccctaggttttggctcattggctggatcagtgcatgtaagaattcgacgagatgccaatgaacaaatggttcttgctcatgtgaccaacaggctgtacactctagtgtctactctaactgttcaaattttcaaggatgactggattaggcctgccttattgtctgggcctgttgcagccaatgtcctaaacttttcagatcatcacgtaatcccaatgcctcttttaaagggtactgatgatttgaacccagttacatcaactccagctaaacctagtagtccacctccagaattttcatttaacactcctgggaaaaatgtgaacccagttattcttctaaacacacaaacaaggccttatggttttggtctcaatcatggacacacaccttacagcagcatgcttaatcaaggacttggagttccaggaattggagcaactcaaggattgaggactggttttacaaatataccaagtagatatggaactaataatagaattggacaaccaagaccatgatagactctaacttatttttataaggaatattgactccttggcttccaatttatttagtaatccaactttgcattgactgtttaatcatttactctaaatgttagataatagtagtcttgttcacatttcatgaaacctatgaaactatatttttgtaaaatgtatttgtgacagtgaaatcctcgtaaatgttaaaggctttaaataggcttcctttagaaaatgtgtttctttaaatttggattttggtatctttggttttgtagttgactgcagtgtgatgtgaccttacctttataagagccacttgatggagtagatctgtcacattactaagatacgatatttctttttttttccgagacggagtcttgctctgccactgtgcccggccaatacattattattaacttaaggctgtactttattaaggcttccttagtttttgttttgttttgttttttgagatggagtctcactctgtcgcccaggctggaatgcagtggcatgatctcagctcactgcaacctctgcctcctgagttcaaatgattctcctgcctcagcctcccgagtagctgggattacaggcacctgccaccacgcccagctaatttttgtatttttagtaaagacgggggatttcaccatgttggccaggctggtcttgaactcctgacctcatgatccacccaccttagcctcccaaagtgctgggattaggtgtgagccaccgcacctggccgatattttctttaatgaaatttataaatatgcttcttgaataatacacattttgggaaagggaaaaatgtctgttcaaaaagtaaaggtctcttttatagcttttccaaacttaattgctaaatttttctttgaggttctcctgaattatgtcttacaaactaaaagcaaaaatttttagcagaaattttggaatacattctatctagcacaatttgaatttttaattatcaagatttttgttaaagtttctctcctttaaaaattttagtacatttgtaaat

SEQ ID NO: 86 - DNA, 1386 bp, Homo sapiens
atggggacaattcatctctttcgaaaaccacaaagatccttttttggcaagttgttacgggaatttagacttgtagcagctgaccgaaggtcctggaagatactgctctttggtgtaataaacttgatatgtactggcttcctgcttatgtggtgcagttctactaatagtatagctttaactgcctatacttacctgaccatttttgatctttttagtttaatgacatgtttaataagttactgggtaacattgaggaaacctagccctgtctattcatttgggtttgaaagattagaagtcctggctgtatttgcctccacagtcttggcacagttgggagctctctttatattaaaagaaagtgcagaacgctttttggaacagcccgagatacacacgggaagattattagttggtacttttgtggctctttgtttcaacctgttcacgatgctttctattcggaataaaccttttgcttatgtctcagaagctgctagtacgagctggcttcaagagcatgttgcagatcttagtcgaagcttgtgtggaattattccgggacttagcagtatcttccttccccgaatgaatccatttgttttgattgatcttgctggagcatttgctctttgtattacatatatgctcattgaaattaataattattttgccgtagacactgcctctgctatagctattgccttgatgacatttggcactatgtatcccatgagtgtgtacagtgggaaagtcttactccagacaacaccaccccatgttattggtcagttggacaaactcatcagagaggtatctaccttagatggagttttagaagtccgaaatgaacatttttggaccctaggttttggctcattggctggatcagtgcatgtaagaattcgacgagatgccaatgaacaaatggttcttgctcatgtgaccaacaggctgtacactctagtgtctactctaactgttcaaattttcaaggatgactggattaggcctgccttattgtctgggcctgttgcagccaatgtcctaaacttttcagatcatcacgtaatcccaatgcctcttttaaagggtactgatgatttgaacccagttacatcaactccagctaaacctagtagtccacctccagaattttcatttaacactcctgggaaaaatgtgaacccagttattcttctaaacacacaaacaaggccttatggttttggtctcaatcatggacacacaccttacagcagcatgcttaatcaaggacttggagttccaggaattggagcaactcaaggattgaggactggttttacaaatataccaagtagatatggaactaataatagaattggacaaccaagaccatga

SEQ ID NO: 87 - protein, 461 aa, Homo sapiens
MGTIHLFRKPQRSFFGKLLREFRLVAADRRSWKILLFGVINLICTGFLLMWCSSTNSIALTAYTYLTIFDLFSLMTCLISYWVTLRKPSPVYSFGFERLEVLAVFASTVLAQLGALFILKESAERFLEQPEIHTGRLLVGTFVALCFNLFTMLSIRNKPFAYVSEAASTSWLQEHVADLSRSLCGIIPGLSSIFLPRMNPFVLIDLAGAFALCITYMLIEINNYFAVDTASAIAIALMTFGTMYPMSVYSGKVLLQTTPPHVIGQLDKLIREVSTLDGVLEVRNEHFWTLGFGSLAGSVHVRIRRDANEQMVLAHVTNRLYTLVSTLTVQIFKDDWIRPALLSGPVAANVLNFSDHHVIPMPLLKGTDDLNPVTSTPAKPSSPPPEFSFNTPGKNVNPVILLNTQTRPYGFGLNHGHTPYSSMLNQGLGVPGIGATQGLRTGFTNIPSRYGTNNRIGQPRP

SEQ ID NO: 88 - DNA, 5052 bp, Homo sapiens
cggcgcttttccttcggactaagggagccgtcgaagagcgctcgccaaaggccagccgtttctccctacggtgccgccgctcctcctgcagccgcccgtaggtagcgggccgttttcctcacctgtccctgacaggcgccctcagggagccgcggtccgcgatgtcaagcgaggaaagctaccgggccatcctgcgttacctgacgaacgagcgcgagccgtatgcgccgggcaccgagggcaatgtcaagcgtaaaatccgaaaagctgccgcctgctacgtggtgcgcggcgggactctgtattaccagcggcggcagcggcaccgcaagaccttcgcggagctggaggtggtgctgcagccggagcgacgccgggacctcatcgaggcggcgcacctgggtcccggcggcactcaccacacccggcatcagacctggcactacttgtccaagacgtactggtggcgaggtatattgaagcaagtcaaagattacattaaacagtgtagcaaatgccaggagaaactagatcgatcccgtccaatatcagatgtttcagaaatgttggaagaattgggactagaccttgaatctggagaagaaagtaatgaatcggaagatgacctgagcaactttacttcatctccaactacagcatccaagcctgcaaaaaagaagccagtatccaaacatgaacttgtgtttgttgacaccaaaggagtggtaaaacgttcttctccaaaacattgtcaggctgtcttaaaacagctgaacgaacagagactttccaaccagttctgtgatgttactttgttaattgaaggagaagagtacaaagctcataaatctgttttgtcagcaaatagcgagtattttcgagatctttttattgagaaaggagctgtttccagtcatgaggctgtggtggatctttctggtttttgtaaggccagcttccttcctttactggaatttgcctatacttctgtactaagttttgatttctgtagcatggctgatgtagccatcttagctcgtcatcttttcatgtcagaagtcttagagatttgtgaaagtgtacataagctaatggaagagaagcagctaacagtatataagaagggcgaagtacaaacagttgcatccacccaggacttacgagtacagaatggaggtacagcacctcctgttgctagcagtgagggaaccacaacaagtttacctactgaacttggggattgtgaaattgtactactggtaaatggagaattgccagaagctgagcagaatggagaggtaggacgacagcctgagccccaggtttcttcagaggctgaatctgccctgtcatcagtaggatgtatagctgattcccatcctgaaatggagtctgttgatttaataacaaaaaacaaccagacagaactagaaacttcaaacaacagagaaaataacacagtttctaatatacaccctaaactttcaaaagagaatgtaattagtagctcgccagaggatagtggtatgggaaatgatatatcagctgaggatatttgtgccgaagacattccaaaacataggcagaaagttgaccaacctttaaaagatcaggaaaatctagttgcatcaacagcaaagacaaactttggccctgatgatgatacttatagaagcaggcttcgacaacgttctgttaatgaaggggcatatattcgactacacaagggaatggagaaaaagctgcagaaacggaaagccgttcccaagtcagcagttcaacaggtggctcagaagttagttcaaagaggaaaaaagatgaaacagccaaaaagagatgctaaagagaacacagaagaagcatctcataaatgtggggaatgtggaatggtttttcagagacgatacgcccttataatgcacaaactgaaacatgaaagagctagagattacaaatgtccattgtgtaaaaaacagtttcagtacagtgcctctttgcgagcacatcttattcgtcataccagaaaagatgcaccctcttcatcctcgtccaattccacgtctaatgaagcatcgggaacatcatctgagaagggcagaaccaagcgggaatttatatgttccatatgtggaagaacattacctaaattatattctctccgaatacatatgttaaagcacacaggtgtaaagccacatgcatgccaggtctgtggaaagacttttatctataagcatggtctaaaattacatcagagtcttcatcaatcacagaagcagttccagtgtgaactgtgtgttaagtcatttgttaccaaacggagtcttcaagaacatatgagtattcacacaggagagtccaagtacctttgctcagtttgtggaaagtcttttcataggggctctggactcagcaagcacttcaagaaacaccaaccaaagcctgaggttcgaggctatcattgtactcaatgtgaaaaaagtttctttgaagctagagatcttcgccagcacatgaacaaacatcttggtgtgaagccattccagtgccaattttgtgataagtgctatagttggaagaaagattggtattcccatgtgaagtctcattctgtcactgagccttataggtgtaatatatgtggcaaagaattttatgaaaaagctttgttcagaaggcatgtaaagaaagctacccatgggaagaaaggaagagcaaagcaaaacctggaacgggtgtgtgaaaaatgtggaagaaaattcactcagctaagagagtataggagacacatgaacaaccatgaaggagttaagccatttgagtgcttaacatgtggagtagcttgggctgatgcccgatctctaaaacgccatgtcagaacacatactggtgaacggccctatgtctgtcctgtatgtagcgaagcctacatagatgctcgaacactccgtaaacatatgactaaattccacagagactatgtgccttgcaaaattatgctggaaaaagacacccttcagtttcataaccaaggaactcaagtggcacatgctgttagcatcttaacagcaggcatgcaggaacaagaaagcagtggtcctcaagaacttgagactgtggtagtgacaggagaaactatggaagctctggaagctgttgcagctactgaagagtatccatcggtatctacactttctgaccaaagtattatgcaagtggttaattatgtattagcacaacagcaaggacagaagctatctgaagttgcagaagctattcaaactgttaaagtagaggtagcacatatttcaggaggagaatgagtatgttaatgaagataaaaagaagtgacatctcttgtacactgaactcacagaacatttgtttacaattctgtgtgactgtctgcttggagtttacatatcaaagttctgggctgtttggtaacgtaacgtttccaaacattttgtctggccaatgggttctatagaaaagaccgtttagtgtagagaaattgaaaacagatctattaggttggtgcaattgcttttgcaccaacctaatatttgatggcagtggtttatcatgatataccttttatgaattaatgtttataaatgactgtactgaatttaaaaccgtacagtttcatttgcattttgacattactttattatacattttgcatttaaaaggctgcaccagttggcttttcttctgttttattctcaaaatatagagattctgtgatttatttgccctgtttatggattaaaaagaaaattctaatataaagcatttcaataggatgcataggtatattacgttttttaaatgctttagatctgtgattcttgacttactatttattttatccccttttaagtcagggatgctttattctattttaaagcacttatgagttacatgttgtaatcaagtttgcacaatatatttatctatatgaggaacccataaatgaatagctaatttttaaaatgccattaaaatgcatgaaatgcttattaaaaccttactatactatttcttcaagggcaagtaaattgaccatgagaaaagaacacagttattaaacactgttgacaggaaaattctccttgataacataggacaattaatggaaaaaaaaattctcattatttgcaaagaatgaacaagttaatgaacaaacaaactagatttggtatgttttcagcttttgtatcatgtttaattgtttaatttggttgaaaaactgcagttgagaaatcagatagcaatatagacattcacagcagctctgtggataccatgtaattgtcaggtaatttcagaatgttgaaaattattcagtgcagccctcatagtatcatacttgaagaaattgattaccgttccactaaattgttgaagataaattatttttaaaggttatgaaaactaagttatattaattcatatgtttgatttttaaatcccacctcctcaagctatccaattttctgactttgaaaataaccatgagagatgccacatttctctctgggaaactaccactcaaagaataattgttaaaaattaagcttttaggtattagaagctgttataaagtataaaattaagatataagcagatcacatgtaaatcattcctaaagcacaagaaaagaatgtgccttgatgtacatatattactaagttgcctctcccagtttactttaaaaatggctttaaggataaagaataaatgtgatagctgtgcatgcattatatatttgcatttgcaaatttcccattgttttaacagctgtgtggctgactttcaattttaagacgtgaattgacatacagcccataactttataatggctgctcatttatcttatctttcagttagtggaaaaacatttcaacctgactaaaatttggaattgtgtctttttatgttccatcctctgttgttactagatttagtttaaaaattgtgtatgaccattaatgtatgtcataaacatgtaaataaaagatgttgaatcttgttgaaagcgcggcc

SEQ ID NO: 89 - DNA, 3162 bp, Homo sapiens
atgtcaagcgaggaaagctaccgggccatcctgcgttacctgacgaacgagcgcgagccgtatgcgccgggcaccgagggcaatgtcaagcgtaaaatccgaaaagctgccgcctgctacgtggtgcgcggcgggactctgtattaccagcggcggcagcggcaccgcaagaccttcgcggagctggaggtggtgctgcagccggagcgacgccgggacctcatcgaggcggcgcacctgggtcccggcggcactcaccacacccggcatcagacctggcactacttgtccaagacgtactggtggcgaggtatattgaagcaagtcaaagattacattaaacagtgtagcaaatgccaggagaaactagatcgatcccgtccaatatcagatgtttcagaaatgttggaagaattgggactagaccttgaatctggagaagaaagtaatgaatcggaagatgacctgagcaactttacttcatctccaactacagcatccaagcctgcaaaaaagaagccagtatccaaacatgaacttgtgtttgttgacaccaaaggagtggtaaaacgttcttctccaaaacattgtcaggctgtcttaaaacagctgaacgaacagagactttccaaccagttctgtgatgttactttgttaattgaaggagaagagtacaaagctcataaatctgttttgtcagcaaatagcgagtattttcgagatctttttattgagaaaggagctgtttccagtcatgaggctgtggtggatctttctggtttttgtaaggccagcttccttcctttactggaatttgcctatacttctgtactaagttttgatttctgtagcatggctgatgtagccatcttagctcgtcatcttttcatgtcagaagtcttagagatttgtgaaagtgtacataagctaatggaagagaagcagctaacagtatataagaagggcgaagtacaaacagttgcatccacccaggacttacgagtacagaatggaggtacagcacctcctgttgctagcagtgagggaaccacaacaagtttacctactgaacttggggattgtgaaattgtactactggtaaatggagaattgccagaagctgagcagaatggagaggtaggacgacagcctgagccccaggtttcttcagaggctgaatctgccctgtcatcagtaggatgtatagctgattcccatcctgaaatggagtctgttgatttaataacaaaaaacaaccagacagaactagaaacttcaaacaacagagaaaataacacagtttctaatatacaccctaaactttcaaaagagaatgtaattagtagctcgccagaggatagtggtatgggaaatgatatatcagctgaggatatttgtgccgaagacattccaaaacataggcagaaagttgaccaacctttaaaagatcaggaaaatctagttgcatcaacagcaaagacaaactttggccctgatgatgatacttatagaagcaggcttcgacaacgttctgttaatgaaggggcatatattcgactacacaagggaatggagaaaaagctgcagaaacggaaagccgttcccaagtcagcagttcaacaggtggctcagaagttagttcaaagaggaaaaaagatgaaacagccaaaaagagatgctaaagagaacacagaagaagcatctcataaatgtggggaatgtggaatggtttttcagagacgatacgcccttataatgcacaaactgaaacatgaaagagctagagattacaaatgtccattgtgtaaaaaacagtttcagtacagtgcctctttgcgagcacatcttattcgtcataccagaaaagatgcaccctcttcatcctcgtccaattccacgtctaatgaagcatcgggaacatcatctgagaagggcagaaccaagcgggaatttatatgttccatatgtggaagaacattacctaaattatattctctccgaatacatatgttaaagcacacaggtgtaaagccacatgcatgccaggtctgtggaaagacttttatctataagcatggtctaaaattacatcagagtcttcatcaatcacagaagcagttccagtgtgaactgtgtgttaagtcatttgttaccaaacggagtcttcaagaacatatgagtattcacacaggagagtccaagtacctttgctcagtttgtggaaagtcttttcataggggctctggactcagcaagcacttcaagaaacaccaaccaaagcctgaggttcgaggctatcattgtactcaatgtgaaaaaagtttctttgaagctagagatcttcgccagcacatgaacaaacatcttggtgtgaagccattccagtgccaattttgtgataagtgctatagttggaagaaagattggtattcccatgtgaagtctcattctgtcactgagccttataggtgtaatatatgtggcaaagaattttatgaaaaagctttgttcagaaggcatgtaaagaaagctacccatgggaagaaaggaagagcaaagcaaaacctggaacgggtgtgtgaaaaatgtggaagaaaattcactcagctaagagagtataggagacacatgaacaaccatgaaggagttaagccatttgagtgcttaacatgtggagtagcttgggctgatgcccgatctctaaaacgccatgtcagaacacatactggtgaacggccctatgtctgtcctgtatgtagcgaagcctacatagatgctcgaacactccgtaaacatatgactaaattccacagagactatgtgccttgcaaaattatgctggaaaaagacacccttcagtttcataaccaaggaactcaagtggcacatgctgttagcatcttaacagcaggcatgcaggaacaagaaagcagtggtcctcaagaacttgagactgtggtagtgacaggagaaactatggaagctctggaagctgttgcagctactgaagagtatccatcggtatctacactttctgaccaaagtattatgcaagtggttaattatgtattagcacaacagcaaggacagaagctatctgaagttgcagaagctattcaaactgttaaagtagaggtagcacatatttcaggaggagaatga

SEQ ID NO: 90 - protein, 1053 aa, Homo sapiens
MSSEESYRAILRYLTNEREPYAPGTEGNVKRKIRKAAACYVVRGGTLYYQRRQRHRKTFAELEVVLQPERRRDLIEAAHLGPGGTHHTRHQTWHYLSKTYWWRGILKQVKDYIKQCSKCQEKLDRSRPISDVSEMLEELGLDLESGEESNESEDDLSNFTSSPTTASKPAKKKPVSKHELVFVDTKGVVKRSSPKHCQAVLKQLNEQRLSNQFCDVTLLIEGEEYKAHKSVLSANSEYFRDLFIEKGAVSSHEAVVDLSGFCKASFLPLLEFAYTSVLSFDFCSMADVAILARHLFMSEVLEICESVHKLMEEKQLTVYKKGEVQTVASTQDLRVQNGGTAPPVASSEGTTTSLPTELGDCEIVLLVNGELPEAEQNGEVGRQPEPQVSSEAESALSSVGCIADSHPEMESVDLITKNNQTELETSNNRENNTVSNIHPKLSKENVISSSPEDSGMGNDISAEDICAEDIPKHRQKVDQPLKDQENLVASTAKTNFGPDDDTYRSRLRQRSVNEGAYIRLHKGMEKKLQKRKAVPKSAVQQVAQKLVQRGKKMKQPKRDAKENTEEASHKCGECGMVFQRRYALIMHKLKHERARDYKCPLCKKQFQYSASLRAHLIRHTRKDAPSSSSSNSTSNEASGTSSEKGRTKREFICSICGRTLPKLYSLRIHMLKHTGVKPHACQVCGKTFIYKHGLKLHQSLHQSQKQFQCELCVKSFVTKRSLQEHMSIHTGESKYLCSVCGKSFHRGSGLSKHFKKHQPKPEVRGYHCTQCEKSFFEARDLRQHMNKHLGVKPFQCQFCDKCYSWKKDWYSHVKSHSVTEPYRCNICGKEFYEKALFRRHVKKATHGKKGRAKQNLERVCEKCGRKFTQLREYRRHMNNHEGVKPFECLTCGVAWADARSLKRHVRTHTGERPYVCPVCSEAYIDARTLRKHMTKFHRDYVPCKIMLEKDTLQFHNQGTQVAHAVSILTAGMQEQESSGPQELETVVVTGETMEALEAVAATEEYPSVSTLSDQSIMQVVNYVLAQQQGQKLSEVAEAIQTVKVEVAHISGGE

SEQ ID NO: 91 - DNA, 4262 bp, Homo sapiens
ctcctaactccactaagtacagaagtggagccgtacagccatgaatgtcaggcatgttgtggggactagttgtattagttttctattgccaccataacaaattaccacaaacttaagcaatatagtacaaatttatcttaaagttattggcaggtcagaagcctgatacggatctcattgggctaaaacgaaggagttggcagggctgtgtgttcctttctgtaggctctgggcaggatccatttcctgcacttttttggcttttggaggtcgtccacattccttggctcctggccctgctccttcatcttcaaagctaccaaagatcggcttcagctccccaggaatatgattgaaaacagcatgtttgaggaagaaccagatgtggtggatttagccaaagagccttgtttacatcctctagagcctgatgaggtggaatatgagccccggggttcccgactgctggtgcggggtcttggtgagcatgagatggaggaggatgaagaggattatgagtcatcagcaaagctgctgggcatgtccttcatgaatagaagctcaggccttcggaacagtgcaactggttacaggcagagcccagatggggcttgttcagtaccctctgcaaggaccatggtggtctgtgcttttgtcatcgtggttgctgtttctgtaatcatggtgatttacttactgcccagatgtacctttaccaaagaaggctgccataaaaaaaaccagtcaattggactaattcagccatttgcaacaaatgggaaattgtttccatgggcacagatcaggcttcccactgccgttgtgccactacgctatgaactcagcctacacccgaacctaacctcgatgacattcaggggttctgtgacaatttcagttcaggctcttcaggtcacatggaatatcattcttcatagcacaggtcataatatttcaagagtgacctttatgtcagcagtttcaagccaagaaaaacaagctgagatcctggaatatgcatatcatggacagatcgccattgttgcccccgaagcccttctagcagggcacaattatacgttgaagatagagtactcggcaaatatatctagttcttattatgggttttatggcttctcctacacagatgaaagtaatgagaaaaagtactttgcagcaactcagtttgaacccctggcagcaagatctgcttttccttgttttgatgaaccagcatttaaagccacttttatcatcaagatcataagggatgagcaatacaccgctttatcaaatatgcctaagaagtcatcagtcgttctagatgatggacttgttcaggatgagttttctgagagtgtgaagatgagcacttacttggttgctttcattgtgggagagatgaagaacctgagtcaggacgtaaatggaaccctggtttctatatatgctgtaccagaaaagattggtcaagttcattatgccttggaaacaactgtgaagcttcttgagttttttcaaaactactttgaaattcagtacccacttaagaaattggatttggtggctattcctgactttgaagcaggagcaatggaaaattggggtttgctcaccttccgagaggagacacttctgtatgacagtaacacttcttcaatggcggatagaaagctggtgactaaaatcattgctcatgagctggcccaccagtggtttggcaatctggtaacaatgaagtggtggaatgacctatggctaaatgaaggttttgccactttcatggagtatttctctttggaaaaaatattcaaagagctttctagttatgaagatttcttagatgctcgatttaaaaccatgaagaaagattccttaaattcatctcatccaatatcatcatctgttcagtcttcagaacaaattgaagaaatgtttgattctctttcctattttaagggatcttctctcttgttgatgttgaaaacttaccttagtgaagatgtgtttcaacatgctgttgtcctttacctgcataatcacagctatgcatctattcaaagtgatgatctgtgggatagttttaatgaggtcacaaaccaaacactagatgtaaagagaatgatgaaaacctggaccctgcagaaaggatttcctttagtgactgttcaaaagaaaggaaaggaactttttatacaacaagagagattctttttaaatatgaagcctgaaattcagccttcagatacaagctacctgtggcatattccactatcctatgtcactgaaggaagaaattattcaaaatatcaatcggtatcattactggataagaaatcaggtgtcatcaatcttacagaagaagtgctgtgggtcaaagtgaatataaacatgaatggttattatattgtacactatgcagatgatgattgggaagcactaatccatcagttgaaaataaatccttatgttctgagtgacaaagaccgagccaaccttatcaacaacatctttgaacttgcaggcctaggcaaggtacctctcaagagggcctttgatttgattaattatcttggaaatgagaaccatactgcacccatcaccgaagccctgtttcagacagacctcatctataacctccttgaaaaactgggatacatggatctggcctcaagactggtgactagggtatttaaattacttcaaaaccaaattcaacaacaaacttggactgatgagggcactccatctatgcgagagcttcggtcagccctgctagagtttgcttgcacccacaacctggggaactgctctactactgccatgaaactgtttgatgactggatggcatccaatggaactcaaagcctacctactgatgtcatgacaactgtgttcaaagttggagcaaaaactgacaaaggctggtcattccttttgggcaaatacatttctataggctctgaagcagagaagaacaaaatactagaagcacttgccagctcagaggatgtgcggaagctttactggttaatgaaaagtagcctgaatggagataacttccgaacacagaagctgtcttttatcattagaacagtgggtcgacattttcctggacacttactggcatgggattttgtcaaagagaactggaataagcttgtacagaagttccctctggggtcctataccatacaaaatattgttgctggatcaacttacctgttttcaacaaagacacatttatctgaggttcaggcattctttgaaaatcagtcagaggcaaccttccggcttcgttgtgtccaggaggctttggaagtcattcagttgaatatccagtggatggagaagaacctcaaaagtctcacatggtggctgtagcatgcacaaccgcacctcattttgttgcccattcagagagcttgtaagcttgggctctgccgcttttgcaaaagccaaggtaaagccaggatcgctgccaagttgtttgcactctttggagttctagttagctcagggcctgactgtatttttcatccatcttttctgaagtgtctttgggcagtatgtagttatttattacaaaattatattcacctaaatgccaaccatctacaaaaacaatgagtaatttttctactttgaagatacacagatggggacaaaaaccctgttttggaattctgttctattcctcagtatccagaaagttactgacacagtaaaacaaggaaagttctaccctaagagccgccatcacttcaggccgctggtttgtcagccatctgttgcttcttattgatagatggcattggaatgtggtacaaagttagctctgaagaatatggtaacgaagacaataaagcatgcactgtaagaactgacctcaggtgtgcagatctactttgatttggggttttgattaattctttatttttctgaaaaaaagtttaaataatgtctgtaactattgtgtttttttcctgccgaatagccaggtgctacagatttttaaaatttttgttgtattgaaaagctaaacaaggccaaaaggttaaattttttgaatatttaagactttctttttcatcttttatagcgttaccataggaaactgttccaaactgagttagctttaattataattattttattcataaatggtgatagtccccagatctgtacacctttatcactccctgccgtagatatactttaggttagtatttctacattcgtggcaagcattttggtaacac

SEQ ID NO: 92 - DNA, 3036 bp, Homo sapiens
atgattgaaaacagcatgtttgaggaagaaccagatgtggtggatttagccaaagagccttgtttacatcctctagagcctgatgaggtggaatatgagccccggggttcccgactgctggtgcggggtcttggtgagcatgagatggaggaggatgaagaggattatgagtcatcagcaaagctgctgggcatgtccttcatgaatagaagctcaggccttcggaacagtgcaactggttacaggcagagcccagatggggcttgttcagtaccctctgcaaggaccatggtggtctgtgcttttgtcatcgtggttgctgtttctgtaatcatggtgatttacttactgcccagatgtacctttaccaaagaaggctgccataaaaaaaaccagtcaattggactaattcagccatttgcaacaaatgggaaattgtttccatgggcacagatcaggcttcccactgccgttgtgccactacgctatgaactcagcctacacccgaacctaacctcgatgacattcaggggttctgtgacaatttcagttcaggctcttcaggtcacatggaatatcattcttcatagcacaggtcataatatttcaagagtgacctttatgtcagcagtttcaagccaagaaaaacaagctgagatcctggaatatgcatatcatggacagatcgccattgttgcccccgaagcccttctagcagggcacaattatacgttgaagatagagtactcggcaaatatatctagttcttattatgggttttatggcttctcctacacagatgaaagtaatgagaaaaagtactttgcagcaactcagtttgaacccctggcagcaagatctgcttttccttgttttgatgaaccagcatttaaagccacttttatcatcaagatcataagggatgagcaatacaccgctttatcaaatatgcctaagaagtcatcagtcgttctagatgatggacttgttcaggatgagttttctgagagtgtgaagatgagcacttacttggttgctttcattgtgggagagatgaagaacctgagtcaggacgtaaatggaaccctggtttctatatatgctgtaccagaaaagattggtcaagttcattatgccttggaaacaactgtgaagcttcttgagttttttcaaaactactttgaaattcagtacccacttaagaaattggatttggtggctattcctgactttgaagcaggagcaatggaaaattggggtttgctcaccttccgagaggagacacttctgtatgacagtaacacttcttcaatggcggatagaaagctggtgactaaaatcattgctcatgagctggcccaccagtggtttggcaatctggtaacaatgaagtggtggaatgacctatggctaaatgaaggttttgccactttcatggagtatttctctttggaaaaaatattcaaagagctttctagttatgaagatttcttagatgctcgatttaaaaccatgaagaaagattccttaaattcatctcatccaatatcatcatctgttcagtcttcagaacaaattgaagaaatgtttgattctctttcctattttaagggatcttctctcttgttgatgttgaaaacttaccttagtgaagatgtgtttcaacatgctgttgtcctttacctgcataatcacagctatgcatctattcaaagtgatgatctgtgggatagttttaatgaggtcacaaaccaaacactagatgtaaagagaatgatgaaaacctggaccctgcagaaaggatttcctttagtgactgttcaaaagaaaggaaaggaactttttatacaacaagagagattctttttaaatatgaagcctgaaattcagccttcagatacaagctacctgtggcatattccactatcctatgtcactgaaggaagaaattattcaaaatatcaatcggtatcattactggataagaaatcaggtgtcatcaatcttacagaagaagtgctgtgggtcaaagtgaatataaacatgaatggttattatattgtacactatgcagatgatgattgggaagcactaatccatcagttgaaaataaatccttatgttctgagtgacaaagaccgagccaaccttatcaacaacatctttgaacttgcaggcctaggcaaggtacctctcaagagggcctttgatttgattaattatcttggaaatgagaaccatactgcacccatcaccgaagccctgtttcagacagacctcatctataacctccttgaaaaactgggatacatggatctggcctcaagactggtgactagggtatttaaattacttcaaaaccaaattcaacaacaaacttggactgatgagggcactccatctatgcgagagcttcggtcagccctgctagagtttgcttgcacccacaacctggggaactgctctactactgccatgaaactgtttgatgactggatggcatccaatggaactcaaagcctacctactgatgtcatgacaactgtgttcaaagttggagcaaaaactgacaaaggctggtcattccttttgggcaaatacatttctataggctctgaagcagagaagaacaaaatactagaagcacttgccagctcagaggatgtgcggaagctttactggttaatgaaaagtagcctgaatggagataacttccgaacacagaagctgtcttttatcattagaacagtgggtcgacattttcctggacacttactggcatgggattttgtcaaagagaactggaataagcttgtacagaagttccctctggggtcctataccatacaaaatattgttgctggatcaacttacctgttttcaacaaagacacatttatctgaggttcaggcattctttgaaaatcagtcagaggcaaccttccggcttcgttgtgtccaggaggctttggaagtcattcagttgaatatccagtggatggagaagaacctcaaaagtctcacatggtggctgtag

SEQ ID NO: 93 - protein, 1011 aa, Homo sapiens
MIENSMFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 94 - DNA, 6861 bp, Homo sapiens
gggagagggaagggggcggagctagccggagccgcgagtccattttggggctgtgcttggcgcgtaccgtgcggtccctgtagttggaggacgggcggtcgcgcggcctttcccactagccggaggtcggagataagtacccgccgcccggctcctctcgggaaagcggggtggtcctcgaaccttcagcgagggtggggagttgcccagtagcctctagttcgttagtcaaaacgtgaaaaaaaagacctgctttgccctgggaaatagtaaccctgccaaatacatcagcttgtaggagacagaggatgtgatggagctgcttgaagaagatctcacatgccctatttgttgtagtctgtttgatgatccacgggttttgccttgctcccacaacttctgcaaaaaatgcttagaaggtatcttagaagggagtgtgcggaattccttgtggagaccagctccattcaagtgtcctacatgccgtaaggaaacttcagctactggaattaatagcctgcaggttaattactccctgaagggtattgtggaaaagtataacaagatcaagatctctcccaaaatgccagtatgcaaaggacacttggggcagcctctcaacattttctgcctgactgatatgcagctgatttgtgggatctgtgctactcgtggggagcacaccaaacatgtcttctgttctattgaagatgcctatgctcaggaaagggatgcctttgagtccctcttccagagctttgagacctggcgtcggggagatgctctttctcgcttggataccttggaaactagtaagaggaaatccctacagttactgactaaagattcagataaagtgaaggaattttttgagaagttacaacacacactggatcaaaagaagaatgaaattctgtctgactttgagaccatgaaacttgctgttatgcaagcatatgacccagagatcaacaaactcaacaccatcttgcaggagcaacggatggcctttaacattgctgaggctttcaaagatgtgtcagaacccattgtatttctgcaacagatgcaggagtttagagagaaaatcaaagtaatcaaggaaactcctttacctccctctaatttgcctgcaagccctttaatgaagaactttgataccagtcagtgggaagacataaaactagtcgatgtggataaactttctttgcctcaagacactggcacattcattagcaagattccctggagcttttataagttatttttgctaatccttctgcttggccttgtcattgtctttggtcctaccatgttcctagaatggtcattatttgatgacctggcaacttggaaaggctgtctttcaaacttcagttcctatctgactaaaacagccgatttcatagaacaatcagttttttactgggaacaggtgacagatgggtttttcattttcaatgaaagattcaagaattttactttggtggtactgaacaatgtggcagaatttgtgtgcaaatataaactattataaaatctgtttcaagtatgcagttttcttttgttagaaattgttagagaatagagagtggtaattcagatttggtcaacgattctagtcacatattttcctccaaaagtattccttccaaaaataatctatacatgttcaaattaggtagcataaagataaaagtgaaatttagtagtataggcctgaacctttttttgtttaaaagagtgcttttgaaataagcatccaccccaaatgttggttgtatttatgctgtgataaaaataggtgagagatcatatgatctaatattgtattgatggaagtataggtagtatagtagtgattgttcttcaagcatgcagtaaagatcacttttttttttttttttttttttgagatggagtcttgctctgtcgcccaggctggagtgcagtgatgcaatcttggctcactgcagcctctacctcccaggttcaagtgattatcctgccttagcctcctgagtagctgggattacaggcgcgtgccaccacgcccagctaattttttgtatttttagttgagacagggtttcactgtcccttaagaccatcctgttagccaagatggtcttgatctcctgacctcgtgatccgcctgcctcggcctcccaaagtgctgggattacaggtgtgagccaccgtgcccagccaaagactgctcttaaagcacctttttgacagtgaacatggtctaaaaaagggaagatattgtagaagatttcacacacacacgcgtgtgtgggagacaactaaaggtattgaaggtactaattaattagatttccaaaattttctacagagagttaattatcaccaaaatgtgaatggtacatacaaaacctggcattttcttgtgataagtttacatttttaggagagtggagctttcaatctgcccttttctcctttgtttttgtagttttcaccaaagatgatcaactagaaatgttaagtggctatgcaagcaaaagcatagataggttaaaaaaaaggatcagctggctgggcagggtggctcatgtctataatctcagcattctaggacagtgatgtgggaggattatttgaggctaggagttccagttcgaaaccagcttcgtaaacatagcaagacccagtctctaccaaaaaaaaaaaaaaaaaaaaaaaaaaaaaaaatatatatatatatatacacacacacacacacatatgtacacatacatatatatacatatatatgtatatatagaacacatggcaagagcaggaaatgggaagactaccttttttctggcagctatgtgttgttttgttcgaaagatggcaagtgtacaaccaaaccaatccacttgcttacagaaagaatgtttagtgtagttaagactaatttgacaacagataaaacagtctgtcagctattacaaagtaatcagctgaacactaatgctgttctgtctgctcacaagagataaagataccctctcatggaataaacaccttgcttagaattcatataacaaagaagtaaccttataactgctctctagtctgccttatttgtgtcatagagtaagatattccttgaaagcccattaagtggaatagactttctgaaaataagagttctttacctcagtcctcctctaatttggttgagaggaggtaatgaaaatggggacaggataaaaggtggcaactaaatttaaagcacagaagaaatgatttccttctagctatgagaatagtcaaattgagcttgccaggctgtctcattcctaacatgtaattcatgatttagccactaaatttctaaggagatcataagaaattaagaaaaacgtttaagtctagcctctgcgtggtagacaggtatggggagggagaatgcttttcccctcccagtaataaaaaaaaaaaaaaaaaaaatatatatatatatatatatatatatatatatatatatatatatatatatatatatagttttactaggttttcatggataagtttttaaatgtaagacaggaaagggatctatttgatgtctatcttcagatatattggcagttttccttaagctatttagttcctcatctgttgctttttcattttgtatactgcaagttcccaggcaactcgaatttgcaaacacagccatggatacactatttaccttacagtagtttcctgggaatctaagtctggtttttgttattcttccctcccctccactgcataatcatgtataactagcaacatttatggttataggttgatttcctaagtgtggctgatggtagcctctagtttgaagtgagggaagaatgagtagtcaggaactggtcactttgaatgtgggagggaagatattcacgacaaggttttctacgataaagcagtttcctgcttctcgtttggcacgcatgttagatggcagagaccaagaattcaagatggttggtggccagatttttgtagacagagatggtgatttgtttagtttcatcttagattttttgagaactcaccagcttttattacccactgaattttcagactatcttaggcttcagagagaggctcttttctatgaacttcgttctctagttgatctcttaaacccatacctgctacagccaagacctgctcttgtggaggtacatttcctaagccggaacactcaagcttttttcagggtgtttggctcttgcagcaaaacaattgagatgctaacagggaggattacagtgtttacagaacaaccttcagcgccgacctggaatggtaactttttccctcctcagatgaccttacttccactgcctccacaaagaccttcttaccatgacctggttttccagtgtggttctgacagcactactgataaccaaactggagtcaggtattttgtactttgcagtatttctcttgtataccagtttgtgatgttttctctaaaaacttgaagttcctcaggcctgtaacttctggaaaagatgattattcaaaataatgttttggggtaaccagtggagttgggtagaatgaccaaataattattttccaaactgggatactttttagagtgaaaggggctattattaggtgggacaaaaggaataaatgaagactgcccagaaaaaactgagactatggacattcaaatcatgggagaaaataattttgtagattatgttccattgctaatgaatttgacttagaaaagaattgccttatttttaagagattgtttcagtggttcacataaaggctcgctcactggtttctcttgagttccttacacactatataagttgttctttcagttttatgattcaactactgtttttccttcagctgactttatttttaaacacccttaaagacagatatatctcatggcaaatttggtatcctgttacagccttggctcttaaacaactcaaaatattgggataggctgtcagtatgttaaggatagttgctcctgagtcaattcttcacttactccctctgttgttcttggctggatcctaacgctgatttccactctgctgtcacaaacatttttccccccgtaaaatgtcttaatgctgtcctaccattattttaccaactgtgaaagctggctttaatttttaggaggaaaagaaaagcctgcatgtgttctttattggtatcatttaaaatatacttttttttttttttttggtaaaggtaggcgtattttaagatattttcttaacttgagcagtagccaacaggaaggataccagtgtctctctctcttagcgacacactccttggtcttgcttaccaactggaggacactaggtagaataaccgagtatgacaattcttaattgtttacattttataacttcctgtccttcaaaagagtttgaaatgtcattttgggaaaagagagccagtcaagctagtaggctgattgtgaagaaaatctaataccttatctttatctcaaacctctgtacaactttattttcattgatgggatactttaacaaaaatgaaattttttttggtttttaaaatatgagtgattatgacctctttggggatcatgcttcaaaaagtcagaaacctagagacaaaactgtcattgatttttaagaagaaacacactaggtcaaaagaagatgtcctggaaatatgaagtactctttaaaaaccatgcatttggagaaagtaattgtttccttgaaaaacatgattaaaaactaaaactgggatgttcctgtgtgtacacagtgccaaatggttttccctttttatgttgtgttttagaaacagcacgaaagttttttccattttaaagtgagaaaacattatatttagacttccataattccaaaatcagaagctatttttaaaattagcattttcttgcatcaccaaatggtattcaattgtttgaagctcaaaatttttaccattccataaatgtttgtgaatttttagacagtgccaatttaaaagtagagatagccaatctgaatacggtgaaattatggggatctctggtgattgggatgaaaactctggccttaaaaggtccacttttagtatataattgcctaattagcaatcatttttattttttgctcactccctggtctgaatctatctgtctattcagatattttttggtaggtttggaaaatggagaagtgagcctaattggtgcctaattgtctggtgtatcattcactttattcagtttgttctatcaatatgatttacccctcaaggttaacctagcaggttgctcagttattatctctcaaggtcacagtactagaaatacttggcttgcatctttcagatgccattcatgttatcaagctcaaattatagttggtcacaggattctaaagtctttatttgacttctcctttttgaactggctcaaatggaaaagtgtagttgcttttaaatgttaaaaataagtttaaactttatatttcccattggtttcccctattttgtcctttctttgtgtgcttgaaatattttatttttcagtttgtcctcatagggaatcaagtattttagctaggtgatgtcttgcaagtacgttccactttgttacaatctactatctgtatatactatttgtatcttaattcttttatgagatgttctgtaacatttttctcactttgacaaatgtttttagactgtacagtcaagatctggcgcttgggggtaagtggaatgatttgctaatattgagaatctgttgtatcaaacataataaactttttttgagatgtgaaaaaaaaaaaaaaaaaaaaa

SEQ ID NO: 95 - DNA, 1224 bp, Homo sapiens
atggagctgcttgaagaagatctcacatgccctatttgttgtagtctgtttgatgatccacgggttttgccttgctcccacaacttctgcaaaaaatgcttagaaggtatcttagaagggagtgtgcggaattccttgtggagaccagctccattcaagtgtcctacatgccgtaaggaaacttcagctactggaattaatagcctgcaggttaattactccctgaagggtattgtggaaaagtataacaagatcaagatctctcccaaaatgccagtatgcaaaggacacttggggcagcctctcaacattttctgcctgactgatatgcagctgatttgtgggatctgtgctactcgtggggagcacaccaaacatgtcttctgttctattgaagatgcctatgctcaggaaagggatgcctttgagtccctcttccagagctttgagacctggcgtcggggagatgctctttctcgcttggataccttggaaactagtaagaggaaatccctacagttactgactaaagattcagataaagtgaaggaattttttgagaagttacaacacacactggatcaaaagaagaatgaaattctgtctgactttgagaccatgaaacttgctgttatgcaagcatatgacccagagatcaacaaactcaacaccatcttgcaggagcaacggatggcctttaacattgctgaggctttcaaagatgtgtcagaacccattgtatttctgcaacagatgcaggagtttagagagaaaatcaaagtaatcaaggaaactcctttacctccctctaatttgcctgcaagccctttaatgaagaactttgataccagtcagtgggaagacataaaactagtcgatgtggataaactttctttgcctcaagacactggcacattcattagcaagattccctggagcttttataagttatttttgctaatccttctgcttggccttgtcattgtctttggtcctaccatgttcctagaatggtcattatttgatgacctggcaacttggaaaggctgtctttcaaacttcagttcctatctgactaaaacagccgatttcatagaacaatcagttttttactgggaacaggtgacagatgggtttttcattttcaatgaaagattcaagaattttactttggtggtactgaacaatgtggcagaatttgtgtgcaaatataaactattataa

SEQ ID NO: 96 - protein, 407 aa, Homo sapiens
MELLEEDLTCPICCSLFDDPRVLPCSHNFCKKCLEGILEGSVRNSLWRPAPFKCPTCRKETSATGINSLQVNYSLKGIVEKYNKIKISPKMPVCKGHLGQPLNIFCLTDMQLICGICATRGEHTKHVFCSIEDAYAQERDAFESLFQSFETWRRGDALSRLDTLETSKRKSLQLLTKDSDKVKEFFEKLQHTLDQKKNEILSDFETMKLAVMQAYDPEINKLNTILQEQRMAFNIAEAFKDVSEPIVFLQQMQEFREKIKVIKETPLPPSNLPASPLMKNFDTSQWEDIKLVDVDKLSLPQDTGTFISKIPWSFYKLFLLILLLGLVIVFGPTMFLEWSLFDDLATWKGCLSNFSSYLTKTADFIEQSVFYWEQVTDGFFIFNERFKNFTLVVLNNVAEFVCKYKLL

SEQ ID NO: 97 - DNA, 3740 bp, Homo sapiens
agacctacccgggcgaagcgggcgagcggtggtttggacgccggcggagacgcgggcgagtggttagcaggaagaagatgagccttaagtctgaacgccgaggaattcatgtggatcaatcggatctcctgtgcaagaaaggatgtggttactacggcaaccctgcctggcagggtttctgctccaagtgctggagggaagagtaccacaaagccaggcagaagcagattcaggaggactgggagctggcggagcgactccagcgggaggaagaagaggcctttgccagcagtcagagcagccaaggggcccaatccctcacattctccaagtttgaagaaaagaaaaccaacgagaagacccgcaaggttaccacagtgaagaaattcttcagtgcatcttccagggtcggatcaaagaaggaaattcaggaagcaaaagctcccagtccttccataaaccggcaaaccagcattgaaacggatagagtgtctaaggagttcatagaatttctcaagaccttccacaagacaggccaagaaatctataaacagaccaagctgtttttggaaggaatgcattacaaaagggatctaagcattgaagaacagtcagagtgtgctcaggatttctaccacaatgtggccgaaaggatgcaaactcgtgggaaagtgcctccagaaagagtcgagaagataatggatcagattgaaaagtacatcatgactcgtctctataaatatgtattctgtccagaaactactgatgatgagaagaaagatcttgccattcaaaagagaatcagagccctgcgctgggttacgcctcagatgctgtgtgtccctgttaatgaagacatcccagaagtgtctgatatggtggtgaaggcgatcacagatatcattgaaatggattccaagcgtgtgcctcgagacaagctggcctgcatcaccaagtgcagcaagcacatcttcaatgccatcaagatcaccaagaatgagccggcgtcagcggatgacttcctccccaccctcatctacattgttttgaagggcaaccccccacgccttcagtctaatatccagtatatcacgcgcttctgcaatccaagccgactgatgactggagaggatggctactatttcaccaatctgtgctgtgctgtggctttcattgagaagctagacgcccagtctttgaatctaagtcaggaggattttgatcgctacatgtctggccagacctctcccaggaagcaagaagctgagagttggtctcctgatgcttgcttaggcgtcaagcaaatgtataagaacttggatctcttgtctcagttgaatgaacgacaagaaaggatcatgaatgaagccaagaaactggaaaaagacctcatagattggacagatggaattgcaagagaagttcaagacatcgttgagaaatacccactggaaattaagcctccgaatcaaccgttagcagctattgactctgaaaacgttgaaaatgataaacttcctccaccactgcaacctcaagtttatgcaggatgatcacaatttagtggagagtatttatttgagcctaaattgtaggtagcccttactacactcaactgattgggatctagaatgtaactaaattgcttataaatgtcagagcattttttaaaggtacagtatatggggattgtttcgtttttcctagcaggggaaccttagttaataataaaatactacttatttgagttactgatacagattcatttaaggcttgtgtgcaaattttgtctcaatcttttttccctccatgattttcctatgtgcttcctctggcattcactgtggttttggtaaataattgccttttaaaggattaaacaaatgaatgctacaaagtgtatgttcaagaaaattaaatggtaccactcttccacagtttggaataattttataattgtaaagatagaaattatattgataagtaaatatgtaaaattgtaaatatgtaaaaaaaagaatggtgtctgctgtgcatggcattttatatgttaattttttagtttaaaatgaagtatattgaatgtttgcctttagcaccattttatttggtttgtcccactaaaatgactcgagaagtgtttagacaaactccccttaagatgtgcactccatctttaagaacgtgttagccttaactttgaggttctatatagtcagagactatgacaccactaagattcagaataaagtttaggccacataaaattgctgtttaatgtagtcgatggaagactttaaactatgcttctagcttatttttccctcattcattcagcaaatctctattgagttcttcagtgagaaggagcaggcactgggcctggaatggaaggcgggaatgaatgggcctctgatggtgagaggtgacggggtccctcagctgtgagatgcaaggggcgccttgcagcctccataatatacatttgactttgcaaacgtctagacatgttttctgaacctttttcaggacatttcaacctcgggactattcatattagtggcctgagaggtgtttgttgtggggccaccctgtgcatggtagaatgttcagcagcattcctgccatccatatccattaggtgccagtagcacccccgctagagctgtgaaaaattctctccagacatagtcagatgtctcctggggccatatcacccctccgttaagaaccactgatgtcttttacaaaccaggagttatcctcctggtggttaatatggtgtaaccaaagaatcttgcactcaatgcacagtgtgatgttaactaaaacgagttaaatatttaggaggcttgacagctacctgcattgtagaaccttttcttatctcagtggaaccttctataacctaaatataccattgatgattcttcttccattcagtgacatccacagattatgcagctatacttgtgaaatcgtgcatgaggccccagggcaccgttctagaacaacgtcacttcacacaggcagctgagaaaggttctcttgcttttccagtatcttcctaaggatggagcccaaaattgcagagcagtaactttggaataaaaccagggtgggtataaaacttcttattcttaaatttacatataagatctattaagcttgacacatctgtgtcatcacgcactgaagacaggaagcagttcactgagtcagctggttcccaagctcgcacagaaggtgataagttactatcaaatgccagtgagaatcttcttatagaataacctgggcccaagtgattttagtacaaaacttgcccttctttggtttaattttctatgtgcttttaggtgtgaatccagatatgcggtcttaattcctttggaaatacacagttcgtttagttactgtacactctgtttgttcaataaactgcatatcaacttcccacaaaagctgacttttttgggtctcttacatataaagtaggtcattgagttgatttttttggaggtatctcatattggtcgaattcttctggtatggactcttgccttatatagaggcttctatttctcttaagtcaccatcaatacaacggccggagtttctgtttttgcaataagaagatgttggtattttatgtagggtaaatgtgactggaatacacctttggaacggaattctttatcaataaagtttcacaatccgtccctcttccaaaaaaaaaa

SEQ ID NO: 98 - DNA, 1476 bp, Homo sapiens
atgagccttaagtctgaacgccgaggaattcatgtggatcaatcggatctcctgtgcaagaaaggatgtggttactacggcaaccctgcctggcagggtttctgctccaagtgctggagggaagagtaccacaaagccaggcagaagcagattcaggaggactgggagctggcggagcgactccagcgggaggaagaagaggcctttgccagcagtcagagcagccaaggggcccaatccctcacattctccaagtttgaagaaaagaaaaccaacgagaagacccgcaaggttaccacagtgaagaaattcttcagtgcatcttccagggtcggatcaaagaaggaaattcaggaagcaaaagctcccagtccttccataaaccggcaaaccagcattgaaacggatagagtgtctaaggagttcatagaatttctcaagaccttccacaagacaggccaagaaatctataaacagaccaagctgtttttggaaggaatgcattacaaaagggatctaagcattgaagaacagtcagagtgtgctcaggatttctaccacaatgtggccgaaaggatgcaaactcgtgggaaagtgcctccagaaagagtcgagaagataatggatcagattgaaaagtacatcatgactcgtctctataaatatgtattctgtccagaaactactgatgatgagaagaaagatcttgccattcaaaagagaatcagagccctgcgctgggttacgcctcagatgctgtgtgtccctgttaatgaagacatcccagaagtgtctgatatggtggtgaaggcgatcacagatatcattgaaatggattccaagcgtgtgcctcgagacaagctggcctgcatcaccaagtgcagcaagcacatcttcaatgccatcaagatcaccaagaatgagccggcgtcagcggatgacttcctccccaccctcatctacattgttttgaagggcaaccccccacgccttcagtctaatatccagtatatcacgcgcttctgcaatccaagccgactgatgactggagaggatggctactatttcaccaatctgtgctgtgctgtggctttcattgagaagctagacgcccagtctttgaatctaagtcaggaggattttgatcgctacatgtctggccagacctctcccaggaagcaagaagctgagagttggtctcctgatgcttgcttaggcgtcaagcaaatgtataagaacttggatctcttgtctcagttgaatgaacgacaagaaaggatcatgaatgaagccaagaaactggaaaaagacctcatagattggacagatggaattgcaagagaagttcaagacatcgttgagaaatacccactggaaattaagcctccgaatcaaccgttagcagctattgactctgaaaacgttgaaaatgataaacttcctccaccactgcaacctcaagtttatgcaggatga

SEQ ID NO: 99 - protein, 491 aa, Homo sapiens
MSLKSERRGIHVDQSDLLCKKGCGYYGNPAWQGFCSKCWREEYHKARQKQIQEDWELAERLQREEEEAFASSQSSQGAQSLTFSKFEEKKTNEKTRKVTTVKKFFSASSRVGSKKEIQEAKAPSPSINRQTSIETDRVSKEFIEFLKTFHKTGQEIYKQTKLFLEGMHYKRDLSIEEQSECAQDFYHNVAERMQTRGKVPPERVEKIMDQIEKYIMTRLYKYVFCPETTDDEKKDLAIQKRIRALRWVTPQMLCVPVNEDIPEVSDMVVKAITDIIEMDSKRVPRDKLACITKCSKHIFNAIKITKNEPASADDFLPTLIYIVLKGNPPRLQSNIQYITRFCNPSRLMTGEDGYYFTNLCCAVAFIEKLDAQSLNLSQEDFDRYMSGQTSPRKQEAESWSPDACLGVKQMYKNLDLLSQLNERQERIMNEAKKLEKDLIDWTDGIAREVQDIVEKYPLEIKPPNQPLAAIDSENVENDKLPPPLQPQVYAG

SEQ ID NO: 100 - DNA, 6527 bp, Homo sapiens
ggcgcgcatgcgtgcagctctttggaggcggtagctttttcggcgtcgagactggaggctgagtgctaaactgtgtggggcgcggatgggatccagctgttagtcgggtaggcatagctttgtgttattcttggaaaatttcgcaccacttgtgaattccttgaacctgggcattgcaaacccacttctgttgggcccatctcctttgcactttgctcagattaagactcagttggcgcttcagcagctgaatgccgttgcctcacatggttcaacaccaccttatactttattaaatcaggctttcttgaaaatagccatgtcgagacccaggtttaatcctcgaggagactttccacttcaaaggccacgagcacctaacccttctgggatgaggcctccaggaccatttatgaggcctggatctatgggtctcccaagattttacccagcagggagagcacgtggaattccacacagatttgctggccatgaatcttatcagaacatggggccacagagaatgaatgttcaggtaactcaacacagaactgatccaagattgaccaaagaaaaactggattttcatgaagcacaacagaagaaggggaagcctcatggtagccggtgggatgatgagcctcatatatctgcatcagtggcagtgaaacagagttctgtaacacaggttacagagcagagtcccaaagtacagagccgctatacaaaagagagtgcctcaagtatcttagcaagttttggattatctaatgaagacctagaagaacttagtcgctatcctgatgaacaactaactcctgaaaatatgccattaattttgagggatataagaatgcgaaaaatggggcgccgattacctaatttaccttctcagagcagaaataaagaaacacttggtagtgaagcagtttcaagtaatgtgatcgattatgggcatgcaagcaaatatggctacacagaagatccacttgaagtacgtatttatgatcctgaaattccaactgatgaggtcgagaatgaatttcagtcacagcagaacatttctgcatctgttcccaatccaaatgtgatatgtaattctatgtttcctgttgaagacgtatttcgccaaatggacttccccggtgagtcctccaataatcggtcctttttctcagttgagagtggaaccaagatgtcaggcttacacatttcaggaggacagtcagtccttgaacccataaaatccgtcaaccaatccattaaccaaacagttagccagacaatgagtcaatctctgattcctccatctatgaaccagcaacctttttcgtcggaattaatttcatctgtaagccagcaagagcggatcccacatgaacctgtgattaattcatctaacgtacatgttggatcaagaggaagtaaaaagaattaccagtcacaggctgacattcccattcggtctccctttggtattgtgaaagcatcctggctaccaaagttttcacatgctgatgcccagaagatgaagagacttccaactccttctatgatgaatgattattatgcagcatctccaagaatatttccacatttgtgttctctgtgtaacgtagaatgtagtcatttgaaggattggattcagcatcaaaatacatctactcatattgagagctgtcgacagttacgtcaacagtatcctgattggaatcctgagatcctcccatcgagaagaaatgagggcaatagaaaagaaaatgaaactccacgaagacgttctcattcccccagtcctaggcgttctagaagatcaagctcaagtcacagattccgtcggtctcgaagcccaatgcattacatgtataggccgagaagtcgaagtccaagaatttgccatcgtttcatttctagatacagatccagatccagatcccgttcaccatatcgaattagaaatccatttagaggtagtccaaaatgctttcgatcagttagccctgagaggatgtcaaggagatcagtgagatcatcagatagaaaaaaagcattagaagatgtagtacaacgatctgggcatgggacagaatttaataaacagaagcatcttgaagctgctgataagggacattcaccagcacaaaagcctaaaactagcagtggaacaaaaccatcagttaaacctacaagcgctacaaagagtgattcaaatctaggaggacattctattcgttgtaaatcaaagaatcttgaagatgacactttgtcagaatgtaaacaggtgtctgataaagctgtttctctccagcgaaagcttcggaaagaacagtcattgcattatggttcggttcttcttataactgaattaccagaggatggttgtactgaagaagatgtgagaaaattatttcaaccatttgggaaagtgaatgatgtcctaattgttccatatagaaaagaggcttacctagaaatggaatttaaagaggcaattactgcaattatgaagtacattgaaacaacacctcttacgataaaaggaaaaagtgtgaaaatatgtgttccaggaaagaaaaaagcacagaacaaagaggtgaagaaaaagactttagagtcaaagaaagtatctgcatctaccttaaaaagagatgcagatgcttcaaaagctgttgaaattgttacttcaacttctgctgccaaaactggacaagccaaggcatctgtagccaaagtaaacaaatctacagggaaatcagcaagttctgtaaaatctgtggtaacggtagctgttaaaggtaataaagcttcaatcaaaacagcaaaatctggtggaaagaagtctctagaagccaaaaagactgggaatgtcaaaaacaaagactctaacaaacctgtgactataccagaaaactctgaaataaagaccagtattgaagtcaaagccactgaaaactgtgctaaagaagctatttctgatgctgctttggaggccacagagaatgaaccacttaacaaggaaacagaagaaatgtgtgtgatgcttgtctctaatttgcctaataaaggatattctgtagaagaagtttatgacttagcaaaaccatttggtggtttaaaggatatcttgattttatcatctcataaaaaggcatatatagaaataaatagaaaagctgctgagtctatggtaaaattttatacctgcttcccagtattgatggatggaaatcaactctcaataagtatggctcctgaaaacatgaatataaaagatgaggaagctatatttataaccttggtaaaagaaaatgacccagaggcaaacatagatacaatttatgatcgatttgtacatcttgataatttaccggaagatggacttcagtgtgtactttgtgttggacttcagtttggaaaagtggatcaccatgtattcataagtaatagaaacaaggcaattcttcagttagatagtcctgaatctgctcagtcaatgtatagctttctgaaacaaaatccacaaaatattggtgaccatatgttgacctgctcattatctccaaagatagacttaccagaggtgcaaattgagcatgacccagaattagaaaaagaaagccctggcttgaaaaacagtccaattgatgaaagtgaggtgcaaacagcaactgatagtccctctgttaaacctaatgagcttgaagaagaaagtactcccagcattcaaacagaaactttggtacagcaggaagagccttgtgaggaagaagctgaaaaagcaacatgtgattctgactttgctgttgaaactttggagcttgaaactcaaggagaggaggtcaaagaagaaattcctcttgtagcatccgcttcagtcagtattgaacaattcactgaaaatgccgaggagtgtgctttaaatcagcagatgtttaacagtgacttggagaagaaaggggcagaaattattaaccctaaaacagcattgttaccatctgacagtgtgtttgcagaagaaaggaacctcaaaggaattctagaagaatctccatctgaagcagaagatttcatttctggaattacacagactatggtagaagctgtagctgaagtagaaaaaaatgaaactgtttcggaaatattgccatcaacttgtattgtgacgttagtaccaggaattcccactggggatgagaagacagtggacaaaaagaatatttctgaaaaaaaaggtaacatggatgaaaaggaggagaaggaatttaatactaaggaaaccagaatggatcttcaaataggaacagagaaggctgaaaagaatgaaggtaggatggatgcagaaaaggtggaaaagatggcagcaatgaaagaaaagcctgcagaaaacactttattcaaggcatacccaaataaaggagtgggtcaggctaataagcctgatgaaactagtaaaactagtattctggctgtatcagatgtatctagcagtaaaccaagcatcaaggctgttatagtctcttctcctaaggcaaaagctacagtttcaaaaactgaaaatcagaaaagttttccaaaatctgtgcccagagatcaaataaatgctgaaaagaaactttcagccaaggaatttggtctgcttaaacccacaagtgccaggtcaggcttggcagaaagcagcagtaaattcaaacctactcagagcagtcttaccagaggaggcagtggaaggatctcagccctgcaaggcaagctttctaaactggattacagagatataacaaaacaatctcaggaaacagaggctagaccttccatcatgaaacgggatgacagcaacaataagactttggctgagcaaaacactaagaatcctaaaagcactactggtagaagttccaaatctaaagaggagccattatttccatttaatttggatgaatttgttactgtggatgaggttatagaagaagtgaatccttctcaggccaagcagaatccactaaagggaaaaaggaaagaaactctcaaaaatgttcctttctctgaacttaacttaaagaagaaaaaggggaaaacttccactcctcgtggtgttgagggagaactatcttttgtgacattggatgagattggggaagaggaagatgcagctgcacatctagcacaagctctagtcactgtggatgaagtaattgatgaagaagaactaaatatggaagaaatggtaaaaaattcaaattcactttttacattagatgaattaattgaccaagatgattgcatttcccacagtgaacctaaagatgttactgttctgtcagtggctgaagaacaagatctcctcaaacaggaacgcttggtaactgtggatgaaattggagaagtggaagagctacctttgaatgagtcagcagacataacttttgccactttaaatactaaaggaaatgaaggagatactgtaagggattccattggcttcatttcttctcaggtgcccgaagacccttctactttagttactgtagatgaaatacaagatgacagcagtgatttgcatttagtgactttggatgaagtaactgaagaggatgaagactctctggcggattttaacaaccttaaagaagagcttaattttgttactgttgatgaagttggagaggaggaagatggagataatgatttaaaagttgagttagcacaaagcaaaaatgaccatcccacagataaaaaagggaatagaaagaagagagctgtggacacaaaaaagacaaaacttgaatccttgtcccaagtgggtccagtaaatgagaatgttatggaagaagatctaaaaaccatgattgaaagacacttaacagctaaaactccaaccaagagagttagaattgggaaaactctgccatcagaaaaagctgttgtgacagaaccagcaaaaggtgaagaggccttccagatgagtgaagttgatgaggaatctggattaaaggattcagaaccagagcgaaaacgcaagaagactgaagactcttcttcaggcaaatcagtggcgtctgatgtccctgaggaattagactttcttgtacctaaggctggattcttctgtccaatttgttccctcttctactcaggtgaaaaagcaatgacaaatcactgcaagagtacacgtcataagcaaaatactgagaaattcatggccaagcaaagaaaggaaaaggagcagaatgaggctgaagaaagaagctctaggtgattgggggaaaggaaagaattcactagaaatttgtttagggtccagttgatttgtgtatttttgttatcatttaatttgtaattttcgtttcagaagcaaatattcgtgttgtacaaatttctgattgccctaaatgtagagagactgatggggaaagtatgatgggtttgatttttatatcaaatcatcaggcatggagaaatatcttttagaagtgttaaaataaatgttcctactgtatatttaaaatacaaaaaaaaaaaaaaaaaaa

SEQ ID NO: 101 - DNA, 5937 bp, Homo sapiens
atgtcgagacccaggtttaatcctcgaggagactttccacttcaaaggccacgagcacctaacccttctgggatgaggcctccaggaccatttatgaggcctggatctatgggtctcccaagattttacccagcagggagagcacgtggaattccacacagatttgctggccatgaatcttatcagaacatggggccacagagaatgaatgttcaggtaactcaacacagaactgatccaagattgaccaaagaaaaactggattttcatgaagcacaacagaagaaggggaagcctcatggtagccggtgggatgatgagcctcatatatctgcatcagtggcagtgaaacagagttctgtaacacaggttacagagcagagtcccaaagtacagagccgctatacaaaagagagtgcctcaagtatcttagcaagttttggattatctaatgaagacctagaagaacttagtcgctatcctgatgaacaactaactcctgaaaatatgccattaattttgagggatataagaatgcgaaaaatggggcgccgattacctaatttaccttctcagagcagaaataaagaaacacttggtagtgaagcagtttcaagtaatgtgatcgattatgggcatgcaagcaaatatggctacacagaagatccacttgaagtacgtatttatgatcctgaaattccaactgatgaggtcgagaatgaatttcagtcacagcagaacatttctgcatctgttcccaatccaaatgtgatatgtaattctatgtttcctgttgaagacgtatttcgccaaatggacttccccggtgagtcctccaataatcggtcctttttctcagttgagagtggaaccaagatgtcaggcttacacatttcaggaggacagtcagtccttgaacccataaaatccgtcaaccaatccattaaccaaacagttagccagacaatgagtcaatctctgattcctccatctatgaaccagcaacctttttcgtcggaattaatttcatctgtaagccagcaagagcggatcccacatgaacctgtgattaattcatctaacgtacatgttggatcaagaggaagtaaaaagaattaccagtcacaggctgacattcccattcggtctccctttggtattgtgaaagcatcctggctaccaaagttttcacatgctgatgcccagaagatgaagagacttccaactccttctatgatgaatgattattatgcagcatctccaagaatatttccacatttgtgttctctgtgtaacgtagaatgtagtcatttgaaggattggattcagcatcaaaatacatctactcatattgagagctgtcgacagttacgtcaacagtatcctgattggaatcctgagatcctcccatcgagaagaaatgagggcaatagaaaagaaaatgaaactccacgaagacgttctcattcccccagtcctaggcgttctagaagatcaagctcaagtcacagattccgtcggtctcgaagcccaatgcattacatgtataggccgagaagtcgaagtccaagaatttgccatcgtttcatttctagatacagatccagatccagatcccgttcaccatatcgaattagaaatccatttagaggtagtccaaaatgctttcgatcagttagccctgagaggatgtcaaggagatcagtgagatcatcagatagaaaaaaagcattagaagatgtagtacaacgatctgggcatgggacagaatttaataaacagaagcatcttgaagctgctgataagggacattcaccagcacaaaagcctaaaactagcagtggaacaaaaccatcagttaaacctacaagcgctacaaagagtgattcaaatctaggaggacattctattcgttgtaaatcaaagaatcttgaagatgacactttgtcagaatgtaaacaggtgtctgataaagctgtttctctccagcgaaagcttcggaaagaacagtcattgcattatggttcggttcttcttataactgaattaccagaggatggttgtactgaagaagatgtgagaaaattatttcaaccatttgggaaagtgaatgatgtcctaattgttccatatagaaaagaggcttacctagaaatggaatttaaagaggcaattactgcaattatgaagtacattgaaacaacacctcttacgataaaaggaaaaagtgtgaaaatatgtgttccaggaaagaaaaaagcacagaacaaagaggtgaagaaaaagactttagagtcaaagaaagtatctgcatctaccttaaaaagagatgcagatgcttcaaaagctgttgaaattgttacttcaacttctgctgccaaaactggacaagccaaggcatctgtagccaaagtaaacaaatctacagggaaatcagcaagttctgtaaaatctgtggtaacggtagctgttaaaggtaataaagcttcaatcaaaacagcaaaatctggtggaaagaagtctctagaagccaaaaagactgggaatgtcaaaaacaaagactctaacaaacctgtgactataccagaaaactctgaaataaagaccagtattgaagtcaaagccactgaaaactgtgctaaagaagctatttctgatgctgctttggaggccacagagaatgaaccacttaacaaggaaacagaagaaatgtgtgtgatgcttgtctctaatttgcctaataaaggatattctgtagaagaagtttatgacttagcaaaaccatttggtggtttaaaggatatcttgattttatcatctcataaaaaggcatatatagaaataaatagaaaagctgctgagtctatggtaaaattttatacctgcttcccagtattgatggatggaaatcaactctcaataagtatggctcctgaaaacatgaatataaaagatgaggaagctatatttataaccttggtaaaagaaaatgacccagaggcaaacatagatacaatttatgatcgatttgtacatcttgataatttaccggaagatggacttcagtgtgtactttgtgttggacttcagtttggaaaagtggatcaccatgtattcataagtaatagaaacaaggcaattcttcagttagatagtcctgaatctgctcagtcaatgtatagctttctgaaacaaaatccacaaaatattggtgaccatatgttgacctgctcattatctccaaagatagacttaccagaggtgcaaattgagcatgacccagaattagaaaaagaaagccctggcttgaaaaacagtccaattgatgaaagtgaggtgcaaacagcaactgatagtccctctgttaaacctaatgagcttgaagaagaaagtactcccagcattcaaacagaaactttggtacagcaggaagagccttgtgaggaagaagctgaaaaagcaacatgtgattctgactttgctgttgaaactttggagcttgaaactcaaggagaggaggtcaaagaagaaattcctcttgtagcatccgcttcagtcagtattgaacaattcactgaaaatgccgaggagtgtgctttaaatcagcagatgtttaacagtgacttggagaagaaaggggcagaaattattaaccctaaaacagcattgttaccatctgacagtgtgtttgcagaagaaaggaacctcaaaggaattctagaagaatctccatctgaagcagaagatttcatttctggaattacacagactatggtagaagctgtagctgaagtagaaaaaaatgaaactgtttcggaaatattgccatcaacttgtattgtgacgttagtaccaggaattcccactggggatgagaagacagtggacaaaaagaatatttctgaaaaaaaaggtaacatggatgaaaaggaggagaaggaatttaatactaaggaaaccagaatggatcttcaaataggaacagagaaggctgaaaagaatgaaggtaggatggatgcagaaaaggtggaaaagatggcagcaatgaaagaaaagcctgcagaaaacactttattcaaggcatacccaaataaaggagtgggtcaggctaataagcctgatgaaactagtaaaactagtattctggctgtatcagatgtatctagcagtaaaccaagcatcaaggctgttatagtctcttctcctaaggcaaaagctacagtttcaaaaactgaaaatcagaaaagttttccaaaatctgtgcccagagatcaaataaatgctgaaaagaaactttcagccaaggaatttggtctgcttaaacccacaagtgccaggtcaggcttggcagaaagcagcagtaaattcaaacctactcagagcagtcttaccagaggaggcagtggaaggatctcagccctgcaaggcaagctttctaaactggattacagagatataacaaaacaatctcaggaaacagaggctagaccttccatcatgaaacgggatgacagcaacaataagactttggctgagcaaaacactaagaatcctaaaagcactactggtagaagttccaaatctaaagaggagccattatttccatttaatttggatgaatttgttactgtggatgaggttatagaagaagtgaatccttctcaggccaagcagaatccactaaagggaaaaaggaaagaaactctcaaaaatgttcctttctctgaacttaacttaaagaagaaaaaggggaaaacttccactcctcgtggtgttgagggagaactatcttttgtgacattggatgagattggggaagaggaagatgcagctgcacatctagcacaagctctagtcactgtggatgaagtaattgatgaagaagaactaaatatggaagaaatggtaaaaaattcaaattcactttttacattagatgaattaattgaccaagatgattgcatttcccacagtgaacctaaagatgttactgttctgtcagtggctgaagaacaagatctcctcaaacaggaacgcttggtaactgtggatgaaattggagaagtggaagagctacctttgaatgagtcagcagacataacttttgccactttaaatactaaaggaaatgaaggagatactgtaagggattccattggcttcatttcttctcaggtgcccgaagacccttctactttagttactgtagatgaaatacaagatgacagcagtgatttgcatttagtgactttggatgaagtaactgaagaggatgaagactctctggcggattttaacaaccttaaagaagagcttaattttgttactgttgatgaagttggagaggaggaagatggagataatgatttaaaagttgagttagcacaaagcaaaaatgaccatcccacagataaaaaagggaatagaaagaagagagctgtggacacaaaaaagacaaaacttgaatccttgtcccaagtgggtccagtaaatgagaatgttatggaagaagatctaaaaaccatgattgaaagacacttaacagctaaaactccaaccaagagagttagaattgggaaaactctgccatcagaaaaagctgttgtgacagaaccagcaaaaggtgaagaggccttccagatgagtgaagttgatgaggaatctggattaaaggattcagaaccagagcgaaaacgcaagaagactgaagactcttcttcaggcaaatcagtggcgtctgatgtccctgaggaattagactttcttgtacctaaggctggattcttctgtccaatttgttccctcttctactcaggtgaaaaagcaatgacaaatcactgcaagagtacacgtcataagcaaaatactgagaaattcatggccaagcaaagaaaggaaaaggagcagaatgaggctgaagaaagaagctctaggtga

SEQ ID NO: 102 - protein, 1978 aa, Homo sapiens
MSRPRFNPRGDFPLQRPRAPNPSGMRPPGPFMRPGSMGLPRFYPAGRARGIPHRFAGHESYQNMGPQRMNVQVTQHRTDPRLTKEKLDFHEAQQKKGKPHGSRWDDEPHISASVAVKQSSVTQVTEQSPKVQSRYTKESASSILASFGLSNEDLEELSRYPDEQLTPENMPLILRDIRMRKMGRRLPNLPSQSRNKETLGSEAVSSNVIDYGHASKYGYTEDPLEVRIYDPEIPTDEVENEFQSQQNISASVPNPNVICNSMFPVEDVFRQMDFPGESSNNRSFFSVESGTKMSGLHISGGQSVLEPIKSVNQSINQTVSQTMSQSLIPPSMNQQPFSSELISSVSQQERIPHEPVINSSNVHVGSRGSKKNYQSQADIPIRSPFGIVKASWLPKFSHADAQKMKRLPTPSMMNDYYAASPRIFPHLCSLCNVECSHLKDWIQHQNTSTHIESCRQLRQQYPDWNPEILPSRRNEGNRKENETPRRRSHSPSPRRSRRSSSSHRFRRSRSPMHYMYRPRSRSPRICHRFISRYRSRSRSRSPYRIRNPFRGSPKCFRSVSPERMSRRSVRSSDRKKALEDVVQRSGHGTEFNKQKHLEAADKGHSPAQKPKTSSGTKPSVKPTSATKSDSNLGGHSIRCKSKNLEDDTLSECKQVSDKAVSLQRKLRKEQSLHYGSVLLITELPEDGCTEEDVRKLFQPFGKVNDVLIVPYRKEAYLEMEFKEAITAIMKYIETTPLTIKGKSVKICVPGKKKAQNKEVKKKTLESKKVSASTLKRDADASKAVEIVTSTSAAKTGQAKASVAKVNKSTGKSASSVKSVVTVAVKGNKASIKTAKSGGKKSLEAKKTGNVKNKDSNKPVTIPENSEIKTSIEVKATENCAKEAISDAALEATENEPLNKETEEMCVMLVSNLPNKGYSVEEVYDLAKPFGGLKDILILSSHKKAYIEINRKAAESMVKFYTCFPVLMDGNQLSISMAPENMNIKDEEAIFITLVKENDPEANIDTIYDRFVHLDNLPEDGLQCVLCVGLQFGKVDHHVFISNRNKAILQLDSPESAQSMYSFLKQNPQNIGDHMLTCSLSPKIDLPEVQIEHDPELEKESPGLKNSPIDESEVQTATDSPSVKPNELEEESTPSIQTETLVQQEEPCEEEAEKATCDSDFAVETLELETQGEEVKEEIPLVASASVSIEQFTENAEECALNQQMFNSDLEKKGAEIINPKTALLPSDSVFAEERNLKGILEESPSEAEDFISGITQTMVEAVAEVEKNETVSEILPSTCIVTLVPGIPTGDEKTVDKKNISEKKGNMDEKEEKEFNTKETRMDLQIGTEKAEKNEGRMDAEKVEKMAAMKEKPAENTLFKAYPNKGVGQANKPDETSKTSILAVSDVSSSKPSIKAVIVSSPKAKATVSKTENQKSFPKSVPRDQINAEKKLSAKEFGLLKPTSARSGLAESSSKFKPTQSSLTRGGSGRISALQGKLSKLDYRDITKQSQETEARPSIMKRDDSNNKTLAEQNTKNPKSTTGRSSKSKEEPLFPFNLDEFVTVDEVIEEVNPSQAKQNPLKGKRKETLKNVPFSELNLKKKKGKTSTPRGVEGELSFVTLDEIGEEEDAAAHLAQALVTVDEVIDEEELNMEEMVKNSNSLFTLDELIDQDDCISHSEPKDVTVLSVAEEQDLLKQERLVTVDEIGEVEELPLNESADITFATLNTKGNEGDTVRDSIGFISSQVPEDPSTLVTVDEIQDDSSDLHLVTLDEVTEEDEDSLADFNNLKEELNFVTVDEVGEEEDGDNDLKVELAQSKNDHPTDKKGNRKKRAVDTKKTKLESLSQVGPVNENVMEEDLKTMIERHLTAKTPTKRVRIGKTLPSEKAVVTEPAKGEEAFQMSEVDEESGLKDSEPERKRKKTEDSSSGKSVASDVPEELDFLVPKAGFFCPICSLFYSGEKAMTNHCKSTRHKQNTEKFMAKQRKEKEQNEAEERSSR

SEQ ID NO: 103 - DNA, 2509 bp, Homo sapiens
gtctcaaatttgcagggagggacggggtggggggtggggggaccccggttgtgcagtttgatattgagggagcccccacctactcgctggggctgcgtaatctggacgtttccaaactgaagcgaaggcgtcgggagactaggcctcagagaaccatggctactgccaagggaatcgccataggcatcgacctgggcaccacctactcctgtgtgggggtgttccagcacggcaaggtggagatcatcgccaacgaccagggcaaccgcaccacccccagctacgtggccttcacagacaccgagcggctcattggggatgcggccaagaaccaggtagcaatgaatccccagaacactgtttttgatgctaaacgtctgatcggcaggaaatttaatgatcctgttgtacaagcagatatgaaactttggccttttcaagtgattaatgaaggaggcaagcccaaagtccttgtgtcctacaaaggggagaataaagctttctaccctgaggaaatctcttcgatggtattgactaagttgaaggagactgctgaggcctttttgggccaccctgtcaccaatgcagtgattaccgtgccagcctatttcaatgactctcaacgtcaggctactaaggatgcaggtgtgattgctggacttaatgtgctaagaatcatcaatgagcccacggctgctgccattgcctatggtttagataaaggaggtcaaggagaacgacatgtcctgatttttgatctgggtggaggcacatttgatgtgtcaattctgaccatagatgatgggatttttgaggtaaaggccactgctggggacactcacctgggtggggaggactttgacaacaggcttgtgagccacttcgtggaggagttcaagaggaaacacaaaaaggacatcagccagaacaagcgagccgtgaggcggctgcgcaccgcctgcgagagggccaagaggaccctgtcgtccagcacccaggccaacctagaaattgattcactttatgaaggcattgacttctatacatccatcaccagagctcgatttgaagagttgtgtgcagacctgtttaggggtaccctggagcctgtagaaaaagcgcttcgggatgccaagatggataaggctaaaatccatgacattgttttagtagggggctccacccgcatccccaaggtgcagcggctgcttcaggactacttcaatggacgtgatctcaacaagagcatcaaccctgatgaggccgtagcatatggggctgcggtacaagcagccatcctgatgggggacaagtctgagaaggtacaggacctgctgctgctggacgtggctcccctgtccctggggctggagacggctgggggcgtgatgactgccctgataaagcgcaactccaccatccccaccaagcagacacagattttcaccacctactctgacaaccaacccggggtgctgatccaggtgtatgagggcgagagggccatgacaaaggacaacaacctgctggggcggtttgacctgactggaatccctccagcacccaggggagttcctcagatcgaggtgacgtttgacattgatgccaatggtattctcaatgtcacagccatggacaagagcaccggcaaggtgaacaagatcaccatcaccaatgacaagggccgcctgagcaaggaggagattgagcgcatggttctggatgctgagaaatataaagctgaagatgaggtccagagggagaaaattgctgcaaagaatgccttagaatcctatgcttttaacatgaagagtgttgtgagtgatgaaggtttgaagggcaagattagtgagtctgataaaaataaaatattggataaatgcaacgagctcctttcgtggctggaggtcaatcaactggcagagaaagatgagtttgatcataagagaaaggaattggagcagatgtgtaaccctatcatcacaaaactctaccaaggaggatgcactgggcctgcctgcggaacagggtatgtgcctggaaggcctgccacaggccccacaattgaagaagtagattaattctttttagaactgaagcatcctaggatgcctctacatgtatttcattcccctcatcttcaaacatcattattattcttgaccagacctgaatctaagttaccatcccttggaaattctggagaaggagtctcatgcaccacctatcacactccctcacatcctgtttctgactttggaatggactcaggaaaactaggcccctctttaaaccgtgtgatgtatttgaatgtctgttatttccagccaccctaacattcttcttcctgtgtggatgcttatttgtcaatcagtaaatttgttcgtaaagaaaattacttctggtatttaggctgtgaatgtaccttgaaggggagagttcatggagagagcatgtgttctctgattgtgaggtcactgtgaatgattaaattggtaagggtaaagta

SEQ ID NO: 104 - DNA, 1926 bp, Homo sapiens
atggctactgccaagggaatcgccataggcatcgacctgggcaccacctactcctgtgtgggggtgttccagcacggcaaggtggagatcatcgccaacgaccagggcaaccgcaccacccccagctacgtggccttcacagacaccgagcggctcattggggatgcggccaagaaccaggtagcaatgaatccccagaacactgtttttgatgctaaacgtctgatcggcaggaaatttaatgatcctgttgtacaagcagatatgaaactttggccttttcaagtgattaatgaaggaggcaagcccaaagtccttgtgtcctacaaaggggagaataaagctttctaccctgaggaaatctcttcgatggtattgactaagttgaaggagactgctgaggcctttttgggccaccctgtcaccaatgcagtgattaccgtgccagcctatttcaatgactctcaacgtcaggctactaaggatgcaggtgtgattgctggacttaatgtgctaagaatcatcaatgagcccacggctgctgccattgcctatggtttagataaaggaggtcaaggagaacgacatgtcctgatttttgatctgggtggaggcacatttgatgtgtcaattctgaccatagatgatgggatttttgaggtaaaggccactgctggggacactcacctgggtggggaggactttgacaacaggcttgtgagccacttcgtggaggagttcaagaggaaacacaaaaaggacatcagccagaacaagcgagccgtgaggcggctgcgcaccgcctgcgagagggccaagaggaccctgtcgtccagcacccaggccaacctagaaattgattcactttatgaaggcattgacttctatacatccatcaccagagctcgatttgaagagttgtgtgcagacctgtttaggggtaccctggagcctgtagaaaaagcgcttcgggatgccaagatggataaggctaaaatccatgacattgttttagtagggggctccacccgcatccccaaggtgcagcggctgcttcaggactacttcaatggacgtgatctcaacaagagcatcaaccctgatgaggccgtagcatatggggctgcggtacaagcagccatcctgatgggggacaagtctgagaaggtacaggacctgctgctgctggacgtggctcccctgtccctggggctggagacggctgggggcgtgatgactgccctgataaagcgcaactccaccatccccaccaagcagacacagattttcaccacctactctgacaaccaacccggggtgctgatccaggtgtatgagggcgagagggccatgacaaaggacaacaacctgctggggcggtttgacctgactggaatccctccagcacccaggggagttcctcagatcgaggtgacgtttgacattgatgccaatggtattctcaatgtcacagccatggacaagagcaccggcaaggtgaacaagatcaccatcaccaatgacaagggccgcctgagcaaggaggagattgagcgcatggttctggatgctgagaaatataaagctgaagatgaggtccagagggagaaaattgctgcaaagaatgccttagaatcctatgcttttaacatgaagagtgttgtgagtgatgaaggtttgaagggcaagattagtgagtctgataaaaataaaatattggataaatgcaacgagctcctttcgtggctggaggtcaatcaactggcagagaaagatgagtttgatcataagagaaaggaattggagcagatgtgtaaccctatcatcacaaaactctaccaaggaggatgcactgggcctgcctgcggaacagggtatgtgcctggaaggcctgccacaggccccacaattgaagaagtagattaa

SEQ ID NO: 105 - protein, 641 aa, Homo sapiens
MATAKGIAIGIDLGTTYSCVGVFQHGKVEIIANDQGNRTTPSYVAFTDTERLIGDAAKNQVAMNPQNTVFDAKRLIGRKFNDPVVQADMKLWPFQVINEGGKPKVLVSYKGENKAFYPEEISSMVLTKLKETAEAFLGHPVTNAVITVPAYFNDSQRQATKDAGVIAGLNVLRIINEPTAAAIAYGLDKGGQGERHVLIFDLGGGTFDVSILTIDDGIFEVKATAGDTHLGGEDFDNRLVSHFVEEFKRKHKKDISQNKRAVRRLRTACERAKRTLSSSTQANLEIDSLYEGIDFYTSITRARFEELCADLFRGTLEPVEKALRDAKMDKAKIHDIVLVGGSTRIPKVQRLLQDYFNGRDLNKSINPDEAVAYGAAVQAAILMGDKSEKVQDLLLLDVAPLSLGLETAGGVMTALIKRNSTIPTKQTQIFTTYSDNQPGVLIQVYEGERAMTKDNNLLGRFDLTGIPPAPRGVPQIEVTFDIDANGILNVTAMDKSTGKVNKITITNDKGRLSKEEIERMVLDAEKYKAEDEVQREKIAAKNALESYAFNMKSVVSDEGLKGKISESDKNKILDKCNELLSWLEVNQLAEKDEFDHKRKELEQMCNPIITKLYQGGCTGPACGTGYVPGRPATGPTIEEVD

SEQ ID NO: 106 - DNA, 661 bp, Homo sapiens
gagcctccggccgcccgccgggtttgtcccgcgatccccgaccatgcccgccgacctcagcggtacttggaccctgctcagcagcgacaacttcgagggctacatgctggccctaggtattgactttgccactcgtaaaatagccaagttgctgaagccacagaaagtgattgagcagaatggggattcttttaccatccacacgaacagcagcctaaggaactactttgtgaaatttaaagttggagaagaatttgatgaagataacagaggcctggacaacagaaaatgcaagagtttggttatctgggacaatgacaggctcacctgtatccagaagggagaaaagaagaacagaggctggacccattggatcgaaggagacaaactccacctggaaatgttctgtgaaggtcaagtgtgcaaacagacattccagagagcctgatccacatccagcagcagagcccacttgtggctgcagctttatgccaaattatattgcagactgaacagacgtttatctatcccatttggcgacgaggactcgtggctggagagagccacacagcgtgtaacctgaagtcatctagattatggggaaactgctcagcttcaataaacctgtccacaatgaaaaaaaaaaaaaaaaaaaaaaaaa

SEQ ID NO: 107 - DNA, 405 bp, Homo sapiens
atgcccgccgacctcagcggtacttggaccctgctcagcagcgacaacttcgagggctacatgctggccctaggtattgactttgccactcgtaaaatagccaagttgctgaagccacagaaagtgattgagcagaatggggattcttttaccatccacacgaacagcagcctaaggaactactttgtgaaatttaaagttggagaagaatttgatgaagataacagaggcctggacaacagaaaatgcaagagtttggttatctgggacaatgacaggctcacctgtatccagaagggagaaaagaagaacagaggctggacccattggatcgaaggagacaaactccacctggaaatgttctgtgaaggtcaagtgtgcaaacagacattccagagagcctga

SEQ ID NO: 108 - protein, 134 aa, Homo sapiens
MPADLSGTWTLLSSDNFEGYMLALGIDFATRKIAKLLKPQKVIEQNGDSFTIHTNSSLRNYFVKFKVGEEFDEDNRGLDNRKCKSLVIWDNDRLTCIQKGEKKNRGWTHWIEGDKLHLEMFCEGQVCKQTFQRA

SEQ ID NO: 109 - DNA, 1345 bp, Homo sapiens
aaaacagccggggctccagcgggagaacgataatgcaaagtgctatgttcttggctgttcaacacgactgcagacccatggacaagagcgcaggcagtggccacaagagcgaggagaagcgagaaaagatgaaacggacccttttaaaagattggaagacccgtttgagctacttcttacaaaattcctctactcctgggaagcccaaaaccggcaaaaaaagcaaacagcaagctttcatcaagccttctcctgaggaagcacagctgtggtcagaagcatttgacgagctgctagccagcaaatatggtcttgctgcattcagggcttttttaaagtcggaattctgtgaagaaaatattgaattctggctggcctgtgaagacttcaaaaaaaccaaatcaccccaaaagctgtcctcaaaagcaaggaaaatatatactgacttcatagaaaaggaagctccaaaagagataaacatagattttcaaaccaaaactctgattgcccagaatatacaagaagctacaagtggctgctttacaactgcccagaaaagggtatacagcttgatggagaacaactcttatcctcgtttcttggagtcagaattctaccaggacttgtgtaaaaagccacaaatcaccacagagcctcatgctacatgaaatgtaaaagggagcccagaaatggaggacatttcattctttttcctgaggggaaggactgtgacctgccataaagactgaccttgaattcagcctgggtgttcaggaaacatcactcagaactattgattcaaagttgggtagtgaatcaggaagccagtaactgactaggagaagctggtatcagaacagcttccctcactgtgtacagaacgcaagaagggaataggtggtctgaacgtggtgtctcactctgaaaagcaggaatgtaagatgatgaaagagacaatgtaatactgttggtccaaaagcatttaaaatcaatagatctgggattatgtggccttaggtagctggttgtacatctttccctaaatcgatccatgttaccacatagtagttttagtttaggattcagtaacagtgaagtgtttactatgtgcaagggtattgaagttcttatgaccacagatcatcagtactgttgtctcatgtaatgctaaaactgaaatggtccgtgtttgcattgttaaaaatgatgtgtgaaatagaatgagtgctatggtgttgaaaactgcagtgtccgttatgagtgccaaaaatctgtcttgaaggcagctacactttgaagtggtctttgaatacttttaataaatttattttgataaataatattg

SEQ ID NO: 110 - DNA, 636 bp, Homo sapiens
atgcaaagtgctatgttcttggctgttcaacacgactgcagacccatggacaagagcgcaggcagtggccacaagagcgaggagaagcgagaaaagatgaaacggacccttttaaaagattggaagacccgtttgagctacttcttacaaaattcctctactcctgggaagcccaaaaccggcaaaaaaagcaaacagcaagctttcatcaagccttctcctgaggaagcacagctgtggtcagaagcatttgacgagctgctagccagcaaatatggtcttgctgcattcagggcttttttaaagtcggaattctgtgaagaaaatattgaattctggctggcctgtgaagacttcaaaaaaaccaaatcaccccaaaagctgtcctcaaaagcaaggaaaatatatactgacttcatagaaaaggaagctccaaaagagataaacatagattttcaaaccaaaactctgattgcccagaatatacaagaagctacaagtggctgctttacaactgcccagaaaagggtatacagcttgatggagaacaactcttatcctcgtttcttggagtcagaattctaccaggacttgtgtaaaaagccacaaatcaccacagagcctcatgctacatga

SEQ ID NO: 111 - protein, 211 aa, Homo sapiens
MQSAMFLAVQHDCRPMDKSAGSGHKSEEKREKMKRTLLKDWKTRLSYFLQNSSTPGKPKTGKKSKQQAFIKPSPEEAQLWSEAFDELLASKYGLAAFRAFLKSEFCEENIEFWLACEDFKKTKSPQKLSSKARKIYTDFIEKEAPKEINIDFQTKTLIAQNIQEATSGCFTTAQKRVYSLMENNSYPRFLESEFYQDLCKKPQITTEPHAT

SEQ ID NO: 112 - DNA, 2273 bp, Homo sapiens
caggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataacattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttgctatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctaatttgccagtgggaactcctaaatcaaattggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgataaaag

SEQ ID NO: 113 - DNA, 1125 bp, Homo sapiens
atgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataacattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctag

SEQ ID NO: 114 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 115 - DNA, 3686 bp, Homo sapiens
atacgggagaactaaggctgaaacctcggaggaacaaccacttttgaagtgacttcgcggcgtgcgttgggtgcggactaggtggccgcggcgggagtgtgctggagcctgaagtccacgcgcgcggctgagaaccgccgggaccgcacgtgggcgccgcgcgcttcccccgcttcccaggtgggcgccggccgccaggccacctcacgtccggccccggggatgcgcgtcctcctcgccgcgctgggactgctgttcctgggggcgctacgagccttcccacaggatcgacccttcgaggacacctgtcatggaaaccccagccactactatgacaaggctgtcaggaggtgctgttaccgctgccccatggggctgttcccgacacagcagtgcccacagaggcctactgactgcaggaagcagtgtgagcctgactactacctggatgaggccgaccgctgtacagcctgcgtgacttgttctcgagacgacctcgtggagaagacgccgtgtgcatggaactcctcccgtgtctgcgaatgtcgacccggcatgttctgttccacgtctgccgtcaactcctgtgcccgctgcttcttccattctgtctgtccggcagggatgattgtcaagttcccaggcacggcgcagaagaacacggtctgtgagccggcttccccaggggtcagccctgcctgtgccagcccagagaactgcaaggaaccctccagtggcaccatcccccaggccaagcccaccccggtgtccccagcaacctccagtgccagcaccatgcctgtaagagggggcacccgcctcgcccaggaagctgcttctaaactgacgagggctcccgactctccctcctctgtgggaaggcctagttcagatccaggtctgtccccaacacagccatgcccagaggggtctggtgattgcagaaagcagtgtgagcccgactactacctggacgaggccggccgctgcacggcctgcgtgagctgttctcgagatgaccttgtggagaagacgccatgtgcatggaactcctcccgcacctgcgaatgtcgacctggcatgatctgtgccacatcagccaccaactcccgtgcccgctgtgtcccctacccaatctgtgcagcagagacggtcaccaagccccaggatatggctgagaaggacaccacctttgaggcgccacccctggggacccagccggactgcaaccccaccccagagaatggcgaggcgcctgccagcaccagccccactcagagcttgctggtggactcccaggccagtaagacgctgcccatcccaaccagcgctcccgtcgctctctcctccacggggaagcccgttctggatgcagggccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagtgaggcctgggctgggctggggctaggagggcagcagggtggcctctgggaggccaggatggcactgttggcaccgaggttgggggcagaggcccatctggcctgaactgaggctccagcatctagtggtggaccggccggtcactgcaggggtctggtggtctctgcttgcatccccaacttagctgtcccctgacccagagcctaggggatccggggcttgtacagaagagacagtccaaggggactggatcccagcagtgatgttggttgaggcagcaaacagatggcaggatgggcactgccgagaacagcattggtcccagagccctgggcatcagaccttaaccaccaggcccacagcccagcgagggagaggtcgtgaggccagctcccggggcccctgtaaccctactctcctctctccctggacctcagaggtgacacccattgggcccttccggcatgcccccagttactgtaaatgtggcccccagtgggcatggagccagtgcctgtggttgtttctccagagtcaaaagggaagtcgagggatggggcgtcgtcagctggcactgtctctgctgcagcggccacactgtactctgcactggtgtgagggcccctgcctggactgtgggaccctcctggtgctgcccaccttccctgtcctgtagccccctcggtgggcccagggcctagggcccaggatcaagtcactcatctcagaatgtccccaccaatccccgccacagcaggcgcctcgggtcccagatgtctgcagccctcagcagctgcagaccgcccctcaccaacccagagaacctgctttactttgcccagggacttcctccccatgtgaacatggggaacttcgggccctgcctggagtccttgaccgctctctgtgggccccacccactctgtcctgggaaatgaagaagcatcttccttaggtctgccctgcttgcaaatccactagcaccgaccccaccacctggttccggctctgcacgctttggggtgtggatgtcgagaggcaccacggcctcacccaggcatctgctttactctggaccataggaaacaagaccgtttggaggtttcatcaggattttgggtttttcacatttcacgctaaggagtagtggccctgacttccggtcggctggccagctgactccctagggccttcagacgtgtatgcaaatgagtgatggataaggatgagtcttggagttgcgggcagcctggagactcgtggacttaccgcctggaggcaggcccgggaaggctgctgtttactcatcgggcagccacgtgctctctggaggaagtgatagtttctgaaaccgctcagatgttttggggaaagttggagaagccgtggccttgcgagaggtggttacaccagaacctggacattggccagaagaagcttaagtgggcagacactgtttgcccagtgtttgtgcaaggatggagtgggtgtctctgcatcacccacagccgcagctgtaaggcacgctggaaggcacacgcctgccaggcagggcagtctggcgcccatgatgggagggattgacatgtttcaacaaaataatgcacttccttacctagtggcccttcacacaacttttgaatctctaaaaatccataaaatccttaaagaactgtaa

SEQ ID NO: 116 - DNA, 1788 bp, Homo sapiens
atgcgcgtcctcctcgccgcgctgggactgctgttcctgggggcgctacgagccttcccacaggatcgacccttcgaggacacctgtcatggaaaccccagccactactatgacaaggctgtcaggaggtgctgttaccgctgccccatggggctgttcccgacacagcagtgcccacagaggcctactgactgcaggaagcagtgtgagcctgactactacctggatgaggccgaccgctgtacagcctgcgtgacttgttctcgagacgacctcgtggagaagacgccgtgtgcatggaactcctcccgtgtctgcgaatgtcgacccggcatgttctgttccacgtctgccgtcaactcctgtgcccgctgcttcttccattctgtctgtccggcagggatgattgtcaagttcccaggcacggcgcagaagaacacggtctgtgagccggcttccccaggggtcagccctgcctgtgccagcccagagaactgcaaggaaccctccagtggcaccatcccccaggccaagcccaccccggtgtccccagcaacctccagtgccagcaccatgcctgtaagagggggcacccgcctcgcccaggaagctgcttctaaactgacgagggctcccgactctccctcctctgtgggaaggcctagttcagatccaggtctgtccccaacacagccatgcccagaggggtctggtgattgcagaaagcagtgtgagcccgactactacctggacgaggccggccgctgcacggcctgcgtgagctgttctcgagatgaccttgtggagaagacgccatgtgcatggaactcctcccgcacctgcgaatgtcgacctggcatgatctgtgccacatcagccaccaactcccgtgcccgctgtgtcccctacccaatctgtgcagcagagacggtcaccaagccccaggatatggctgagaaggacaccacctttgaggcgccacccctggggacccagccggactgcaaccccaccccagagaatggcgaggcgcctgccagcaccagccccactcagagcttgctggtggactcccaggccagtaagacgctgcccatcccaaccagcgctcccgtcgctctctcctccacggggaagcccgttctggatgcagggccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagtga

SEQ ID NO: 117 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSRARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 118 - DNA, 2573 bp, Homo sapiens
gaatcggccgatgtgaaccgaatgttgatgtaagaggcagggcactcggctgcggatgggtaacagggcgtgggctggcacacttacttgcaccagtgcccagagagggggtgcaggctgaggagctgcccagagcaccgctcacactcccagagtacctgaagtcggcatttcaatgacaggtgacaagggtccccaaaggctaagcgggtccagctatggttccatctccagcccgaccagcccgaccagcccagggccacggcaagcacctcccagagagacctacctgagtgagaagatccccatcccagacacaaaaccgggcaccttcagcctgcggaagctatgggccttcacggggcctggcttcctcatgagcattgctttcctggacccaggaaacatcgagtcagatcttcaggctggcgccgtggcgggattcaaacttctctgggtgctgctctgggccaccgtgttgggcttgctctgccagcgactggctgcacgtctgggcgtggtgacaggcaaggacttgggcgaggtctgccatctctactaccctaaggtgccccgcaccgtcctctggctgaccatcgagctagccattgtgggctccgacatgcaggaagtcatcggcacggccattgcattcaatctgctctcagctggacgaatcccactctggggtggcgtcctcatcaccatcgtggacaccttcttcttcctcttcctcgataactacgggctgcggaagctggaagctttttttggactccttataaccattatggccttgacctttggctatgagtatgtggtggcgcgtcctgagcagggagcgcttcttcggggcctgttcctgccctcgtgcccgggctgcggccaccccgagctgctgcaggcggtgggcattgttggcgccatcatcatgccccacaacatctacctgcactcggccctggtcaagtctcgagagatagaccgggcccgccgagcggacatcagagaagccaacatgtacttcctgattgaggccaccatcgccctgtccgtctcctttatcatcaacctctttgtcatggctgtctttgggcaggccttctaccagaaaaccaaccaggctgcgttcaacatctgtgccaacagcagcctccacgactacgccaagatcttccccatgaacaacgccaccgtggccgtggacatttaccaggggggcgtgatcctgggctgcctgttcggccccgcggccctctacatctgggccataggtctcctggcggctgggcagagctccaccatgacgggcacctacgcgggacagttcgtgatggagggcttcctgaggctgcggtggtcacgcttcgcccgtgtcctcctcacccgctcctgcgccatcctgcccaccgtgctcgtggctgtcttccgggacctgagggacttgtcgggcctcaatgatctgctcaacgtgctgcagagcctgctgctcccgttcgccgtgctgcccatcctcacgttcaccagcatgcccaccctcatgcaggagtttgccaatggcctgctgaacaaggtcgtcacctcttccatcatggtgctagtctgcgccatcaacctctacttcgtggtcagctatctgcccagcctgccccaccctgcctacttcggccttgcagccttgctggccgcagcctacctgggcctcagcacctacctggtctggacctgttgccttgcccacggagccacctttctggcccacagctcccaccaccacttcctgtatgggctccttgaagaggaccagaaaggggagacctctggctaggcccacaccagggcctggctgggagtggcatgtatgacgtgactggcctgctggatgtggagggggcgcgtgcaggcagcaggatggagtgggacagttcctgagaccagccaacctgggggctttagggacctgctgtttcctagcgcagccatgtgattaccctctgggtctcagtgtcctcatctgtaaaatggagacgccaccacccttgccatggaggttaagcactttaacacagtgtctggcacttgggacaaaaacaaacaaacaaacaaaaaacatttcaaaaggtatttattgagcacctgcaggcgtgacctgacagcccaagggtgggtggggtgagggcttgaggacttgggcgggacacaggctccaaactggagcttgaaatagtgtctgatgaatgttaaattatctatctatctatttatttatttatttgagacagggaaagggtctccctctgttgccaaggctggagtgcagtggcgcaatcttaactcattgcaacctccaccttctgggttcaagcgattctctttattcagccccgggagtggcgcgcgccaccacgcccagctaatttgtgtattttcagcagagacggggtttgccatgctggccaggctggtctcgaactgctggattcaagtgatccgcccatctccgtctcccaaagtgctgggaattacaggcgtgagccaccaaaacccggcctgattaaagttaaataaatacg

SEQ ID NO: 119 - DNA, 1653 bp, Homo sapiens
atgacaggtgacaagggtccccaaaggctaagcgggtccagctatggttccatctccagcccgaccagcccgaccagcccagggccacggcaagcacctcccagagagacctacctgagtgagaagatccccatcccagacacaaaaccgggcaccttcagcctgcggaagctatgggccttcacggggcctggcttcctcatgagcattgctttcctggacccaggaaacatcgagtcagatcttcaggctggcgccgtggcgggattcaaacttctctgggtgctgctctgggccaccgtgttgggcttgctctgccagcgactggctgcacgtctgggcgtggtgacaggcaaggacttgggcgaggtctgccatctctactaccctaaggtgccccgcaccgtcctctggctgaccatcgagctagccattgtgggctccgacatgcaggaagtcatcggcacggccattgcattcaatctgctctcagctggacgaatcccactctggggtggcgtcctcatcaccatcgtggacaccttcttcttcctcttcctcgataactacgggctgcggaagctggaagctttttttggactccttataaccattatggccttgacctttggctatgagtatgtggtggcgcgtcctgagcagggagcgcttcttcggggcctgttcctgccctcgtgcccgggctgcggccaccccgagctgctgcaggcggtgggcattgttggcgccatcatcatgccccacaacatctacctgcactcggccctggtcaagtctcgagagatagaccgggcccgccgagcggacatcagagaagccaacatgtacttcctgattgaggccaccatcgccctgtccgtctcctttatcatcaacctctttgtcatggctgtctttgggcaggccttctaccagaaaaccaaccaggctgcgttcaacatctgtgccaacagcagcctccacgactacgccaagatcttccccatgaacaacgccaccgtggccgtggacatttaccaggggggcgtgatcctgggctgcctgttcggccccgcggccctctacatctgggccataggtctcctggcggctgggcagagctccaccatgacgggcacctacgcgggacagttcgtgatggagggcttcctgaggctgcggtggtcacgcttcgcccgtgtcctcctcacccgctcctgcgccatcctgcccaccgtgctcgtggctgtcttccgggacctgagggacttgtcgggcctcaatgatctgctcaacgtgctgcagagcctgctgctcccgttcgccgtgctgcccatcctcacgttcaccagcatgcccaccctcatgcaggagtttgccaatggcctgctgaacaaggtcgtcacctcttccatcatggtgctagtctgcgccatcaacctctacttcgtggtcagctatctgcccagcctgccccaccctgcctacttcggccttgcagccttgctggccgcagcctacctgggcctcagcacctacctggtctggacctgttgccttgcccacggagccacctttctggcccacagctcccaccaccacttcctgtatgggctccttgaagaggaccagaaaggggagacctctggctag

SEQ ID NO: 120 - protein, 550 aa, Homo sapiens
MTGDKGPQRLSGSSYGSISSPTSPTSPGPRQAPPRETYLSEKIPIPDTKPGTFSLRKLWAFTGPGFLMSIAFLDPGNIESDLQAGAVAGFKLLWVLLWATVLGLLCQRLAARLGVVTGKDLGEVCHLYYPKVPRTVLWLTIELAIVGSDMQEVIGTAIAFNLLSAGRIPLWGGVLITIVDTFFFLFLDNYGLRKLEAFFGLLITIMALTFGYEYVVARPEQGALLRGLFLPSCPGCGHPELLQAVGIVGAIIMPHNIYLHSALVKSREIDRARRADIREANMYFLIEATIALSVSFIINLFVMAVFGQAFYQKTNQAAFNICANSSLHDYAKIFPMNNATVAVDIYQGGVILGCLFGPAALYIWAIGLLAAGQSSTMTGTYAGQFVMEGFLRLRWSRFARVLLTRSCAILPTVLVAVFRDLRDLSGLNDLLNVLQSLLLPFAVLPILTFTSMPTLMQEFANGLLNKVVTSSIMVLVCAINLYFVVSYLPSLPHPAYFGLAALLAAAYLGLSTYLVWTCCLAHGATFLAHSSHHHFLYGLLEEDQKGETSG

(19) United States
(12) Patent Application Publication
(10) Pub. No.: US 2009/0136591 A1
(43) Pub. Date: May 28, 2009

(54) METALLOTHIONEIN AS AN EARLY BIOMARKER FOR DEATH SECONDARY TO SEPTIC SHOCK AND AS A NOVEL THERAPEUTIC TARGET FOR SEPTIC SHOCK

(76) Inventors: Hector R. Wong, Cincinnati, OH (US); Bruce J. Aronow, Cincinnati, OH (US); Thomas P. Shanley, Ann Arbor, MI (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/912,091

(22) PCT Filed: Apr. 19, 2006
(86) PCT No.: PCT/US06/14800

§ 371 Date: Jul. 21, 2008

Related U.S. Application Data
(60) Provisional application No. 60/673,656, filed on Apr. 20, 2005.

(51) Int. Cl. A61K 33/30; C12Q 1/68; A61K 31/315

(52) U.S. Cl. 424/641; 435/6; 514/494; 424/643

(57) ABSTRACT

A set of signature genes that predict the severity of septic shock, as well as methods of diagnosing and treating septic shock. The genes and methods are particularly useful for the identification of individuals who are at a high risk of death from septic shock.

### BACKGROUND OF THE INVENTION

### Field of the Invention

The present invention relates to the field of septic shock identification and treatment, particularly in individuals who are at high risk of death from septic shock.

### BACKGROUND

Septic shock is a serious condition that often occurs when an overwhelming infection leads to low blood pressure and low blood flow. If the condition is untreated, septic shock can lead to failure of vital body organs, such as the liver, heart, kidneys, and brain. Septic shock can be caused by microbial organisms, such as bacteria, fungi, or viruses. Toxins that are released by the infecting organism can cause low blood pressure, tissue damage, and loss of organ function.

The condition can occur in individuals of any age, but is usually found in elderly individuals and in children. Septic shock is particularly problematic in pediatric patients.

Symptoms of septic shock can vary but include, for example, palpitations, lightheadedness, presence of a high or very low temperature, shortness of breath, chills, agitation, confusion, rapid heart rate, and low blood pressure.

Several factors can increase the risk of septic shock. For example, septic shock risk increases with the presence of an underlying illness, such as a genitourinary tract disease, a biliary system disease, an intestinal disease, diabetes, hematologic cancers such as lymphoma or leukemia, cancer, heart disease, immunological disease, lung disease, or infection. Septic shock can also occur in normal individuals that have no additional underlying diseases or conditions.

Current treatments involve providing oxygen, supporting poorly functioning organs, administration of antibiotics, and administration of intravenous fluids.

### SUMMARY OF THE INVENTION

The invention relates to a set of signature genes that predict the severity of septic shock, as well as methods of diagnosing and treating septic shock. The genes and methods are particularly useful for the identification of individuals who are at a high risk of death from septic shock.

In some embodiments of the present invention, an assay to determine the potential of high risk septic shock in an individual is provided, by obtaining a biological sample from the individual, and determining a level of expression of at least one septic shock signature gene, where an increased level of expression of the at least one septic shock signature gene indicates an elevated risk of death from septic shock. The signature gene can encode, for example, a metallothionein protein, Metallothionein 1E, Metallothionein 1F, Metallothionein 1G, Metallothionein 1H, Metallothionein 1K, Metallothionein 1X, Granzyme B (cytotoxic serine protease), Dual specific phosphatase 2 (inactivation of MAPK), Regulator of G-protein signaling 1, v-Jun, Jun dimerization protein, Chemokine ligand 2 (MCP-1), Chemokine ligand 3 (MIP-1α), Chemokine (C—C motif) receptor-like 2, cAMP responsive element modulator, Complement factor H, SOCS1, Interferon-γ, or Interferon regulatory factor 7. The individual can be a mammal. The mammal can be, for example, a human. The human can be, for example, an elderly person, an adult, a child, an infant, a newborn, or an unborn child. The sample can be, for example, a blood sample, a tissue sample, an amniotic fluid sample, a urine sample, or a bronchoalveolar lavage sample.

In additional embodiments of the present invention, a test kit for the early identification of high risk septic shock is provided, using two or more nucleic acid sequences adapted for indicating presence or absence of at least one septic shock signature gene in a biological sample. The kit can have, for example, a probe that determines the presence of metallothionein mRNA or protein in a sample. The kit can also contain at least one of the following components: an instruction sheet, a sample collection device, a sample preparation device, positive controls, and negative controls.

In additional embodiments of the present invention, a method of treating an individual having septic shock is provided, by administering a metallothionein-reducing agent.

In further embodiments of the present invention, a method of treating an individual having septic shock is provided, by administering an agent that downregulates at least one of the genes listed in tables 2 or 3.

In a yet further embodiment of the present invention, a method of treating septic shock in an individual is provided, by administering an agent that upregulates at least one of the genes listed in table 4.

In a yet further embodiment of the present invention, a method of treating septic shock in an individual is provided, by administering zinc. The zinc can be, for example, in at least one form selected from the group consisting of: zinc sulfate, zinc gluconate, and zinc chloride. The zinc can be administered intravenously.

In a yet further embodiment of the present invention, a method of identifying an individual at high risk of death from septic shock is provided, by identifying an individual that may have septic shock, obtaining a blood or other bodily sample from the individual, testing the sample for at least one of septic shock signature genes, and determining an altered signature gene profile as compared to control samples, thereby determining that an elevated risk of death from septic shock exists in the individual. In some embodiments, at least 5 septic shock signature genes are tested. The control samples can be obtained, for example, from individuals with septic shock who were able to survive the episode. The testing can be performed, for example, by microarray analysis or a dipstick assay.

### BRIEF DESCRIPTION OF THE DRAWINGS

FIG. 1 is a cluster analysis of 400 genes that are predictors of non-survivorship. The metallothionein genes are shown. The samples from the non-surviving patients are indicated. The color coding indicates the level of gene expression. Red indicates high level expression, blue indicates decreased expression, and yellow indicates no change from baseline.

FIG. 2 is a three-dimensional principle components analysis of the patients. The analysis is based on the relative expression of approximately 400 genes that are predictors of non-survivorship. The color coding indicates the individuals who were either septic shock survivors, septic shock non-survivors, systemic inflammatory response syndrome (SIRS) survivors, or SIRS resolved individuals, along with controls. All 400 genes used for the analysis had statistically significant differential expression in non-survivors compared to survivors.

FIG. 3 is a summary of the motifs of the MT gene family members. The method uses a MEME (Multiple EM for Motif Elicitation) analysis. The features of the promoters that are activated during death serve as biomarker indicators as well as mechanistic indicators of the triggers of the death response pathway. Accordingly, disabling their activation may result in a decrease in the risk of death in these patients. The induced and the un-induced MT family members are shown.

FIG. 4 is a color-coded gene expression map. Several metallothionein genes are upregulated in the non-surviving septic shock patients as compared to the septic shock survivors.

FIG. 5 is a bar graph showing the zinc levels in serum samples of the surviving and non-surviving septic shock patients.

### DETAILED DESCRIPTION OF THE PREFERRED EMBODIMENT

Septic shock often progresses to dangerous levels, particularly in elderly patients and in children, even before its presence or severity is recognized. In fact, the individuals who are at high risk for death may have no outward symptoms of the extreme severity of the situation. Diagnosis of septic shock is difficult because it is difficult to determine which individuals are likely to survive, and which individuals are at high risk of succumbing to the disease. If those individuals who are at high risk of death can be determined readily, those individuals can be given urgent, immediate, life-saving treatments. Alternatively, many of the life-saving treatments are also of high risk to the patient, so they would not be appropriate for cases of sepsis that are not emergencies. The ability to quickly stratify the patients by their risk level would be a valuable medical tool. High risk therapies could be given to the sickest patients that would derive the most benefit, thus more favorably balancing the risk-to-benefit ratio in the patients.

In response to the need for reliable biomarkers that can predict adverse outcome of septic shock in an individual, a study of pediatric patients with septic shock was undertaken. The study involved the development of a national-level data bank of children with septic shock, which includes whole blood-derived mRNA, parallel serum samples, DNA, and extensive annotated clinical data. The databank was used to conduct microarray analyses to determine the genome-level expression profiles in pediatric septic shock.

One analysis involved 13 normal children (controls) and 16 patients with septic shock (5 deaths). In this data set, children with septic shock who progressed to death demonstrated a unique genome-level signature of gene activation and gene repression. Example 1 describes the details of the patient database, while Table 4 lists the patients, their disease, survivability, and clinical results.

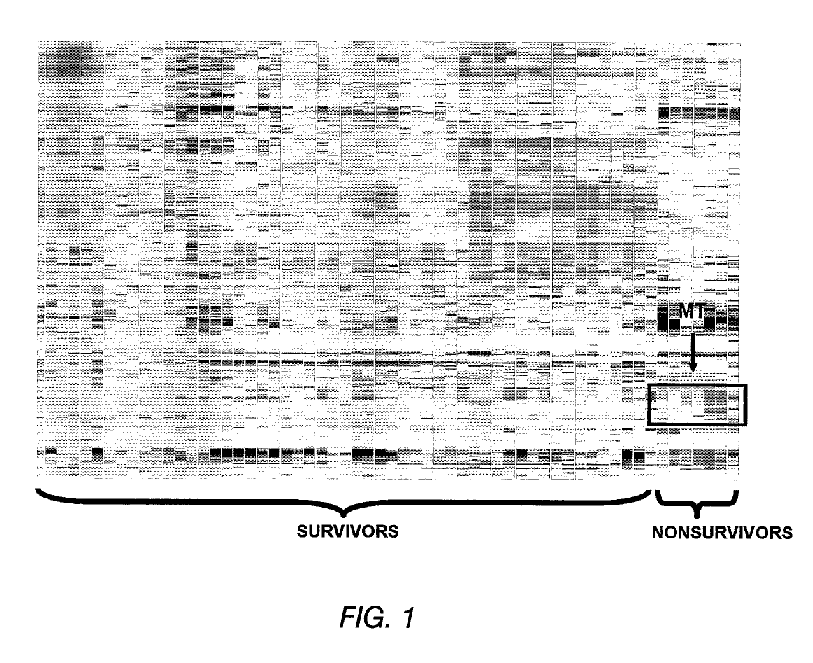

Approximately 400 signature genes have been found to be differentially regulated during septic shock. A cluster analysis of the gene expression of these 400 signature genes is shown in FIG. 1. The non-survivors exhibited a unique set of upregulated signature genes (see the outlined boxes shown in FIG. 1). Table 1, below, lists the 400 genes, their accession numbers, and related molecular and biological information. Within this set of signature genes, the metallothionein (MT) family of genes was particularly strong in predicting death of the patient. Thus, metallothionein expression can be used as a predictor of particularly high risk forms of septic shock.

These data represent 60 individual microarray chips within which there are 5 non-survivors represented by 7 individual microarray chips. We have recently analyzed an additional 63 microarray chips which include an additional 4 non-survivors represented by 7 additional microarray chips. Within this data set of 163 chips, the metallothionein signature in the non-survivors continues to be present. Specifically, metallothionein isoforms -1E, -1G, and -1M are overexpressed in the non-survivors, relative to the survivors.

TABLE 1
Septic Shock Signature Genes

| Description | Genbank | Product | GO biological process | GO molecular function | GO cellular component |
|---|---|---|---|---|---|
| ubiquinol-cytochrome c reductase core protein II | NM_003366 | ubiquinol-cytochrome c | oxidative phosphorylation; | ubiquinol-cytochrome-c | mitochondrial electron |
| | | reductase core protein II | aerobic respiration; | reductase activity; | transport chain; |
| | | | electron transport; | metalloendopeptidase | mitochondrion |
| | | | proteolysis and | activity; oxidoreductase | |
| | | | peptidolysis | activity | |
| ret finger protein 2 | NM_052811 | ret finger protein 2 | morphogenesis; negative | zinc ion binding | intracellular |
| | | | regulation of cell cycle | | |
| Homo sapiens cDNA FLJ23646 fis, | AK074226 | | | | |
| clone COL03258 | | | | | |
| Homo sapiens transcribed sequences | BG391643 | | | | |
| KIAA0460 protein | BX641025 | hypothetical protein | | 3700; transcription | |
| | | | | factor; predicted/computed; | |
| | | | | 3677; DNA binding; | |
| | | | | predicted/computed | |
| Homo sapiens cDNA FLJ10158 fis, | AK091904 | | | | |
| clone HEMBA1003463. | | | | | |
| hypothetical protein FLJ39485 | NM_175920 | hypothetical protein | proteolysis and peptidolysis | aminopeptidase activity; | integral to membrane |
| | | FLJ39485 | | metallopeptidase | |
| | | | | activity; zinc ion binding | |
| | | | | activity; membrane | |
| | | | | alanyl aminopeptidase activity | |
| Homo sapiens cDNA FLJ10673 fis, | AK024111 | | | | |
| clone NT2RP2006393. | | | | | |
| Homo sapiens cDNA FLJ10673 fis, | AK024111 | | | | |
| clone NT2RP2006393. | | | | | |
| KIAA0794 protein | AB018337 | KIAA0794 protein | | | |
| Homo sapiens transcribed | AL043343 | | | | |
| sequences | | | | | |
| KIAA2010 | NM_032560 | hypothetical protein | | | |
| | | FLJ20707 isoform 2; | | | |
| | | hypothetical protein | | | |
| | | FLJ20707 isoform 1 | | | |
| Homo sapiens mRNA; cDNA | NM_052911 | | | | |
| DKFZp313E1410 (from clone | | | | | |
| DKFZp313E1410).; KIAA1911 | | | | | |
| protein | | | | | |
| Homo sapiens transcribed | BX104926 | | | | |
| sequence with weak similarity to | | | | | |
| protein ref: NP_060265.1 | | | | | |
| (H. sapiens) hypothetical protein | | | | | |
| FLJ20378 [Homo sapiens] | | | | | |
| enhancer of polycomb homolog 1, | NM_025209 | enhancer of polycomb 1 | | | |
| (Drosophila) | | | | | |
| chromosome 20 open reading | NM_024331 | chromosome 20 open | transport | transporter activity | intracellular |
| frame 121 | | reading frame 121 | | | |
| zinc finger protein | NM_014415 | zinc finger protein ZNF- | ‘de novo’ pyrimidine base | protein binding | aspartate |
| | | U69274 | biosynthesis | | carbamoyltransferase |
| | | | | | complex |
| casein kinase 2, alpha 1 | NM_177559 | casein kinase II alpha 1 | protein amino acid | protein kinase CK2 | plasma membrane; nucleus |
| polypeptide | | subunit isoform a; | phosphorylation; signal | activity; ATP binding; | |
| | | casein kinase II alpha 1 | transduction | protein serine/threonine | |
| | | subunit isoform b | | kinase activity; | |
| | | | | transferase activity | |
| synonyms: A6, MGC23788, | NM_198974 | twinfilin isoform 1; | protein amino acid | protein-tyrosine kinase | intracellular; actin cytoskeleton |
| MGC41876; isoform 2 is encoded | | twinfilin isoform 2 | phosphorylation | activity; actin binding; | |
| by transcript variant 2; protein | | | | transferase activity | |
| tyrosine kinase 9; A6 protein | | | | | |
| tyrosine kinase | | | | | |
| DEAD (Asp-Glu-Ala-Asp) box | NM_007372 | RNA helicase-related | | | GO: 5524; DEAD; ATP |
| polypeptide 42 | | protein | | | binding; 2.1e−84; |
| | | | | | extended:inferred from |
| | | | | | electronic annotation |
| hypothetical protein FLJ10707 | AB051544 | KIAA1757 protein | | | |
| synonyms: FLJ10042, FLJ11979, | NM_020690 | FLJ20288 protein | | 3676; nucleic acid | |
| FLJ14127, KIAA1085; putative | | | | binding; | |
| protein; Homo sapiens FLJ20288 | | | | extended:Unknown; KH; | |
| protein (FLJ20288), mRNA. | | | | 1.9e−11 | |
| Homo sapiens transcribed | BX109218 | | | | |
| sequences | | | | | |
| KIAA0907 protein | NM_014949 | KIAA0907 protein | | | |
| ribosomal protein S4, X-linked | NM_001007 | ribosomal protein S4, X- | protein biosynthesis; | structural constituent of | ribosome; cytosolic small |
| | | linked X isoform | development; cell | ribosome; RNA binding | ribosomal subunit (sensu |
| | | | proliferation; regulation of cell | | Eukarya); intracellular |
| | | | cycle | | |
| Homo sapiens transcribed | BX116041 | | | | |
| sequences | | | | | |
| golgi associated PDZ and coiled- | NM_020399 | golgi associated PDZ | | protein binding | |
| coil motif containing | | and coiled-coil motif | | | |
| | | containing | | | |
| Homo sapiens transcribed | AW978341 | | | | |
| sequences | | | | | |
| Homo sapiens transcribed | AL711520 | | | | |
| sequences | | | | | |
| Homo sapiens cDNA FLJ20653 fis, | AK055922 | | | | |
| clone KAT01739 | | | | | |
| Homo sapiens transcribed | AW972041 | | | | |
| sequences | | | | | |
| NP220 nuclear protein | NM_014497 | NP220 nuclear protein | | | |
| splicing factor 3b, subunit 1, | NM_012433 | splicing factor 3b, | nuclear mRNA splicing, | pre-mRNA splicing | spliceosome complex |
| 155 kDa | | subunit 1, 155 kDa | via spliceosome | factor activity | |
| splicing factor 3b, subunit 1, | NM_012433 | splicing factor 3b, | nuclear mRNA splicing, | pre-mRNA splicing | spliceosome complex |
| 155 kDa | | subunit 1, 155 kDa | via spliceosome | factor activity | |
| myeloid/lymphoid or mixed-lineage | NM_170606 | myeloid/lymphoid or | regulation of transcription, | methyltransferase | nucleus |
| leukemia3 | | mixed-lineage leukemia 3 | DNA-dependent; | activity; DNA binding; | |
| | | | chromatin modification | histone-lysine N- | |
| | | | | methyltransferase | |
| | | | | activity | |
| Homo sapiens mRNA; cDNA | BU736292 | | | | |
| DKFZp434G0972 (from clone | | | | | |
| DKFZp434G0972) | | | | | |
| protein kinase C, beta 1 | NM_002738 | protein kinase C, beta 1 | protein amino acid | protein kinase C activity; | cytoplasm; plasma |
| | | | phosphorylation; | ATP binding; calcium ion | membrane |
| | | | intracellular signaling | binding; transferase | |
| | | | cascade | activity; diacylglycerol | |
| | | | | binding | |
| ROD1 regulator of differentiation 1 | NM_005156 | ROD1 regulator of | embryogenesis and | RNA binding activity | GO: 3723; RNA binding; |
| (S. pombe) | | differentiation 1 | morphogenesis | | predicted/computed |
| hypothetical protein FLJ13456 | AB051517 | KIAA1730 protein | | | |
| vav-1 interacting Kruppel-like | NM_138494 | vav-1 interacting | | | GO: 3676; KRAB; nucleic |
| protein | | Kruppel-like protein | | | acid binding; 7.6e−27; |
| | | isoform b; vav-1 | | | extended:inferred from |
| | | interacting Kruppel-like | | | electronic annotation |
| | | protein isoform a | | | |
| HECT domain containing 1 | NM_015382 | HECT domain | ubiquitin cycle | ubiquitin-protein ligase | intracellular |
| | | containing 1 | | activity; receptor activity | |
| nuclear receptor coactivator 2 | NM_006540 | nuclear receptor | regulation of transcription, | transcription co-activator | nucleus |
| | | coactivator 2 | DNA-dependent; signal | activity; signal | |
| | | | transduction | transducer activity | |
| Homo sapiens hypothetical protein | NM_173569 | hypothetical protein | | | |
| FLJ25778 (FLJ25778), mRNA. | | FLJ25778 | | | |
| PR domain containing 2, with ZNF | NM_012231 | retinoblastoma protein- | regulation of transcription, | zinc ion binding; | nucleus |
| domain | | binding zinc finger | DNA-dependent | transcription factor | |
| | | protein isoform a; | | activity | |
| | | retinoblastoma protein- | | | |
| | | binding zinc finger | | | |
| | | protein isoform b | | | |
| synonym: KIAA0183; alternatively | NM_014612 | C9orf10 protein | | | |
| spliced; Homo sapiens | | | | | |
| chromosome 9 open reading frame | | | | | |
| 10 (C9orf10), mRNA. | | | | | |
| hypothetical protein FLJ10246 | NM_018038 | hypothetical protein | | | |
| | | FLJ10246 | | | |
| WD repeat domain 30 | NM_030803 | APG16 autophagy 16- | | | |
| | | like isoform 2; APG16 | | | |
| | | autophagy 16-like | | | |
| | | isoform 1; APG16 | | | |
| | | autophagy 16-like | | | |
| | | isoform 3 | | | |
| Homo sapiens, clone | BC035091 | | | | |
| IMAGE: 4814008, mRNA | | | | | |
| hypothetical protein FLJ10803 | NM_018224 | hypothetical protein | | | |
| | | FLJ10803 | | | |
| PRO0471 protein | AF111846 | PRO0471 | | | |
| Homo sapiens transcribed | AA744471 | | | | |
| sequences | | | | | |
| protein kinase, lysine deficient 1 | NM_018979 | protein kinase, lysine | | | |
| | | deficient 1 | | | |
| MAD, mothers against | NM_005359 | MAD, mothers against | SMAD protein | transcription cofactor | cytoplasm; nucleus |
| decapentaplegic homolog 4 | | decapentaplegic | heteromerization; | activity; transcription | |
| (Drosophila) | | homolog 4 | regulation of transcription, | factor activity | |
| | | | DNA-dependent | | |
| Homo sapiens cDNA FLJ33199 fis, | AK090518 | | | | |
| clone ADRGL2006377. | | | | | |
| KIAA1935 protein | AK055921 | | | | |
| Homo sapiens transcribed | BG566236 | | | | |
| sequences | | | | | |
| 6-phosphogluconolactonase | NM_012088 | 6- | pentose-phosphate shunt; | hydrolase activity; 6- | GO: 17057; 6- |
| | | phosphogluconolactonase | carbohydrate metabolism | phosphogluconolactonase | phosphogluconolactonase |
| | | | | activity | activity; inferred from |
| | | | | | electronic annotation |
| | | | | | GO: 16787; hydrolase |
| | | | | | activity; inferred from |
| | | | | | electronic annotation |
| activating transcription factor 6 | NM_007348 | activating transcription | unfolded protein response, | RNA polymerase II | perinuclear space; nuclear |
| | | factor 6 | target gene transcriptional | transcription factor | membrane; nucleoplasm; |
| | | | activation; protein folding; | activity; transcription co- | endoplasmic reticulum |
| | | | signal transduction; | activator activity | membrane; integral to |
| | | | regulation of transcription | | membrane |
| | | | from Pol II promoter | | |
| Wilms tumor 1 associated protein | NM_004906 | Wilms' tumor 1- | | | |
| | | associating protein | | | |
| | | isoform 1; Wilms' tumor | | | |
| | | 1-associating protein | | | |
| | | isoform 2 | | | |
| Homo sapiens mRNA; cDNA | AL832319 | hypothetical protein | | | |
| DKFZp547A2015 (from clone | | | | | |
| DKFZp547A2015); complete cds | | | | | |
| Homo sapiens cDNA clone | AK096401 | | | | |
| IMAGE: 6653606, partial cds | | | | | |
| synonyms: FLJ10215, FLJ11824, | NM_025185 | putative ankyrin-repeat | | | |
| KIAA1148, KIAA1636; ORF1; | | containing protein | | | |
| Homo sapiens putative ankyrin- | | | | | |
| repeat containing protein | | | | | |
| (DKFZP564D166), mRNA. | | | | | |
| enhancer of zeste homolog 1 | NM_001991 | enhancer of zeste | morphogenesis; regulation | chromatin binding | nucleus |
| (Drosophila) | | homolog 1 | of transcription, DNA- | | |
| | | | dependent | | |
| Homo sapiens transcribed | BX110944 | | | | |
| sequences | | | | | |
| ADP-ribosylation factor domain | NM_001656 | ADP-ribosylation factor | small GTPase mediated | small monomeric | intracellular |
| protein 1, 64 kDa | | domain protein 1 | signal transduction | GTPase activity; GTP | |
| | | isoform alpha; ADP- | | binding; enzyme | |
| | | ribosylation factor | | activator activity; zinc ion | |
| | | domain protein 1 | | binding | |
| | | isoform beta; ADP- | | | |
| | | ribosylation factor | | | |
| | | domain protein 1 | | | |
| | | isoform gamma | | | |
| splicing factor, arginine/serine-rich | NM_004768 | splicing factor p54 | RNA splicing; regulation of | pre-mRNA splicing | nucleus |
| 11 | | | transcription, DNA- | factor activity; RNA | |
| | | | dependent; nuclear mRNA | binding; DNA binding | |
| | | | splicing, via spliceosome | | |
| staufen, RNA binding protein, | NM_014393 | staufen homolog 2 | | double-stranded RNA | GO: 3725; double-stranded RNA |
| homolog 2 (Drosophila) | | | | binding | |
| | | | | | binding; predicted/computed |
| nudix (nucleoside diphosphate | NM_006703 | nudix-type motif 3 | diadenosine | diphosphoinositol- | GO: 8486; diphosphoinositol |
| linked moiety X)-type motif 3 | | | polyphosphate catabolism; | polyphosphate | polyphosphate |
| | | | cell-cell signaling | diphosphatase activity; | phosphohydrolase; |
| | | | | hydrolase activity | predicted/computed |
| hypothetical protein dJ465N24.2.1 | NM_020317 | hypothetical protein | | | |
| | | dJ465N24.2.1 | | | |
| Homo sapiens cDNA FLJ13202 fis, | AK023264 | | | | |
| clone NT2RP3004503. | | | | | |
| Rho-associated, coiled-coil | NM_005406 | Rho-associated, coiled- | Rho protein signal | ATP binding; protein | intracellular |
| containing protein kinase 1 | | coil containing protein | transduction; protein | serine/threonine kinase | |
| | | kinase 1 | amino acid | activity; transferase | |
| | | | phosphorylation; | activity | |
| | | | intracellular signaling | | |
| | | | cascade; actin | | |
| | | | cytoskeleton organization | | |
| | | | and biogenesis | | |
| myelin basic protein | NM_002385 | myelin basic protein | nerve ensheathment; | DNA binding; structural | nucleus |
| | | | central nervous system | constituent of myelin | |
| | | | development; synaptic | sheath | |
| | | | transmission; regulation of | | |
| | | | transcription, DNA- | | |
| | | | dependent; immune | | |
| | | | response | | |
| Homo sapiens cDNA FLJ12232 fis, | AK022294 | | | | |
| clone MAMMA1001206. | | | | | |
| Homo sapiens transcribed | CA503163 | | | | |
| sequences | | | | | |
| Homo sapiens cDNA clone | CA430188 | | | | |
| IMAGE: 5294561, partial cds | | | | | |
| Homo sapiens cDNA clone | CA430188 | | | | |
| IMAGE: 5294561, partial cds | | | | | |
| Homo sapiens cDNA FLJ39934 fis, | AL831930 | hypothetical protein | | | |
| clone SPLEN2021458, weakly | | | | | |
| similar to Mus musculus mdgl-1 | | | | | |
| mRNA. | | | | | |
| KIAA1093 protein | XM_039385 | similar to KIAA1093 | | | |
| | | protein | | | |
| secretory carrier membrane protein 1 | NM_004866 | secretory carrier | post-Golgi transport; | protein transporter | integral to membrane; |
| | | membrane protein 1 | intracellular protein | activity | membrane fraction |
| | | isoform 1; secretory | transport | | |
| | | carrier membrane | | | |
| | | protein 1 isoform 2 | | | |
| PEST-containing nuclear protein | NM_020357 | PEST-containing | | | |
| | | nuclear protein | | | |
| splicing factor, arginine/serine-rich 6 | NM_006275 | arginine/serine-rich | mRNA splice site | pre-mRNA splicing | nucleus |
| | | splicing factor 6 | selection; regulation of | factor activity; RNA | |
| | | | transcription, DNA- | binding; DNA binding | |
| | | | dependent; nuclear mRNA | | |
| | | | splicing, via spliceosome | | |
| musashi homolog 2 (Drosophila) | NM_170721 | musashi 2 isoform a; | | | |
| | | musashi 2 isoform b | | | |
| Homo sapiens cDNA FLJ34036 fis, | BQ575161 | | | | |
| clone FCBBF2005069. | | | | | |
| Homo sapiens cDNA FLJ39245 fis, | AK096564 | | | | |
| clone OCBBF2008366. | | | | | |
| F-box only protein 9 | NM_033480 | F-box only protein 9 | | | |
| | | isoform 1; F-box only | | | |
| | | protein 9 isoform 2; F- | | | |
| | | box only protein 9 | | | |
| | | isoform 3 | | | |
| eukaryotic translation initiation | NM_012154 | eukaryotic translation | protein biosynthesis | translation initiation | cellular_component |
| factor 2C, 2 | | initiation factor 2C, 2 | | factor activity | unknown |
| hypothetical protein MGC40368 | NM_152772 | hypothetical protein | | | |
| | | MGC40368 | | | |
| SH3-domain GRB2-like endophilin | NM_020145 | SH3-containing protein | | | |
| B2 | | SH3GLB2 | | | |
| DKFZp564J157 protein | NM_018457 | DKFZp564J157 protein | mRNA metabolism | RNA binding activity; | cytoplasm; nucleus; |
| | | | | DNA binding activity | ribonucleoprotein complex |
| O-linked N-acetylglucosamine | NM_003605 | O-linked GlcNAc | response to nutrients; O- | acetylglucosaminyltransferase | cytosol; nucleus |
| (GlcNAc) transferase (UDP-N- | | transferase isoform 3; | linked glycosylation; signal | activity; protein | |
| acetylglucosamine:polypeptide-N- | | O-linked GlcNAc | transduction | binding activity; | |
| acetylglucosaminyl transferase) | | transferase isoform 1; | | transferase activity, | |
| | | O-linked GlcNAc | | transferring glycosyl | |
| | | transferase isoform 2 | | groups | |
| stannin | NM_003498 | Stannin | response to abiotic | | integral to membrane |
| | | | stimulus; response to | | |
| | | | stress | | |
| tubulin, beta 1 | NM_030773 | beta tubulin 1, class VI | microtubule-based | GTP binding; structural | microtubule |
| | | | movement | molecule activity | |
| phosphoinositide-3-kinase, | NM_005026 | phosphoinositide-3- | | 16303; | |
| catalytic, delta polypeptide | | kinase, catalytic, delta | | phosphatidylinositol 3- | |
| | | polypeptide | | kinase; | |
| | | | | extended:Unknown; | |
| | | | | PI3K_p85B; 4e−26 | |
| egl nine homolog 2 (C. elegans) | NM_017555 | EGL nine (C. elegans) | | | |
| | | homolog 2 isoform 2; | | | |
| | | EGL nine (C. elegans) | | | |
| | | homolog 2 isoform 1; | | | |
| | | EGL nine (C. elegans) | | | |
| | | homolog 2 isoform 3 | | | |
| caspase 2, apoptosis-related | NM_032982 | caspase 2 isoform 2 | apoptotic program; | caspase-2 activity | GO: 4202; caspase-2; |
| cysteine protease (neural | | precursor; caspase 2 | proteolysis and | | experimental evidence |
| precursor cell expressed, | | isoform 1 preproprotein; | peptidolysis | | |
| developmentally down-regulated 2) | | caspase 2 isoform 3; | | | |
| | | caspase 2 isoform 4 | | | |
| TPA regulated locus | NM_018475 | TPA regulated locus | | molecular_function | membrane |
| | | | | unknown | |
| Homo sapiens transcribed | AI807658 | | | | |
| sequences | | | | | |
| RAD23 homolog B (S. cerevisiae) | NM_002874 | UV excision repair | nucleotide-excision repair | single-stranded DNA | nucleus |
| | | protein RAD23 homolog B | | binding | |
| IQ motif containing GTPase | NM_003870 | IQ motif containing | small GTPase mediated | GTPase inhibitor | actin filament |
| activating protein 1 | | GTPase activating | signal transduction | activity; Ras GTPase | |
| | | protein 1 | | activator activity; | |
| | | | | calmodulin binding | |
| transducin (beta)-like 1X-linked | NM_005647 | transducin beta-like 1X | hearing; vision; signal | heterotrimeric G-protein | 157; peripheral plasma |
| | | | transduction | | membrane protein; |
| | | | | | predicted/computed |
| abhydrolase domain containing 2 | NM_007011 | alpha/beta hydrolase | biological_process | catalytic activity; | integral to membrane |
| | | domain containing | unknown | molecular_function | |
| | | protein 2 | | unknown | |
| sel-1 suppressor of lin-12-like (C. elegans) | NM_005065 | sel-1 suppressor of lin- | | | |
| | | 12-like | | | |
| Homo sapiens transcribed | BU899259 | | | | |
| sequences | | | | | |
| protein phosphatase 1, regulatory | NM_006242 | protein phosphatase 1, | glycogen metabolism | protein phosphatase | GO: 163; protein |
| subunit 3D | | regulatory subunit 3D | | type 1 activity; hydrolase | phosphatase type 1; |
| | | | | activity | predicted/computed |
| trichorhinophalangeal syndrome I | NM_014112 | zinc finger transcription | regulation of transcription, | transcription factor | nucleus |
| | | factor TRPS1 | DNA-dependent | activity | |
| cysteine sulfinic acid | NM_015989 | cysteine sulfinic acid | | | GO: 4782; 4.1.1.29; |
| decarboxylase | | decarboxylase-related | | | sulfinoalanine decarboxylase |
| | | protein 2 | | | activity; 4.97e−161; |
| | | | | | extended:inferred from |
| | | | | | mutant phenotype |
| | | | | | GO: 16831; pyridoxal_deC; |
| | | | | | carboxy-lyase activity; 4.5e−122; |
| | | | | | extended:Unknown |
| Cas-Br-M (murine) ecotropic | NM_005188 | Cas-Br-M (murine) | cell growth and/or | signal transducer | nucleus |
| retroviral transforming sequence | | ecotropic retroviral | maintenance; cell surface | activity; transcription | |
| | | transforming sequence | receptor linked signal | factor activity; ligase | |
| | | | transduction | activity | |
| ubiquitin-conjugating enzyme E2B | NM_003337 | ubiquitin-conjugating | postreplication repair; | ubiquitin conjugating | nucleus |
| (RAD6 homolog) | | enzyme E2B | ubiquitin cycle; ubiquitin- | enzyme activity; | |
| | | | dependent protein | ubiquitin-protein ligase | |
| | | | catabolism | activity | |
| farnesyltransferase, CAAX box, | NM_002028 | farnesyltransferase, | protein amino acid | protein | cytoplasm |
| beta | | CAAX box, beta | farnesylation | farnesyltransferase | |
| | | | | activity; | |
| | | | | prenyltransferase activity | |
| chromosome 6 open reading frame | NM_152734 | hypothetical protein | | | |
| 89 | | FLJ25357 | | | |
| Homo sapiens cDNA: FLJ21037 | AK024690 | | | | |
| fis, clone CAE10055 | | | | | |
| CDC-like kinase 4 | NM_020666 | protein serine threonine | protein amino acid | protein-tyrosine kinase | nucleus |
| | | kinase Clk4 | phosphorylation | activity; ATP binding; | |
| | | | | protein serine/threonine | |
| | | | | kinase activity; | |
| | | | | transferase activity | |
| protein kinase C-like 2 | NM_006256 | protein kinase C-like 2 | protein amino acid | ATP binding; protein | intracellular |
| | | | phosphorylation; signal | serine/threonine kinase | |
| | | | transduction | activity; transferase | |
| | | | | activity | |
| Homo sapiens mRNA activated in | AJ012498 | | | | |
| tumor suppression, clone TSAP18. | | | | | |
| ubiquitin protein ligase | NM_183414 | ubiquitin protein ligase | | | |
| | | isoform a; ubiquitin | | | |
| | | protein ligase isoform b | | | |
| Homo sapiens cDNA FLJ14111 fis, | AK024173 | | | | |
| clone MAMMA1001630. | | | | | |
| Homo sapiens transcribed | AI382001 | | | | |
| sequences | | | | | |
| striatin, calmodulin binding protein | NM_003162 | striatin, calmodulin | biological_process | calmodulin binding | cellular_component |
| | | binding protein | unknown | | unknown |
| choline phosphotransferase 1 | NM_020244 | choline | phospholipid biosynthesis; | oxidoreductase activity; | membrane |
| | | phosphotransferase 1 | electron transport | transferase activity | |
| Homo sapiens cDNA clone | AK125406 | | | | |
| IMAGE: 5223469, partial cds | | | | | |
| Homo sapiens cDNA FLJ26692 fis, | AK130202 | | | | |
| clone MPG07890 | | | | | |
| Homo sapiens cDNA FLJ30303 fis, | AK054865 | | | | |
| clone BRACE2003269. | | | | | |
| Homo sapiens transcribed | AL532522 | | | | |
| sequences | | | | | |
| coagulation factor V (proaccelerin, | NM_000130 | coagulation factor V | blood coagulation; cell | blood coagulation factor | GO: 3801; blood coagulation |
| labile factor) | | precursor | adhesion | activity; copper ion | factor; experimental |
| | | | | binding | evidence |
| Homo sapiens cDNA: FLJ21377 | AK025030 | | | | |
| fis, clone COL03255. | | | | | |
| hypothetical protein | NM_152588 | hypothetical protein | | | |
| DKFZp762A217 | | DKFZp762A217 | | | |
| Homo sapiens transcribed | BX114932 | | | | |
| sequences | | | | | |
| Homo sapiens transcribed | BG570010 | | | | |
| sequence with moderate similarity | | | | | |
| to protein sp: P39194 (H. sapiens) | | | | | |
| ALU7_HUMAN Alu subfamily SQ | | | | | |
| sequence contamination warning | | | | | |
| entry | | | | | |
| Homo sapiens transcribed | BX112864 | | | | |
| sequence with weak similarity to | | | | | |
| protein ref: NP_060190.1 | | | | | |
| (H. sapiens) hypothetical protein | | | | | |
| FLJ20234 [Homo sapiens] | | | | | |
| C-type (calcium dependent, | NM_197953 | C-type lectin, | | | |
| carbohydrate-recognition domain) | | superfamily member 12 | | | |
| lectin, superfamily member 12 | | isoforms a-i | | | |
| hemochromatosis | NM_000410 | hemochromatosis | iron ion homeostasis; | | integral to plasma |
| | | protein isoforms 1-10 | receptor mediated | | membrane; cytoplasm |
| | | | endocytosis; iron ion | | |
| | | | transport; protein complex | | |
| | | | assembly | | |
| Homo sapiens cDNA FLJ41675 fis, | AK123669 | | | | |
| clone HCASM2002148 | | | | | |
| hypothetical protein FLJ10998 | NM_018294 | hypothetical protein | | | |
| | | FLJ10998 | | | |
| caspase 2, apoptosis-related | NM_032982 | caspase 2 isoform 2 | apoptotic program; | caspase-2 activity | GO: 4202; caspase-2; |
| cysteine protease (neural | | precursor; caspase 2 | proteolysis and | | experimental evidence |
| precursor cell expressed, | | isoform 1 preproprotein; | peptidolysis | | |
| developmentally down-regulated 2) | | caspase 2 isoform 3; | | | |
| | | caspase 2 isoform 4 | | | |
| Mdm4, transformed 3T3 cell | NM_002393 | mouse double minute 4 | negative regulation of cell | 5515; protein binding; | nucleus |
| double minute 4, p53 binding | | homolog | proliferation | extended:inferred from | |
| protein (mouse) | | | | electronic annotation; | |
| | | | | MDM2; 9.5e−51 | |
| ATP-binding cassette, sub-family C | NM_000352 | ATP-binding cassette, | potassium ion transport; | sulfonylurea receptor | integral to membrane |
| (CFTR/MRP), member 8 | | sub-family C, member 8 | carbohydrate metabolism | activity; potassium ion | |
| | | | | transporter activity; | |
| | | | | nucleotide binding; ATP | |
| | | | | binding; ATP-binding | |
| | | | | cassette (ABC) | |
| | | | | transporter activity | |
| solute carrier family 30 (zinc | NM_017964 | solute carrier family 30 | | 8324; cation transporter; | |
| transporter), member 6 | | (zinc transporter), | | extended:traceable | |
| | | member 6 | | author statement; | |
| | | | | Cation_efflux; 1.4e−09 | |
| potassium voltage-gated channel, | NM_005472 | potassium voltage-gated | potassium ion transport | voltage-gated potassium | voltage-gated potassium |
| Isk-related family, member 3 | | channel, Isk-related | | channel activity | channel complex; integral to |
| | | family, member 3 | | | membrane |
| elastin microfibril interfacer 2 | NM_032048 | elastin microfibril | biological_process | protein binding activity; | extracellular |
| | | interfacer 2 | unknown | extracellular matrix | |
| | | | | constituent conferring | |
| | | | | elasticity activity | |
| solute carrier family 6 | NM_003043 | solute carrier family 6 | amino acid metabolism; | taurine:sodium | integral to plasma |
| (neurotransmitter transporter, | | (neurotransmitter | neurotransmitter transport | symporter activity | membrane |
| taurine), member 6 | | transporter, taurine), | | | |
| | | member 6 | | | |
| homeodomain interacting protein | NM_005734 | homeodomain | protein amino acid | ATP binding; protein | cellular_component |
| kinase 3 | | interacting protein | phosphorylation | serine/threonine kinase | unknown |
| | | kinase 3 | | activity; transferase | |
| | | | | activity | |
| son of sevenless (Drosophilia) | NM_006939 | son of sevenless | small GTPase mediated | guanyl-nucleotide | cellular component unknown |
| homolog 2; guanine nucleotide | | homolog 2 | signal transduction | exchange factor activity | |
| exchange factor; guanine | | | | | |
| nucleotide releasing factor; Homo | | | | | |
| sapiens son of sevenless homolog | | | | | |
| 2 (Drosophila) (SOS2), mRNA. | | | | | |
| active BCR-related gene | NM_021962 | active breakpoint cluster | small GTPase mediated | GTPase activator | GO: 5096; GTPase activator; |
| | | region-related protein | signal transduction | activity; guanyl- | experimental evidence |
| | | isoform b; active | | nucleotide exchange | |
| | | breakpoint cluster | | factor activity | |
| | | region-related protein | | | |
| | | isoform a | | | |
| peptidyl arginine deiminase, type | NM_012387 | peptidyl arginine | protein modification | protein-arginine | |
| IV | | deiminase, type IV | | deiminase activity; | |
| | | | | calcium ion binding; | |
| | | | | hydrolase activity | |
| Start codon is not identified.; Homo | XM_375926 | FLJ00095 protein | | | |
| sapiens mRNA for FLJ00095 | | | | | |
| protein.; DnaJ (Hsp40) homolog, | | | | | |
| subfamily C, member 5 | | | | | |
| flotillin 2 | NM_004475 | flotillin 2 | epidermal differentiation; | cell adhesion molecule | plasma membrane |
| | | | cell adhesion | activity | |
| alkaline phosphatase, | NM_000478 | tissue non-specific | ossification; metabolism | magnesium ion binding; | integral to membrane |
| liver/bone/kidney | | alkaline phosphatase | | alkaline phosphatase | |
| | | precursor | | activity; hydrolase | |
| | | | | activity | |
| Ras and Rab interactor 3 | NM_024832 | Ras and Rab interactor 3 | neuropeptide signaling | GTPase activator | cellular_component |
| | | | pathway; endocytosis; | activity; Ras interactor | unknown |
| | | | intracellular signaling | activity | |
| | | | cascade | | |
| chromosome 20 open reading frame 178 | NM_176812 | Snf7 homologue associated with Alix 1 | | molecular_function unknown | |
| ATPase, H+ transporting, | NM_001690 | ATPase, H+ | transport; ATP | ATP-binding and | integral to plasma |
| lysosomal 70 kDa, V1 subunit A | | transporting, lysosomal | biosynthesis; energy | phosphorylation- | membrane; cytoplasm; |
| | | 70 kD, V1 subunit A, isoform 1 | coupled proton transport, | dependent chloride | proton-transporting two- |
| | | | against the | channel activity; ATP | sector ATPase complex |
| | | | electrochemical gradient | binding; hydrolase | |
| | | | | activity; hydrogen- | |
| | | | | exporting ATPase | |
| | | | | activity, phosphorylative | |
| | | | | mechanism | |
| potassium voltage-gated channel, | NM_005472 | potassium voltage-gated | potassium ion transport | voltage-gated potassium | voltage-gated potassium |
| Isk-related family, member 3 | | channel, Isk-related | | channel activity | channel complex; integral to |
| | | family, member 3 | | | membrane |
| caspase recruitment domain | NM_021209 | caspase recruitment | apoptosis | ATP binding; apoptosis | intracellular |
| family, member 12 | | domain protein 12 | | regulator activity | |
| F11 receptor | NM_144503 | F11 receptor isoform a | cell motility; inflammatory | cell adhesion molecule | intercellular junction |
| | | precursor; F11 receptor | response | activity | |
| | | isoform b | | | |
| oxysterol binding protein-like 8 | NM_020841 | oxysterol-binding | | | |
| | | protein-like protein 8 | | | |
| pre-B-cell leukemia transcription | NM_002586 | pre-B-cell leukemia | anterior compartment | transcription factor | nucleus; ribulose |
| factor 2 | | transcription factor 2 | specification; posterior | activity; ribulose- | bisphosphate carboxylase |
| | | | compartment | bisphosphate | complex |
| | | | specification; regulation of | carboxylase activity | |
| | | | transcription, DNA- | | |
| | | | dependent; carbon | | |
| | | | utilization by fixation of | | |
| | | | carbon dioxide | | |
| myeloid/lymphoid or mixed-lineage | NM_005933 | myeloid/lymphoid or | cell growth and/or | RNA polymerase II | nucleus |
| leukemia (trithorax homolog, | | mixed-lineage leukemia | maintenance; regulation of | transcription factor | |
| Drosophila) | | (trithorax homolog, | transcription, DNA- | activity; zinc ion binding | |
| | | Drosophila) | dependent; transcription | | |
| | | | from Pol II promoter | | |
| son of sevenless (Drosophilia) | NM_006939 | son of sevenless | small GTPase mediated | guanyl-nucleotide | cellular_component |
| homolog 2; guanine nucleotide | | homolog 2 | signal transduction | exchange factor activity | unknown |
| exchange factor; guanine | | | | | |
| nucleotide releasing factor; Homo | | | | | |
| sapiens son of sevenless homolog | | | | | |
| 2 (Drosophila) (SOS2), mRNA. | | | | | |
| abhydrolase domain containing 2 | NM_007011 | alpha/beta hydrolase | biological_process | catalytic activity; | integral to membrane |
| | | domain containing | unknown | molecular_function | |
| | | protein 2 | | unknown | |
| kringle containing transmembrane | NM_032045 | kringle-containing | cell communication; | molecular_function | integral to membrane; |
| protein 1 | | transmembrane protein | biological_process | unknown | membrane fraction |
| | | 1 isoforms 1 and 2 | unknown | | |
| hypothetical protein FLJ10979 | NM_018289 | hypothetical protein | | | |
| | | FLJ10979 | | | |
| tumor differentially expressed 1 | NM_006811 | tumor differentially | | GO: 16021; integral | integral to membrane |
| | | expressed protein 1 | | membrane protein; | |
| | | | | predicted/computed | |
| tumor differentially expressed 1 | NM_006811 | tumor differentially | | GO: 16021; integral | integral to membrane |
| | | expressed protein 1 | | membrane protein; | |
| | | | | predicted/computed | |
| homeodomain interacting protein | NM_198268 | homeodomain- | | | GO: 4672; pkinase; protein |
| kinase 1 | | interacting protein | | | kinase activity; 2.7e−47; |
| | | kinase 1 isoforms 1-4 | | | extended:inferred from |
| | | | | | electronic annotation |
| hypothetical protein FLJ10613 | NM_019067 | hypothetical protein | proteolysis and | peptidase activity | membrane |
| | | FLJ10613 | peptidolysis | | |
| hypothetical protein FLJ12666 | NM_024595 | hypothetical protein | | | |
| | | FLJ12666 | | | |
| SEC14-like 1 (S. cerevisiae) | NM_003003 | SEC14 (S. cerevisiae)- | transport; nonselective | binding; transporter | membrane; Golgi apparatus; |
| | | like 1 | vesicle transport | activity | intracellular |
| MIx interactor | NM_014938 | MondoA | | | |
| coatomer protein complex, subunit | NM_004371 | coatomer protein | ER to Golgi transport; | hormone activity; protein | membrane; Golgi apparatus; |
| alpha | | complex, subunit alpha | intracellular protein | transporter activity | endoplasmic reticulum |
| | | | transport | | |
| huntingtin interacting protein B | NM_012271 | huntingtin interacting | | | |
| | | protein B isoform 2; | | | |
| | | huntingtin interacting | | | |
| | | protein B isoform 1 | | | |
| Fc fragment of IgG, low affinity IIa, | NM_021642 | Fc fragment of IgG, low | immune response | receptor activity; | integral to membrane; |
| receptor for (CD32) | | affinity IIa, receptor for | | receptor signaling | plasma membrane |
| | | (CD32) | | protein activity; IgG | |
| | | | | binding | |
| Homo sapiens cDNA FLJ14186 fis, | XM_379273 | | | | |
| clone NT2RP2005726. | | | | | |
| RAB11B, member RAS oncogene | NM_004218 | RAB11B, member RAS | small GTPase mediated | RAS small monomeric | GO: 3928; RAB small |
| family | | oncogene family | signal transduction; | GTPase activity; Rho | monomeric GTPase; |
| | | | intracellular protein | small monomeric | experimental evidence |
| | | | transport | GTPase activity; GTP | |
| | | | | binding; RAB small | |
| | | | | monomeric GTPase | |
| | | | | activity; protein | |
| | | | | transporter activity | |
| ubiquitination factor E4B (UFD2 | NM_006048 | ubiquitination factor E4B | response to UV; cell | ubiquitin conjugating | ubiquitin ligase complex; |
| homolog, yeast) | | | growth and/or | enzyme activity; | cytoplasm |
| | | | maintenance; protein | chaperone activity; | |
| | | | folding; apoptosis; protein | enzyme binding | |
| | | | ubiquitination during | | |
| | | | ubiquitin-dependent | | |
| | | | protein catabolism | | |
| tubulin, gamma complex | NM_006322 | spindle pole body protein | microtubule-based | 5198; structural | 5813; centrosome; |
| associated protein 3 | | | process | molecule; not recorded | experimental evidence; |
| | | | | | 15630; microtubule |
| | | | | | cytoskeleton; experimental |
| | | | | | evidence; 5856; |
| | | | | | cytoskeleton; not recorded |
| translocated promoter region (to | NM_003292 | translocated promoter | protein-nucleus import; | GO: 5634; nucleus; | nuclear pore; cytoplasm; |
| activated MET oncogene) | | region (to activated MET | transport | inferred from electronic | nucleus |
| | | oncogene) | | annotation GO: 5737; | |
| | | | | cytoplasm; traceable | |
| | | | | author statement | |
| | | | | GO: 5871; kinesin | |
| | | | | complex; inferred from | |
| | | | | electronic annotation | |
| | | | | GO: 5643; nuclear pore; | |
| | | | | traceable author | |
| | | | | statement | |
| hypothetical protein FLJ33215 | NM_148894 | hypothetical protein FLJ33215 | | | |
| translocated promoter region (to | NM_003292 | translocated promoter | protein-nucleus import; | | nuclear pore; cytoplasm; |
| activated MET oncogene) | | region (to activated MET | transport | | nucleus |
| | | oncogene) | | | |
| hypothetical protein MGC15606 | NM_145037 | hypothetical protein | | | |
| | | MGC15606 | | | |
| Homo sapiens mRNA; cDNA | BI857154 | | | | |
| DKFZp566E0124 (from clone | | | | | |
| DKFZp566E0124) | | | | | |
| potassium channel tetramerisation | NM_018992 | potassium channel | potassium ion transport | voltage-gated potassium | membrane; voltage-gated |
| domain containing 5 | | tetramerisation domain | | channel activity; protein | potassium channel complex |
| | | containing 5 | | binding | |
| zinc finger protein 238 | NM_006352 | zinc finger protein 238 | transport; regulation of | protein binding; DNA | nucleus |
| | | | transcription, DNA- | binding | |
| | | | dependent | | |
| retinoid X receptor, beta | NM_021976 | retinoid X receptor, beta | regulation of transcription, | retinoid-X receptor | nucleus |
| | | | DNA-dependent | activity; steroid hormone | |
| | | | | receptor activity; steroid | |
| | | | | binding; transcription co- | |
| | | | | activator activity; | |
| | | | | transcription factor | |
| | | | | activity | |
| amyloid beta (A4) precursor | NM_019043 | amyloid beta (A4) | GO: 7218; RA; | | |
| protein-binding, family B, member | | precursor protein- | neuropeptide signaling | | |
| 1 interacting protein | | binding, family B, | pathway; 0.025; | | |
| | | member 1 interacting | extended:Unknown | | |
| | | protein | | | |
| adenomatosis polyposis coli | NM_000038 | adenomatosis polyposis | cell adhesion; protein | beta-catenin binding | kinesin complex |
| | | coli | complex assembly; signal | | |
| | | | transduction; negative | | |
| | | | regulation of cell cycle | | |
| zinc finger protein 36 (KOX 18) | BX640646 | hypothetical protein | regulation of transcription, | transcription factor | nucleus |
| | | | DNA-dependent | activity | |
| tousled-like kinase 1 | NM_012290 | tousled-like kinase 1 | response to DNA damage | protein-tyrosine kinase | nucleus |
| | | | stimulus; cell cycle; | activity; ATP binding; | |
| | | | intracellular protein | protein serine/threonine | |
| | | | transport; protein amino | kinase activity; DNA | |
| | | | acid phosphorylation; | binding; transferase | |
| | | | regulation of transcription, | activity | |
| | | | DNA-dependent; | | |
| | | | intracellular signaling | | |
| | | | cascade; chromatin | | |
| | | | modification; regulation of | | |
| | | | chromatin | | |
| | | | assembly/disassembly | | |
| Homo sapiens cDNA FLJ14186 fis, | XM_379273 | growth hormone 1, | | | |
| clone NT2RP2005726. | | isoform 5 | | | |
| Homo sapiens full length insert | AF086554 | | | | |
| cDNA clone ZE14C04 | | | | | |
| solute carrier family 8 | NM_021097 | solute carrier family 8 | sodium ion transport; | sodium ion transporter | integral to plasma |
| (sodium/calcium exchanger), | | (sodium/calcium | calcium ion transport; | activity; calcium ion | membrane |
| member 1 | | exchanger), member 1 | muscle contraction | transporter activity; | |
| | | | | calmodulin binding; | |
| | | | | calcium:sodium | |
| | | | | antiporter activity | |
| chromosome 13 open reading | NM_017905 | chromosome 13 open | | | |
| frame 11 | | reading frame 11 | | | |
| amyloid beta (A4) precursor-like | NM_001642 | amyloid beta (A4) | | | 16020; membrane; |
| protein 2 | | precursor-like protein 2 | | | extended:Unknown; |
| | | | | | A4_EXTRA; 5.4e−121 |
| transketolase (Wernicke-Korsakoff | NM_001064 | transketolase | | transketolase activity; | GO: 4802; transketolase; |
| syndrome) | | | | calcium ion binding; | predicted/computed |
| | | | | transferase activity | |
| slingshot 2 | NM_033389 | slingshot 2 | | | |
| egf-like module containing, mucin- | NM_013447 | egf-like module | | | |
| like, hormone receptor-like 2 | | containing, mucin-like, | | | |
| | | hormone receptor-like | | | |
| | | sequence 2 isoforms a-g | | | |
| hypothetical protein MGC4093 | NM_030578 | hypothetical protein | | | |
| | | MGC4093 | | | |
| solute carrier family 11 (proton- | NM_000578 | solute carrier family 11 | response to bacteria; | transporter activity | integral to plasma |
| coupled divalent metal ion | | (proton-coupled divalent | response to | | membrane; membrane |
| transporters), member 1 | | metal ion transporters), | pest/pathogen/parasite; | | fraction |
| | | member 1 | transport; iron ion | | |
| | | | transport; small molecule | | |
| | | | transport | | |
| | AF229163 | | | | |
| solute carrier family 11 (proton- | NM_000578 | solute carrier family 11 | response to bacteria; | transporter activity | integral to plasma |
| coupled divalent metal ion | | (proton-coupled divalent | response to | | membrane; membrane |
| transporters), member 1 | | metal ion transporters), | pest/pathogen/parasite; | | fraction |
| | | member 1 | transport; iron ion | | |
| | | | transport; small molecule | | |
| | | | transport | | |
| N-acetylneuraminate pyruvate | NM_030769 | N-acetylneuraminate | | | |
| lyase (dihydrodipicolinate | | pyruvate lyase | | | |
| synthase) | | | | | |
| ankyrin repeat and BTB (POZ) | NM_032548 | ankyrin repeat and BTB | | 5515; protein binding; | |
| domain containing 1 | | (POZ) domain | | extended:inferred from | |
| | | containing 1 isoforms 1-3 | | electronic annotation; | |
| | | | | BTB; 7.1e−17; 5515; | |
| | | | | protein binding; | |
| | | | | extended:inferred from | |
| | | | | electronic annotation; | |
| | | | | BTB; 1.2e−16 | |
| ankyrin repeat and BTB (POZ) | NM_032548 | ankyrin repeat and BTB | | 5515; protein binding; | |
| domain containing 1 | | (POZ) domain | | extended:inferred from | |
| | | containing 1 isoforms 1-3; | | electronic annotation; | |
| | | ankyrin repeat | | BTB; 7.1e−17; 5515; | |
| | | | | protein binding; | |
| | | | | extended:inferred from | |
| | | | | electronic annotation; | |
| | | | | BTB; 1.2e−16 | |
| Homo sapiens cDNA FLJ14186 fis, | XM_379273 | | | | |
| clone NT2RP2005726. | | | | | |
| Homo sapiens cDNA FLJ11942 fis, | AK022004 | | | | |
| clone HEMBB1000652. | | | | | |
| alanyl (membrane) | NM_001150 | membrane alanine | proteolysis and | aminopeptidase activity; | integral to plasma |
| aminopeptidase (aminopeptidase | | aminopeptidase | peptidolysis; angiogenesis | metallopeptidase | membrane |
| N, aminopeptidase M, microsomal | | precursor | | activity; zinc ion binding; | |
| aminopeptidase, CD13, p150) | | | | receptor activity; | |
| | | | | membrane alanyl | |
| | | | | aminopeptidase activity; | |
| | | | | hydrolase activity | |
| synonym: MGC50452; go_function: | NM_173462 | papilin, proteoglycan-like | | | |
| serine protease inhibitor activity | | sulfated glycoprotein | | | |
| [goid 0004867] [evidence IEA]; | | | | | |
| Homo sapiens papilin, | | | | | |
| proteoglycan-like sulfated | | | | | |
| glycoprotein (PAPLN), mRNA. | | | | | |
| phosphorylase, glycogen; liver | NM_002863 | phosphorylase, | glycogen metabolism; | glycogen phosphorylase | |
| (Hers disease, glycogen storage | | glycogen; liver (Hers | carbohydrate metabolism | activity; transferase | |
| disease type VI) | | disease, glycogen | | activity, transferring | |
| | | storage disease type VI) | | glycosyl groups | |
| Homo sapiens cDNA FLJ45384 fis, | AK127315 | | | | |
| clone BRHIP3021987 | | | | | |
| hypothetical protein FLJ10298 | NM_018050 | hypothetical protein | | | |
| | | FLJ10298 | | | |
| Homo sapiens mRNA for | AB028949 | KIAA1026 protein | GO: 6470 protein | | GO: 8181 tumor suppressor |
| KIAA1026 protein, partial cds. | | | dephosphorylation | | (not recorded) GO: 163 |
| | | | (predicted/computed) | | protein phosphatase type 1 |
| | | | | | (predicted/computed) |
| | | | | | GO: 8598 protein |
| | | | | | phosphatase type 1 catalyst |
| | | | | | (not recorded) |
| transcript expressed during | NM_152914 | transcript expressed | | | |
| hematopoiesis 2 | | during hematopoiesis 2 | | | |
| hypothetical protein | NM_031305 | hypothetical protein | | | |
| DKFZp564B1162 | | DKFZp564B1162 | | | |
| taste receptor, type 2, member 40 | NM_176882 | taste receptor, type 2, | G-protein coupled receptor | G-protein coupled | integral to membrane |
| | | member 40 | protein signaling pathway | receptor activity | |
| Homo sapiens cDNA FLJ37694 fis, | AK095013 | | | | |
| clone BRHIP2015224. | | | | | |
| desmocollin 2 | NM_004949 | desmocollin 2 isoform | homophilic cell adhesion | calcium-dependent cell | cytoskeleton; intercellular |
| | | Dsc2b preproprotein; | | adhesion molecule | junction; integral to |
| | | desmocollin 2 isoform | | activity; calcium ion | membrane |
| | | Dsc2a preproprotein | | binding | |
| desmocollin 2 | NM_004949 | desmocollin 2 isoform | homophilic cell adhesion | calcium-dependent cell | cytoskeleton; intercellular |
| | | Dsc2b preproprotein; | | adhesion molecule | junction; integral to |
| | | desmocollin 2 isoform | | activity; calcium ion | membrane |
| | | Dsc2a preproprotein | | binding | |
| Homo sapiens full length insert | AI819863 | | | | |
| cDNA clone YI40A07 | | | | | |
| KIAA1181 protein | NM_020462 | KIAA1181 protein | | | |
| Homo sapiens transcribed | BF510602 | | | | |
| sequences | | | | | |
| trinucleotide repeat containing 5 | NM_006586 | trinucleotide repeat | | | |
| | | containing 5 | | | |
| ERO1-like (S. cerevisiae) | NM_014584 | ERO1-like | | | |
| hypothetical protein MGC45871 | NM_182705 | hypothetical protein | | | |
| | | MGC45871 | | | |
| hypothetical protein MGC45871 | NM_182705 | hypothetical protein | | | |
| | | MGC45871 | | | |
| RAB guanine nucleotide exchange | NM_014504 | RAB guanine nucleotide | | zinc ion binding; DNA | |
| factor (GEF) 1 | | exchange factor (GEF) 1 | | binding | |
| kinesin family member 3C | NM_002254 | kinesin family member | nonselective vesicle | ATP binding; motor | kinesin complex |
| | | 3C | transport | activity | |
| hypothetical protein BC016153 | NM_138788 | hypothetical protein | | | |
| | | BC016153 | | | |
| EF hand calcium binding protein 1 | NM_022351 | EF hand calcium binding | | calcium ion binding | |
| | | protein 1 | | | |
| tumor necrosis factor receptor | NM_001243 | tumor necrosis factor | negative regulation of cell | transmembrane receptor | integral to membrane |
| superfamily, member 8 | | receptor superfamily, | proliferation; signal | activity | |
| | | member 8 isoform 1 | transduction | | |
| | | precursor; tumor | | | |
| | | necrosis factor receptor | | | |
| | | superfamily, member 8 | | | |
| | | isoform 2 | | | |
| hypothetical protein | NM_173078 | slit and trk like 4 protein | | | |
| DKFZp547M2010 | | | | | |
| chondroitin sulfate proteoglycan 2 | NM_004385 | chondroitin sulfate | cell recognition; | sugar binding; | extracellular matrix |
| (versican) | | proteoglycan 2 | development; heterophilic | hyaluronic acid binding; | |
| | | (versican) | cell adhesion | calcium ion binding | |
| ribonuclease, RNase A family, 4 | NM_194430 | ribonuclease, RNase A | mRNA cleavage | pancreatic ribonuclease | cellular_component |
| | | family, 4 precursor | | activity; nucleic acid | unknown |
| | | | | binding; endonuclease | |
| | | | | activity; hydrolase | |
| | | | | activity | |
| Homo sapiens transcribed | BM994473 | | | | |
| sequence with weak similarity to | | | | | |
| protein ref: NP_006620.1 | | | | | |
| (H. sapiens) zinc finger protein 271 | | | | | |
| [Homo sapiens] | | | | | |
| hypothetical protein | NM_016613 | hypothetical protein | | | |
| DKFZp434L142 | | DKFZp434L142 | | | |
| chemokine (C-C motif) receptor 2 | NM_000647 | chemokine (C-C motif) | negative regulation of | C-C chemokine receptor | soluble fraction; integral to |
| | | receptor 2 isoform A; | adenylate cyclase activity; | activity; rhodopsin-like | plasma membrane |
| | | chemokine (C-C motif) | cytosolic calcium ion | receptor activity | |
| | | receptor 2 isoform B | concentration elevation; | | |
| | | | JAK-STAT cascade; G- | | |
| | | | protein coupled receptor | | |
| | | | protein signaling pathway; | | |
| | | | chemotaxis; cellular | | |
| | | | defense response; | | |
| | | | invasive growth; | | |
| | | | inflammatory response; | | |
| | | | antimicrobial humoral | | |
| | | | response (sensu | | |
| | | | Vertebrata) | | |
| CGI-90 protein | NM_016033 | CGI-90 protein | ubiquitin cycle; protein | ubiquitin-protein ligase | intracellular |
| | | | modification | activity | |
| Homo sapiens cDNA FLJ30798 fis, | BE044068 | | | | |
| clone FEBRA2001161. | | | | | |
| Homo sapiens transcribed | AV648418 | | | | |
| sequence with moderate similarity | | | | | |
| to protein pir: T02670 (H. sapiens) | | | | | |
| T02670 probable thromboxane A2 | | | | | |
| receptor isoform beta - human | | | | | |
| tumor-associated calcium signal | NM_002353 | tumor-associated | vision; cell surface | receptor activity | cytosol; integral to plasma |
| transducer 2 | | calcium signal | receptor linked signal | | membrane |
| | | transducer 2 precursor | transduction; cell | | |
| | | | proliferation | | |
| homeo box A9 | NM_152739 | homeobox protein A9 | development; oncogenesis | 3700; transcription | |
| | | isoform b; homeobox | | factor; extended:inferred | |
| | | protein A9 isoform a | | from electronic | |
| | | | | annotation; homeobox; | |
| | | | | 4.5e−30; 3700; | |
| | | | | transcription factor; | |
| | | | | extended:inferred from | |
| | | | | electronic annotation; | |
| | | | | homeobox; 7.7e−28 | |
| Homo sapiens transcribed | AW976321 | | | | |
| sequence with weak similarity to | | | | | |
| protein ref: NP_060190.1 | | | | | |
| (H. sapiens) hypothetical protein | | | | | |
| FLJ20234 [Homo sapiens] | | | | | |
| Homo sapiens mRNA; cDNA | AL117464 | | | | |
| DKFZp586I2322 (from clone | | | | | |
| DKFZp586I2322) | | | | | |
| KIAA1036 | NM_014909 | KIAA1036 | | | |
| Homo sapiens cDNA FLJ30761 fis, | BC035116 | | | | |
| clone FEBRA2000538. | | | | | |
| palladin | NM_016081 | palladin | amino acid metabolism | | |
| thymic stromal co-transporter | NM_033051 | thymic stromal co- | | | |
| | | transporter | | | |
| carboxypeptidase, vitellogenic-like | NM_019029 | serine carboxypeptidase | proteolysis and | serine carboxypeptidase | |
| | | vitellogenic-like | peptidolysis | activity; hydrolase | |
| | | | | activity | |
| UI-H-FL1-bfx-k-20-0-UI.s1 | BU620670 | | | | |
| NCI_CGAP_FL1 Homo sapiens | | | | | |
| cDNA clone UI-H-FL1-bfx-k-20-0- | | | | | |
| UI 3′, mRNA sequence. | | | | | |
| chemokine (C-C motif) receptor 2 | NM_000647 | chemokine (C-C motif) | negative regulation of | C-C chemokine receptor | soluble fraction; integral to |
| | | receptor 2 isoform A; | adenylate cyclase activity; | activity; rhodopsin-like | plasma membrane |
| | | chemokine (C-C motif) | cytosolic calcium ion | receptor activity | |
| | | receptor 2 isoform B | concentration elevation; | | |
| | | | JAK-STAT cascade; G- | | |
| | | | protein coupled receptor | | |
| | | | protein signaling pathway; | | |
| | | | chemotaxis; cellular | | |
| | | | defense response; | | |
| | | | invasive growth; | | |
| | | | inflammatory response; | | |
| | | | antimicrobial humoral | | |
| | | | response (sensu | | |
| | | | Vertebrata) | | |
| GLI pathogenesis-related 1 | NM_006851 | glioma pathogenesis- | pathogenesis | | extracellular |
| (glioma) | | related protein | | | |
| type I transmembrane C-type lectin | NM_014880 | type I transmembrane | heterophilic cell adhesion | sugar binding; receptor | integral to membrane |
| receptor DCL-1 | | C-type lectin receptor | | activity | |
| | | DCL-1 | | | |
| hypothetical protein FLJ32115 | NM_152321 | hypothetical protein | | oxidoreductase activity, | |
| | | FLJ32115 | | acting on single donors | |
| | | | | with incorporation of | |
| | | | | molecular oxygen, | |
| | | | | incorporation of two | |
| | | | | atoms of oxygen | |
| unnamed protein product; Homo | XM_370932 | | | | |
| sapiens cDNA FLJ39639 fis, clone | | | | | |
| SMINT2003340.; hypothetical | | | | | |
| protein FLJ39639 | | | | | |
| HSPC063 protein | NM_014155 | HSPC063 protein | | | |
| CTD (carboxy-terminal domain, | NM_005730 | nuclear LIM interactor- | oncogenesis | GO: 5625; soluble | soluble fraction |
| RNA polymerase II, polypeptide A) | | interacting factor 2 | | fraction; | |
| small phosphatase 2 | | | | predicted/computed | |
| heat shock 70 kDa protein 1-like | NM_005527 | heat shock 70 kDa | | ATP binding; heat shock | GO: 3773; heat shock |
| | | protein 1-like | | protein activity | protein; predicted/computed |
| karyopherin alpha 1 (importin alpha | NM_002264 | karyopherin alpha 1 | regulation of DNA | nuclear localization | nuclear pore; cytoplasm; |
| 5) | | | recombination; NLS- | sequence binding; | nucleus |
| | | | bearing substrate-nucleus | protein transporter | |
| | | | import; intracellular protein | activity; protein binding | |
| | | | transport | | |
| regulator of G-protein signalling 18 | NM_130782 | regulator of G-protein | signal transduction | signal transducer activity | |
| | | signalling 18 | | | |
| regulator of G-protein signalling 2, | NM_002923 | regulator of G-protein | regulation of G-protein | GTPase activator | 157; peripheral plasma |
| 24 kDa | | signalling 2, 24 kDa | coupled receptor protein | activity; calmodulin | membrane protein; |
| | | | signaling pathway; cell | binding; signal | predicted/computed |
| | | | cycle; signal transduction | transducer activity | |
| HIV-1 rev binding protein 2 | NM_007043 | HIV-1 rev binding | | | |
| | | protein 2 | | | |
| HIV-1 rev binding protein 2 | NM_007043 | HIV-1 rev binding | | | |
| | | protein 2 | | | |
| Homo sapiens mRNA; cDNA | AL137346 | | | | |
| DKFZp761M0111 (from clone | | | | | |
| DKFZp761M0111) | | | | | |
| HIV-1 rev binding protein 2 | NM_007043 | HIV-1 rev binding | | | |
| | | protein 2 | | | |
| GLI pathogenesis-related 1 | NM_006851 | glioma pathogenesis- | pathogenesis | | extracellular |
| (glioma) | | related protein | | | |
| adaptor-related protein complex 1, | NM_003916 | adaptor-related protein | endocytosis; intracellular | protein transporter | Golgi trans face; clathrin |
| sigma 2 subunit | | complex 1 sigma 2 | protein transport | activity | adaptor; coated pit; AP-1 |
| | | subunit | | | adaptor complex; clathrin |
| | | | | | vesicle coat |
| membrane-spanning 4-domains, | NM_021201 | membrane-spanning 4- | | receptor activity | integral to membrane |
| subfamily A, member 7 | | domains, subfamily A, | | | |
| | | member 7 | | | |
| DKFZP586A0522 protein | NM_014033 | DKFZP586A0522 | | | |
| | | protein | | | |
| Homo sapiens cDNA FLJ39934 fis, | AL831930 | hypothetical protein | | | |
| clone SPLEN2021458, weakly | | | | | |
| similar to Mus musculus mdgl-1 | | | | | |
| mRNA. | | | | | |
| Homo sapiens transcribed | AI732570 | | | | |
| sequences | | | | | |
| Homo sapiens pp12719 mRNA, | AF318328 | | | | |
| complete cds | | | | | |
| ATP-binding cassette, sub-family C | NM_005688 | ATP-binding cassette, | transport; small molecule | nucleotide binding | integral to plasma |
| (CFTR/MRP), member 5 | | sub-family C, member 5 | transport | activity; organic anion | membrane; membrane |
| | | | | transporter activity; ATP | fraction |
| | | | | binding activity; ATP- | |
| | | | | binding cassette (ABC) | |
| | | | | transporter activity; | |
| | | | | multidrug transporter | |
| | | | | activity | |
| retinoid binding protein 7 | NM_052960 | retinoid binding protein 7 | transport | lipid binding activity; | |
| | | | | transporter activity; | |
| | | | | retinol binding activity | |
| oxysterol binding protein-like 8 | NM_020841 | oxysterol-binding | | | |
| | | protein-like protein 8 | | | |
| hypothetical protein FLJ37953 | NM_152382 | hypothetical protein | | | |
| | | FLJ37953 | | | |
| RNA-binding region (RNP1, RRM) | NM_153020 | hypothetical protein | | | |
| containing 6 | | FLJ30829 | | | |
| Homo sapiens, clone | BC043219 | | | | |
| IMAGE: 5295326, mRNA | | | | | |
| Homo sapiens mRNA; cDNA | BX648714 | | | | |
| DKFZp686D21117 (from clone | | | | | |
| DKFZp686D21117) | | | | | |
| Homo sapiens mRNA for | AB028949 | KIAA1026 protein | GO: 6470 protein | | GO: 8181 tumor suppressor |
| KIAA1026 protein, partial cds. | | | dephosphorylation | | (not recorded) GO: 163 |
| | | | (predicted/computed) | | protein phosphatase type 1 |
| | | | | | (predicted/computed) |
| | | | | | GO: 8598 protein |
| | | | | | phosphatase type 1 catalyst |
| | | | | | (not recorded) |
| protein kinase, AMP-activated, | NM_017431 | protein kinase, AMP- | protein kinase cascade; | SNF1A/AMP-activated | GO: 4679; SNF1A/AMP- |
| gamma 3 non-catalytic subunit | | activated, gamma 3 | energy pathways; fatty | protein kinase activity | activated protein kinase |
| | | non-catalytic subunit | acid biosynthesis | | activity traceable author |
| | | | | | statement |
| pleckstrin homology domain | NM_017934 | pleckstrin homology | | | |
| interacting protein | | domain interacting | | | |
| | | protein | | | |
| hypothetical protein | NM_017566 | hypothetical protein | | | |
| DKFZp434G0522 | | DKFZp434G0522 | | | |
| Homo sapiens clone FLB2543 | AF113675 | CCR4-NOT transcription | | | |
| | | complex, subunit 2 | | | |
| deoxythymidylate kinase | NM_012145 | deoxythymidylate kinase | cell cycle; DNA | thymidylate kinase | GO: 16301; kinase activity; |
| (thymidylate kinase) | | (thymidylate kinase) | metabolism; dTDP | activity; ATP binding; | inferred from electronic |
| | | | biosynthesis; dTTP | transferase activity | annotation GO: 16740 |
| | | | biosynthesis; nucleotide | | transferase activity; inferred |
| | | | biosynthesis | | from electronic annotation |
| | | | | | GO: 4798; thymidylate kinase |
| | | | | | activity; traceable author |
| | | | | | statement GO: 5524; ATP |
| | | | | | binding; inferred from |
| | | | | | electronic annotation |
| transient receptor potential cation | NM_017662 | transient receptor | | 5216; ion channel; | |
| channel, subfamily M, member 6 | | potential cation channel, | | extended:inferred from | |
| | | subfamily M, member 6 | | sequence similarity; | |
| | | | | ion_trans; 0.018 | |
| Rho guanine nucleotide exchange | NM_145735 | Rho guanine nucleotide | signal transduction | guanyl-nucleotide | |
| factor (GEF) 7 | | exchange factor 7 | | exchange factor activity | |
| | | isoform a; Rho guanine | | | |
| | | nucleotide exchange | | | |
| | | factor 7 isoform b | | | |
| keratin 4 | NM_002272 | keratin 4 | cytoskeleton organization | structural molecule | intermediate filament |
| | | | and biogenesis | activity | |
| Homo sapiens mRNA; cDNA | AL833240 | | | | |
| DKFZp761P2319 (from clone | | | | | |
| DKFZp761P2319) | | | | | |
| Homo sapiens transcribed | BM676479 | | | | |
| sequences | | | | | |
| proprotein convertase | NM_006200 | proprotein convertase | | | |
| subtilisin/kexin type 5 | | subtilisin/kexin type 5 | | | |
| | | preproprotein | | | |
| reticulon 1 | NM_021136 | reticulon 1 | signal transduction | molecular_function | endoplasmic reticulum; |
| | | | neuron differentiation | unknown; signal | integral to endoplasmic |
| | | | | transducer activity | reticulum membrane |
| tubulin, beta 1 | NM_030773 | beta tubulin 1, class VI | microtubule-based | GTP binding; structural | microtubule |
| | | | movement | molecule activity | |
| Homo sapiens cDNA FLJ32207 fis, | AK056769 | | | | |
| clone PLACE6003204. | | | | | |
| similar to junction-mediating and | AK126887 | KIAA1971 protein | electron transport | electron transporter | |
| regulatory protein p300 JMY | | | | activity | |
| Homo sapiens cDNA FLJ37963 fis, | AK095282 | | | | |
| clone CTONG2009689. | | | | | |
| likely ortholog of mouse IRA1 | NM_024665 | nuclear receptor co- | | | |
| protein | | repressor/HDAC3 | | | |
| | | complex subunit | | | |
| chromosome 9 open reading frame | NM_030814 | chromosome 9 open | | | |
| 45 | | reading frame 45 | | | |
| natural killer cell group 7 sequence | NM_005601 | natural killer cell group 7 | | GO: 5887; integral | integral to plasma |
| | | sequence | | plasma membrane | membrane |
| | | | | protein; | |
| | | | | predicted/computed | |
| granzyme B (granzyme 2, cytotoxic | NM_004131 | granzyme B precursor | proteolysis and | trypsin activity; | cytoplasm |
| T-lymphocyte-associated serine | | | peptidolysis; apoptosis; | granzyme B activity; | |
| esterase 1) | | | cytolysis | chymotrypsin activity; | |
| | | | | hydrolase activity | |
| SH2 domain protein 2A | NM_003975 | SH2 domain protein 2A | intracellular signaling | 5070; SH3/SH2 adaptor | 5737; cytoplasm; |
| | | | cascade; angiogenesis | protein; | experimental evidence; |
| | | | | predicted/computed | 5625; soluble fraction; |
| | | | | | experimental evidence |
| dual specificity phosphatase 2 | NM_004418 | dual specificity | inactivation of MAPK; | protein | nucleus |
| | | phosphatase 2 | protein amino acid | tyrosine/threonine | |
| | | | dephosphorylation | phosphatase activity; | |
| | | | | protein tyrosine | |
| | | | | phosphatase activity | |
| chemokine (C-C motif) ligand 4 | NM_002984 | chemokine (C-C motif) | response to virus; | receptor signaling | extracellular space |
| | | ligand 4 precursor | establishment and/or | protein tyrosine kinase | |
| | | | maintenance of cell | activity; chemokine | |
| | | | polarity; cell growth and/or | activity | |
| | | | maintenance; chemotaxis; | | |
| | | | cell adhesion; immune | | |
| | | | response; cell motility; | | |
| | | | signal transduction; cell- | | |
| | | | cell signaling; | | |
| | | | inflammatory response; | | |
| | | | viral genome replication | | |
| Homo sapiens cDNA FLJ38531 fis, | AK095850 | Unknown (protein for | | | |
| clone HCHON2001050. | | IMAGE: 2822295) | | | |
| Homo sapiens partial mRNA; ID | R01220 | | | | |
| YG31-1, YG81-3B, LG43-4B2 | | | | | |
| hypothetical protein MGC29671 | NM_182538 | hypothetical protein | | | |
| | | MGC29671 | | | |
| Homo sapiens, clone | BC043400 | | | | |
| IMAGE: 6016214, mRNA | | | | | |
| hypothetical protein LOC90637 | NM_182491 | hypothetical protein | electron transport | electron transporter | |
| | | LOC90637 | | activity; | |
| | | | | molecular_function | |
| | | | | unknown | |
| cell division cycle associated 7 | NM_031942 | cell division cycle | | | |
| | | associated protein 7 | | | |
| | | isoform 1; cell division | | | |
| | | cycle associated protein | | | |
| | | 7 isoform 2 | | | |
| hypothetical protein MGC24665 | NM_152308 | hypothetical protein | | | |
| | | MGC24665 | | | |
| interferon, gamma | NM_000619 | interferon, gamma | cell surface receptor | interferon-gamma | extracellular |
| | | | linked signal transduction; | receptor binding; | |
| | | | immune response; cell | cytokine activity | |
| | | | motility; cell-cell signaling; | | |
| | | | regulation of cell growth | | |
| regulator of G-protein signalling 1 | NM_002922 | regulator of G-protein | G-protein signaling, | GTPase activator | plasma membrane |
| | | signalling 1 | adenylate cyclase | activity; calmodulin | |
| | | | inhibiting pathway; | binding; signal | |
| | | | immune response; signal | transducer activity | |
| | | | transduction; B-cell | | |
| | | | activation | | |
| hypothetical protein FLJ12150 | NM_024736 | hypothetical protein | | | |
| | | FLJ12150 | | | |
| methylene tetrahydrofolate | NM_006636 | methylene | one-carbon compound | methenyltetrahydrofolate | mitochondrion |
| dehydrogenase (NAD+ | | tetrahydrofolate | metabolism; folic acid and | cyclohydrolase activity; | |
| dependent), | | dehydrogenase 2 | derivative biosynthesis | electron transporter | |
| methenyltetrahydrofolate | | precursor | | activity; magnesium ion | |
| cyclohydrolase | | | | binding; | |
| | | | | methylenetetrahydrofolate | |
| | | | | dehydrogenase (NAD) | |
| | | | | activity; oxidoreductase | |
| | | | | activity | |
| F-box only protein 6 | NM_018438 | F-box only protein 6 | proteolysis and | ubiquitin conjugating | GO: 4842; ubiquitin - protein |
| | | | peptidolysis | enzyme activity; | ligase; not recorded |
| | | | | ubiquitin-protein ligase | GO: 4840; ubiquitin |
| | | | | activity | conjugating enzyme; |
| | | | | | predicted/computed |
| bone marrow stromal cell antigen 2 | NM_004335 | bone marrow stromal | humoral immune | GO: 5887; integral | integral to plasma |
| | | cell antigen 2 | response; development; | plasma membrane | membrane |
| | | | cell proliferation; cell-cell | protein; | |
| | | | signaling | predicted/computed | |
| hypothetical protein FLJ12770 | NM_032174 | hypothetical protein | anion transport | voltage-dependent ion- | mitochondrial outer |
| | | FLJ12770 | | selective channel activity | membrane |
| neuritin 1 | NM_016588 | neuritin precursor | | | |
| metallothionein 1H | NM_005951 | metallothionein 1H | | metal ion binding | GO: 5505; heavy metal |
| | | | | | binding; not recorded |
| metallothionein 1G | NM_005950 | metallothionein 1G | | metal ion binding | GO: 5505; heavy metal |
| | | | | | binding; not recorded |
| metallothionein 1H | NM_005951 | metallothionein 1H | | metal ion binding | |
| metallothionein 2A | NM_175617 | metallothionein 1E | heavy metal ion transport | heavy metal ion | |
| | | | | transporter activity | |
| | AL031602 | | | | |
| metallothionein 1X | NM_005952 | metallothionein 1X | response to metal ion | metal ion binding | cytoplasm |
| metallothionein 1X | NM_005952 | metallothionein 1X | response to metal ion | metal ion binding | GO: 5505; heavy metal |
| | | | | | binding; not recorded |
| metallothionein 1F (functional) | NM_005949 | metallothionein 1F | biological_process | copper ion binding; zinc | cytoplasm |
| | | | unknown | ion binding; metal ion | |
| | | | | binding; cadmium ion | |
| | | | | binding | |
| brain acyl-CoA hydrolase | NM_181862 | brain acyl-CoA | lipid metabolism | serine esterase activity; | cytoplasm |
| | | hydrolase isoform | | acyl-CoA binding; | |
| | | hBACHa; brain acyl-CoA | | hydrolase activity; | |
| | | hydrolase isoform | | palmitoyl-CoA hydrolase | |
| | | hBACHa/X; brain acyl- | | activity | |
| | | CoA hydrolase isoform | | | |
| | | hBACHa/Xi; brain acyl- | | | |
| | | CoA hydrolase isoform | | | |
| | | hBACHb; brain acyl-CoA | | | |
| | | hydrolase isoform | | | |
| | | hBACHc; brain acyl-CoA | | | |
| | | hydrolase isoform | | | |
| | | hBACHd | | | |
| argininosuccinate synthetase | NM_054012 | argininosuccinate | urea cycle; arginine | ATP binding activity; | cytoplasm |
| | | synthetase | biosynthesis | argininosuccinate | |
| | | | | synthase activity; ligase | |
| | | | | activity | |
| RAD51 homolog (RecA homolog, | NM_002875 | RAD51 homolog protein | mitotic recombination; | DNA dependent ATPase | nucleus |
| E. coli) (S. cerevisiae) | | isoform 1; RAD51 | meiotic recombination; | activity; damaged DNA | |
| | | homolog protein isoform 2 | DNA repair | binding; nucleotide | |
| | | | | binding; ATP binding | |
| v-jun sarcoma virus 17 oncogene | NM_002228 | v-jun avian sarcoma | cell growth and/or | RNA polymerase II | nuclear chromosome |
| homolog (avian) | | virus 17 oncogene | maintenance; regulation of | transcription factor | |
| | | homolog | transcription, DNA- | activity | |
| | | | dependent | | |
| chromosome 14 open reading | NM_031427 | chromosome 14 open | | | |
| frame 168 | | reading frame 168 | | | |
| ets variant gene 5 (ets-related | NM_004454 | ets variant gene 5 (ets- | regulation of transcription, | transcription factor | nucleus |
| molecule) | | related molecule) | DNA-dependent | activity | |
| metallothionein 1K | NM_176870 | metallothionein 1K | | | |
| Jun dimerization protein p21SNFT | NM_018664 | Jun dimerization protein | response to | transcription co- | nucleus |
| | | p21SNFT | pest/pathogen/parasite; | repressor activity; | |
| | | | regulation of transcription, | transcription factor | |
| | | | DNA-dependent; | activity | |
| | | | transcription from Pol II | | |
| | | | promoter | | |
| potassium channel tetramerisation | NM_023930 | hypothetical protein | potassium ion transport | voltage-gated potassium | membrane; voltage-gated |
| domain containing 14 | | MGC2376 | | channel activity; protein | potassium channel complex |
| | | | | binding | |
| chemokine (C-C motif) ligand 2 | NM_002982 | small inducible cytokine | response to pathogenic | chemokine activity; | membrane; extracellular |
| | | A2 precursor | bacteria; JAK-STAT | protein kinase activity | space |
| | | | cascade; G-protein | | |
| | | | signaling, coupled to cyclic | | |
| | | | nucleotide second | | |
| | | | messenger; chemotaxis; | | |
| | | | protein amino acid | | |
| | | | phosphorylation; calcium | | |
| | | | ion homeostasis; humoral | | |
| | | | immune response; cell | | |
| | | | adhesion; cell-cell | | |
| | | | signaling; inflammatory | | |
| | | | response; organogenesis; | | |
| | | | viral genome replication | | |
| IQ motif containing GTPase | NM_178229 | IQ motif containing | small GTPase mediated | Ras GTPase activator | |
| activating protein 3 | | GTPase activating | signal transduction | activity | |
| | | protein 3 | | | |
| tight junction protein 1 (zona | NM_003257 | tight junction protein 1 | intercellular junction | protein binding | septate junction; tight |
| occludens 1) | | isoform a; tight junction | assembly | | junction; membrane fraction; |
| | | protein 1 isoform b | | | plasma membrane |
| proteoglycan 2, bone marrow | NM_002728 | proteoglycan 2 | xenobiotic metabolism; | sugar binding; heparin | extracellular; cytoplasm |
| (natural killer cell activator, | | | immune response; | binding; toxin activity | |
| eosinophil granule major basic | | | inflammatory response; | | |
| protein) | | | heterophilic cell adhesion | | |
| early growth response 1 | NM_001964 | early growth response 1 | regulation of transcription, | transcription factor | nucleus |
| | | | DNA-dependent | activity | |
| Human cathepsin-L-like (CTSLL3) | L25629 | | | | |
| mRNA. | | | | | |
| chemokine (C-C motif) ligand 3 | NM_002983 | chemokine (C-C motif) | G-protein coupled receptor | chemokine activity; | soluble fraction; extracellular |
| | | ligand 3 | protein signaling pathway; | antiviral response | |
| | | | cytoskeleton organization | protein activity; signal | |
| | | | and biogenesis; | transducer activity | |
| | | | chemotaxis; calcium ion | | |
| | | | homeostasis; exocytosis; | | |
| | | | immune response; cell | | |
| | | | motility; signal | | |
| | | | transduction; cell-cell | | |
| | | | signaling; inflammatory | | |
| | | | response; antimicrobial | | |
| | | | humoral response (sensu | | |
| | | | Vertebrata); regulation of | | |
| | | | viral genome replication | | |
| cAMP responsive element | NM_183013 | cAMP responsive | signal transduction | 5515; protein binding; | nucleus |
| modulator | | element modulator | | extended:inferred from | |
| | | isoforms a-b, d-m | | electronic annotation; | |
| | | | | pKID; 4.6e−24 | |
| J domain containing protein 1 | NM_021800 | J domain containing | protein folding | chaperone activity | |
| | | protein 1 | | | |
| apolipoprotein C-I | NM_001645 | apolipoprotein C-I | lipid transport; lipid | lipid transporter activity | extracellular |
| | | precursor | metabolism; lipoprotein | | |
| | | | metabolism | | |
| olfactory receptor, family 2, | NM_012368 | olfactory receptor, family | olfaction; G-protein | olfactory receptor activity | integral to membrane |
| subfamily C, member 1 | | 2, subfamily C, member 1 | coupled receptor protein | | |
| | | | signaling pathway | | |
| apolipoprotein C-I | NM_001645 | apolipoprotein C-I | lipid transport; lipid | lipid transporter activity | extracellular |
| | | precursor | metabolism; lipoprotein | | |
| | | | metabolism | | |
| gb: BC020700.1 | BC020700 | | GO: 5978; glycogen | GO: 5792; microsome; | GO: 16787; hydrolase |
| /DB_XREF = gi: 18088393 | | | biosynthesis; inferred from | not recorded GO: 5783; | activity; inferred from |
| /TID = Hs2Affx.1.389 /CNT = 1 | | | electronic annotation | endoplasmic reticulum; | electronic annotation |
| /FEA = FLmRNA /TIER = FL /STK = 1 | | | | inferred from electronic | GO: 4346; glucose-6- |
| /NOTE = sequence(s) not in | | | | annotation GO: 16021; | phosphatase activity; |
| UniGene /DEF = Homo sapiens, | | | | integral to membrane; | traceable author statement |
| clone MGC: 22459 | | | | inferred from electronic | |
| IMAGE: 4722671, mRNA, complete | | | | annotation | |
| cds. /PROD = Unknown (protein for | | | | | |
| MGC: 22459) /FL = gb: BC020700.1 | | | | | |
| Homo sapiens, clone | BC039329 | | | | |
| IMAGE: 5267606, mRNA | | | | | |
| v-jun sarcoma virus 17 oncogene | NM_002228 | v-jun avian sarcoma | cell growth and/or | RNA polymerase II | nuclear chromosome |
| homolog (avian) | | virus 17 oncogene | maintenance; regulation of | transcription factor | |
| | | homolog | transcription, DNA- | activity | |
| | | | dependent | | |
| v-maf musculoaponeurotic | NM_012323 | transcription factor | regulation of transcription, | DNA binding; | nucleus |
| fibrosarcoma oncogene homolog F | | MAFF | DNA-dependent | transcription co-activator | |
| (avian) | | | | activity | |
| chemokine (C-C motif) receptor- | NM_003965 | chemokine (C-C motif) | G-protein coupled receptor | chemokine receptor | integral to plasma |
| like 2 | | receptor-like 2 | protein signaling pathway; | activity | membrane |
| | | | chemotaxis; antimicrobial | | |
| | | | humoral response (sensu | | |
| | | | Invertebrata) | | |
| H factor (complement)-like 1 | NM_002113 | H factor (complement)- | | | |
| | | like 1 | | | |
| suppressor of cytokine signaling 1 | NM_003745 | suppressor of cytokine | JAK-STAT cascade; | protein kinase inhibitor | cytoplasm |
| | | signaling 1 | intracellular signaling | activity | |
| | | | cascade; regulation of cell | | |
| | | | growth | | |
| H factor 1 (complement) | NM_000186 | H factor 1 (complement) | complement activation, | complement activity | extracellular space |
| | | | alternative pathway | | |
| zinc finger protein, subfamily 1A, 4 | NM_022465 | zinc finger protein, | | | |
| (Eos) | | subfamily 1A, 4 | | | |
| synaptopodin 2 | AL833547 | | | | |
| Siah-interacting protein | NM_014412 | calcyclin binding protein | | | |
| KIAA0478 gene product | NM_014870 | KIAA0478 gene product | regulation of transcription, | protein binding; DNA | nucleus |
| | | | DNA-dependent | binding | |
| microtubule-associated protein 1B | NM_005909 | microtubule-associated | microtubule-based | structural molecule | microtubule associated |
| | | protein 1B isoform 1; | process | activity | complex |
| | | microtubule-associated | | | |
| | | protein 1B isoform 2 | | | |
| ectonucleoside triphosphate | NM_001248 | ectonucleoside | | apyrase activity; | integral to membrane |
| diphosphohydrolase 3 | | triphosphate | | magnesium ion binding; | |
| | | diphosphohydrolase 3 | | hydrolase activity | |
| ym42f03.s1 Soares infant brain | H17132 | | | | |
| 1NIB Homo sapiens cDNA clone | | | | | |
| IMAGE: 50973 3′, mRNA | | | | | |
| sequence. | | | | | |
| hypothetical protein LOC339807 | XM_379099 | | | | |
| hypothetical protein BC008988 | NM_138379 | hypothetical protein | | | |
| | | BC008988 | | | |
| Homo sapiens cDNA FLJ14061 fis, | AK024123 | | | | |
| clone HEMBB1000749. | | | | | |
| FERM, RhoGEF (ARHGEF) and | NM_005766 | FERM, RhoGEF, and | | Rho guanyl-nucleotide | cytoskeleton |
| pleckstrin domain protein 1 | | pleckstrin domain | | exchange factor activity | |
| (chondrocyte-derived) | | protein 1 | | | |
| ankyrin repeat domain 1 (cardiac | NM_014391 | cardiac ankyrin repeat | defense response; signal | DNA binding activity | nucleus |
| muscle) | | protein | transduction | | |
| Homo sapiens cDNA FLJ35233 fis, | AK092552 | | | | |
| clone PROST2001540. | | | | | |
| RNA terminal phosphate cyclase- | NM_005772 | RNA cyclase homolog | biological_process | RNA-3′-phosphate | nucleolus |
| like 1 | | | unknown | cyclase activity | |
| 2′-5′-oligoadenylate synthetase 3, | NM_006187 | 2′-5′oligoadenylate | nucleobase, nucleoside, | ATP binding; antiviral | microsome |
| 100 kDa | | synthetase 3 | nucleotide and nucleic | response protein | |
| | | | acid metabolism; immune | activity; RNA binding; | |
| | | | response | transferase activity; | |
| | | | | nucleotidyltransferase | |
| | | | | activity | |
| cyclin-E binding protein 1 | NM_016323 | cyclin-E binding protein 1 | ubiquitin cycle; regulation | ubiquitin-protein ligase | intracellular |
| | | | of CDK activity | activity | |
| chromosome 1 open reading frame | NM_006820 | histocompatibility 28 | | | |
| 29 | | | | | |
| interferon, alpha-inducible protein | NM_005101 | interferon, alpha- | immune response; cell-cell | protein binding | extracellular space; |
| (clone IFI-15K) | | inducible protein (clone | signaling | | cytoplasm |
| | | IFI-15K) | | | |
| XIAP associated factor-1 | NM_017523 | XIAP associated factor- | | zinc ion binding | |
| | | 1 isoform 1; XIAP | | | |
| | | associated factor-1 | | | |
| | | isoform 2 | | | |
| hypothetical protein FLJ22693 | NM_022750 | zinc finger CCCH type | | nucleic acid binding | |
| | | domain containing 1 | | | |
| 2′-5′-oligoadenylate synthetase 2, | NM_002535 | 2′-5′oligoadenylate | nucleobase, nucleoside, | ATP binding activity; | membrane; microsome |
| 69/71 kDa | | synthetase 2 isoform | nucleotide and nucleic | antiviral response | |
| | | p69; 2′-5′oligoadenylate | acid metabolism; immune | protein activity; RNA | |
| | | synthetase 2 isoform | response | binding activity; | |
| | | p71 | | transferase activity; | |
| | | | | nucleotidyltransferase | |
| | | | | activity | |
| lymphocyte antigen 6 complex, | NM_002346 | lymphocyte antigen 6 | defense response; cell | GO: 5887; integral | membrane; integral to |
| locus E | | complex, locus E | surface receptor linked | plasma membrane | plasma membrane |
| | | | signal transduction | protein; | |
| | | | | predicted/computed | |
| 2′-5′-oligoadenylate synthetase 2, | NM_002535 | 2′-5′oligoadenylate | nucleobase, nucleoside, | ATP binding activity; | membrane; microsome |
| 69/71 kDa | | synthetase 2 isoform | nucleotide and nucleic | antiviral response | |
| | | p69; 2′-5′oligoadenylate | acid metabolism; immune | protein activity; RNA | |
| | | synthetase 2 isoform | response | binding activity; | |
| | | p71 | | transferase activity; | |
| | | | | nucleotidyltransferase | |
| | | | | activity | |
| DNA polymerase-transactivated | NM_015535 | DNA polymerase- | | | |
| protein 6 | | transactivated protein 6 | | | |
| ubiquitin specific protease 18 | NM_017414 | ubiquitin specific | ubiquitin-dependent | ubiquitin-specific | nucleus |
| | | protease 18 | protein catabolism | protease activity; | |
| | | | | cysteine-type | |
| | | | | endopeptidase activity; | |
| | | | | ubiquitin thiolesterase | |
| | | | | activity; hydrolase | |
| | | | | activity | |
| Mov10, Moloney leukemia virus 10, | NM_020963 | Mov10, Moloney | | | |
| homolog (mouse) | | leukemia virus 10, | | | |
| | | homolog | | | |
| synonyms: LAMP, DCLAMP, | NM_014398 | lysosomal-associated | cell proliferation | GO: 5765; lysosomal | lysosomal membrane |
| TSC403, DC-LAMP; Homo | | membrane protein 3 | | membrane; | |
| sapiens lysosomal-associated | | | | predicted/computed | |
| membrane protein 3 (LAMP3), | | | | | |
| mRNA. | | | | | |
| viperin | NM_080657 | viperin | | | |
| Homo sapiens transcribed | BG205162 | | | | |
| sequences | | | | | |
| hypothetical protein BC009980 | NM_138433 | hypothetical protein | | | |
| | | BC009980 | | | |
| transmembrane 6 superfamily | NM_023003 | transmembrane 6 | | | |
| member 1 | | superfamily member 1 | | | |
| hemoglobin, zeta | NM_005332 | zeta globin | oxygen transport | oxygen transporter | hemoglobin complex |
| | | | | activity | |
| carbohydrate sulfotransferase 10 | NM_004854 | HNK-1 sulfotransferase | cell adhesion | sulfotransferase activity | Golgi apparatus; membrane |
| | | | | | fraction |
| zinc finger, CW-type with PWWP | NM_017984 | zinc finger, CW-type | | | |
| domain 1 | | with PWWP domain 1 | | | |
| alpha-2-macroglobulin | NM_000014 | alpha 2 macroglobulin | intracellular protein | protein carrier activity; | GO: 4866; proteinase |
| | | precursor | transport | serine protease inhibitor | inhibitor; not recorded |
| | | | | activity; wide-spectrum | GO: 8320; protein carrier; not |
| | | | | protease inhibitor activity | recorded |
| phospholipase C, delta 3 | NM_133373 | phospholipase C delta 3 | lipid metabolism; | calcium ion binding; | GO: 4629; PI-PLC-X; |
| | | | intracellular signaling | phosphoinositide | phospholipase C activity; |
| | | | cascade | phospholipase C activity | 1.9e−76; extended:inferred |
| | | | | | from sequence similarity |
| Homo sapiens cDNA: FLJ22620 | AK026273 | | | | |
| fis, clone HSI05629 | | | | | |
| Homo sapiens transcribed | BM543270 | | | | |
| sequence with weak similarity to | | | | | |
| protein ref: NP_055301.1 | | | | | |
| (H. sapiens) neuronal thread | | | | | |
| protein [Homo sapiens] | | | | | |
| Homo sapiens, clone | BE791720 | | | | |
| IMAGE: 6454649, mRNA | | | | | |
| myosin light chain kinase (MLCK) | NM_182493 | myosin light chain | protein amino acid | ATP binding; protein | GO: 4672; pkinase; protein |
| | | kinase (MLCK) | phosphorylation | serine/threonine kinase | kinase activity; 6.3e−88; |
| | | | | activity; transferase | extended:inferred from |
| | | | | activity | electronic annotation |
| Homo sapiens, clone | BI827840 | | | | |
| IMAGE: 5166083, mRNA | | | | | |

Table 2 below, lists the accession numbers, nucleic acid sequences, and protein sequences of several of the upregulated metallothionein family members.

TABLE 2
Selected Metallothionein genes upregulated in high risk septic shock

| | | | | PROTEIN |
|---|---|---|---|---|
| | | GENE SEQ | CDS SEQ | SEQ ID |
| Name | CDS ACC# | ID NO: | ID NO: | NO: |
| metallothionein 1E | NM_175617 | 1 | 2 | 3 |
| metallothionein 1F | NM_005949 | 4 | 5 | 6 |
| metallothionein 1H | NM_005951 | 7 | 8 | 9 |
| metallothionein 1G | NM_005950 | 10 | 11 | 12 |
| metallothionein 1X | NM_005952 | 13 | 14 | 15 |
| metallothionein 1K | NM_176870 | 16 | 17 | 18 |

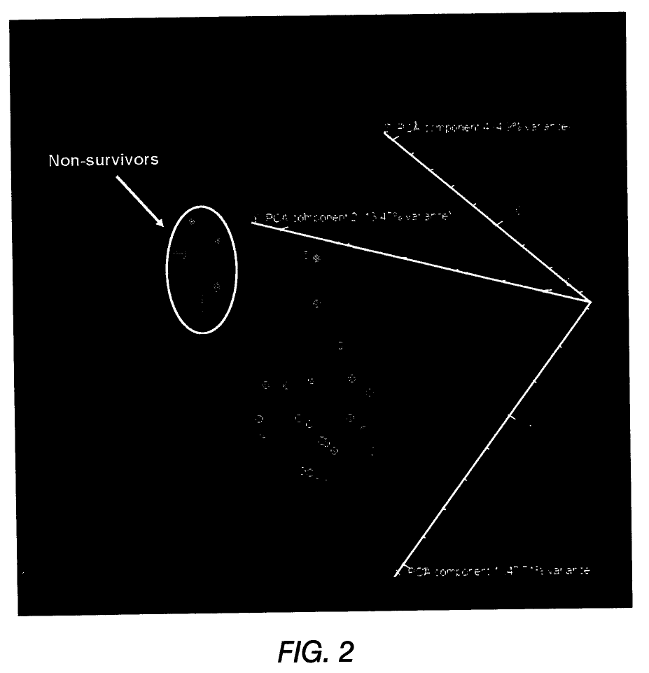

Principle component analysis was used to compare the expression of the 400 differentially expressed genes, as shown in FIG. 2. This analysis was based on the relative strength of different expression patterns that are activated or repressed in a given patient. These relative strengths were quantified for each patient and are graphed according to the strength of three principal components for each patient in the 3-dimensional graph. The pattern of expression of the 400 predictor genes in the septic shock patients that succumbed is different than in those who survived. The data for the patients that succumbed (shown in red) clusters in a region of the graph that reflects the altered expression pattern of many genes.

The 400 genes that were found in the analysis serve as very strong markers for predicting high risk patients, although there are also other genes that were found to be capable of predicting a high risk outcome.

The separation of the patients that would later succumb is based on the induction of the metallothionein genes and on the failure to activate the expression of the genes that are much more strongly induced in the surviving septic shock patients. Thus, the genes that are strongly induced in patients who were able to recover are part of the body's protective response.

In addition to being a predictor of death, the MT genes were also an early predictor of death. Samples that were obtained on the first day of septic shock were already positive for metallothionein gene expression. Children with septic shock who progressed to death had high expression levels of the MT gene family members, whereas control patients and patients that survived septic shock did not. These data show that MT, in particular, is a biomarker for early prediction of death in pediatric septic shock.

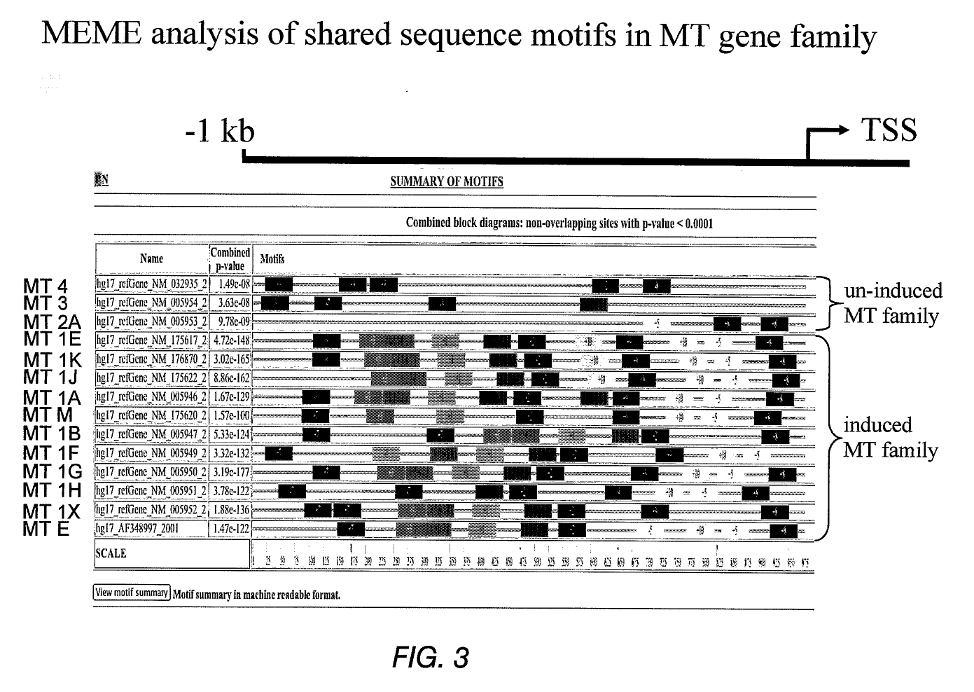

Metallothionein family proteins are ubiquitous in eukaryotes. Four metallothionein genes, MT-1, MT-2, MT-3, and MT-4, have been extensively characterized. MT-1 and MT-2 have been found to be induced by a variety of metals, drugs, and inflammatory mediators. The MT family members are low molecular weight, cysteine-rich proteins that are localized in the cytosol. These proteins are capable of binding to metals, and also exhibit redox capabilities. One role of the MT proteins is the protection from metal toxicity, possibly by binding and sequestration of excess metal ions. Other roles for metallothionein are also indicated. FIG. 3 is a diagram showing a summary of motifs in the promoter region of the genes encoding various MT family members.

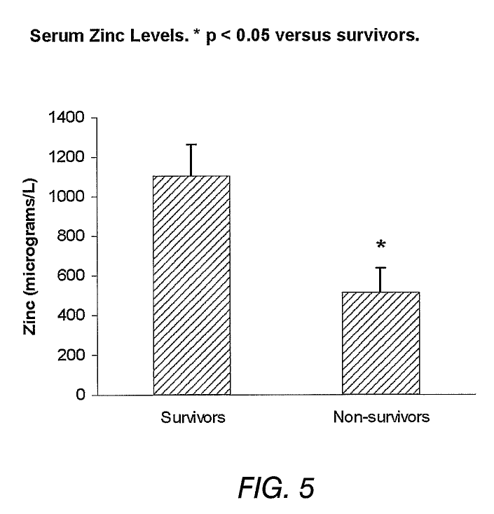

The consequences of metallothionein gene and protein induction can be anticipated to lead to changes in zinc levels (as shown in FIG. 5), the levels of other proteins, and changes in the activation of many other genes and alterations in the cell and outside of the cell. Any of these serve to indicate that the patient is in extreme risk and needs urgent treatment.

In addition to the metallothionein family, many other genes were found to be upregulated in the high risk group of septic shock individuals. A partial list of these upregulated genes is listed below in Table 3. Thus, in some embodiments of the invention, a set of signature genes that is upregulated in individuals at high risk of death is provided. Some of these signature genes can be useful as early predictors of the high risk of death from septic shock.

TABLE 3
Additional selected genes highly activated in non-survivors

| | | GENE | CDS | PROTEIN |
|---|---|---|---|---|
| NAME | ACC # | SEQ ID | SEQ ID | SEQ ID |
| granzyme B (granzyme 2, | NM_004131 | 19 | 20 | 21 |
| cytotoxic T-lymphocyte- | | | | |
| associated | | | | |
| serine esterase 1) | | | | |
| dual specificity phosphatase | NM_004418 | 22 | 23 | 24 |
| 2 | | | | |
| regulator of G-protein | NM_002922 | 25 | 26 | 27 |
| signalling 1 | | | | |
| V-Jun | NM_002228 | 28 | 29 | 30 |
| Jun dimerization protein | NM_018664 | 31 | 32 | 33 |
| chemokine ligand 2 | NM_002982 | 34 | 35 | 36 |
| chemokine ligand 3 | NM_002983 | 37 | 38 | 39 |
| chemokine (C-C motif) | NM_003965 | 40 | 41 | 42 |
| receptor-like 2 | | | | |
| cAMP responsive element | NM_183013 | 43 | 44 | 45 |
| modulator | | | | |
| complement factor H | NM_000186 | 46 | 47 | 48 |
| SOCS 1 | NM_003745 | 49 | 50 | 51 |
| Interferon-gamma | NM_000619 | 52 | 53 | 54 |
| interferon regulatory factor | NM_004031 | 55 | 56 | 57 |
| 7 | | | | |

Several genes were also found to be repressed or not activated in the non-survivors in comparison to the survivors. Table 4, below, lists a summary of these genes. A knowledge of genes that are downregulated in the non-survivors can also be useful for diagnosis of the severity of a case of septic shock.

TABLE 4
Selected genes repressed or not activated in non-survivors

| | | GENE | CDS | PROTEIN |
|---|---|---|---|---|
| NAME | ACC # | SEQ ID | SEQ ID | SEQ ID |
| Retinoid X receptor | NM_021976 | 58 | 59 | 60 |
| Caspase recruitment domain family, | NM_021209 | 61 | 62 | 63 |
| (member 12) | | | | |
| Caspase 2 | NM_032982 | 64 | 65 | 66 |
| AtP binding cassette | NM_000352 | 67 | 68 | 69 |
| Factor V Leiden | NM_000130 | 70 | 71 | 72 |
| Protein phosphatase 1 (3D) | NM_006242 | 73 | 74 | 75 |
| Protein kinase C | NM_002738 | 76 | 77 | 78 |
| Zinc finger protein 36 | BX640646 | 79 | 80 | 81 |
| Zinc finger protein 238 | NM_006352 | 82 | 83 | 84 |
| Solute carrier family 30 (zinc | NM_017964 | 85 | 86 | 87 |
| transporter) | | | | |
| Zinc finger protein ZNF-U69274 | NM_014415 | 88 | 89 | 90 |
| Hypothetical protein FLJ39485 (zinc | NM_175920 | 91 | 92 | 93 |
| ion binding) | | | | |
| Ret finger protein 2 (zinc ion binding) | NM_052811 | 94 | 95 | 96 |
| RAB guanine nucleotide exchange | NM_014504 | 97 | 98 | 99 |
| factor 1 (zinc ion binding) | | | | |
| NP220 nuclear protein (zinc finger) | NM_014497 | 100 | 101 | 102 |
| Heat shock protein 70 | NM_005527 | 103 | 104 | 105 |
| Retinoid binding protein 7 | NM_052960 | 106 | 107 | 108 |
| Regulator of G-protein signaling 2 | NM_002923 | 109 | 110 | 111 |
| Chemokine receptor 2 | NM_000647 | 112 | 113 | 114 |
| Tumor necrosis factor receptor | NM_001243 | 115 | 116 | 117 |
| superfamily, member 8 | | | | |
| Solute carrier family 11 (divalent | NM_000578 | 118 | 119 | 120 |
| metal ion transporter) | | | | |

In some embodiments of the invention, measurement of the upregulation of MT genes or other high risk septic shock genes can be used to separate those patients that are in need of drastic treatment from those patients who are likely to get better with less invasive treatments, such as antibiotic treatment. Many of the currently used septic shock therapies are suitable for high risk patients, but would be unsuitable for lower risk patients who are more likely to improve without drastic measures. For example, pediatric patients with severe septic shock are candidates for cardiopulmonary bypass, but this treatment can be too risky for many patients unless the threat of death is severe.

In some embodiments of the invention, a method of determining whether an individual is at high risk of death due to septic shock is provided, where at least one of the high risk septic shock genes is upregulated. The upregulation can be measured by any suitable means. Examples of measurement techniques include but are not limited to measurement of the presence or level of mRNA, protein, level of post translational modification of a protein, real time PCR, and the like. Preferably, the outcome of the measurement is obtained rapidly, within 24 hours or less, most preferably within about 3 hours, so that suitable therapies can be given immediately. Relatively rapid test measurements, such as dipsticks, test strips, chip technologies, tissue blots, or other methods can be used. The results of these rapid measurements can then be confirmed using additional testing, if desired. An example of the use of a test strip to rapidly detect high risk septic shock in a patient is shown in Example 9.

DNA arrays or gene chips that include one or more of the differentially expressed genes can be used to measure the gene upregulation. An array can also contain a specific subset of the differentially expressed genes that can represent, for example, genes that are only up-regulated in late disease, genes that are only upregulated early in the disease, genes that are only up-regulated in pediatric patients, or genes that are only up-regulated in the presence of certain co-diseases. Protein assays to determine the presence of MT or other signature genes can be performed. An exemplary method of preparing a metallothionein protein assay is shown in Example 6.

Further embodiments of the present invention relate to methods for the diagnosis and analysis of high risk septic shock in a patient. The methods can include, for example, obtaining a patient sample containing mRNA; analyzing gene expression using the mRNA that results in a gene expression signature of that mRNA, wherein the gene expression signature includes the identification and quantification of gene expression from genes that have been identified as being differentially expressed in patients with high risk septic shock; and using that gene expression signature to diagnose or analyze the status of septic shock in the patient, wherein expression of at least about 60% of the signature genes correlates with high risk septic shock. In other embodiments, high risk septic shock is indicated by expression of about 30%, 40%, or 50% or the signature genes, or about 70%, 80%, or 90% of the signature genes.

In additional embodiments of the present invention, a set of genes that is typically downregulated in individuals at high risk of death due to septic shock is provided. Table 3 displays a list of several of these genes. In some embodiments, at least one of the genes that is downregulated in high risk individuals is measured to help in the prediction of risk of death in an individual with septic shock. The expression level of at least about 1, 2, 4, 6, 8, 10, 25, 50, or 100 or more of the set of genes typically downregulated in high risk individuals can be measured, for example, using microarray analysis. The downregulation can be measured by any means known in the art. Examples of measurement techniques include but are not limited to measurement of the presence or level of mRNA, protein, level of post translational modification of a protein, and the like.

The individual to be tested for high risk of death due to septic shock can be of any age. For example, a newborn child, an infant, a toddler, a youth, a teenager, an adult, or an elderly person can be tested. In some embodiments of the invention, any mammal can be tested for high risk of septic shock. Preferably, the mammal is a human.

The individual can be tested, for example, on a one-time bases, then treated accordingly. The individual can be tested periodically, for example to determine whether treatment is progressing. Samples can be taken, for example, about every 30 minutes, every hour, every two hours, every four hours, every 6 hours, every 12 hours, or daily.

The sample to be measured can be taken from various body sources. In some embodiments, the sample is a blood sample. Preferably, a blood sample is taken, the RBCs are separated from the serum, the cells are lysed, and the contents are subjected to the chosen test method. In additional embodiments, a suitable sample can be taken from other cell types or tissues of the body. Additional exemplary sample sources include but are not limited to a tissue, amniotic fluid, urine, bronchoalveolar lavage, and the like.

MT (or other septic shock signature genes of interest) levels can be measured using any suitable method, as known by those of skill in the art. For example, a test for activated MT promoters can be performed, using, for example, PCR methods. A lack of activation of the MT promoters can indicate protection from high risk septic shock.

In additional embodiments, mRNA can be measured. The mRNA can be extracted from a blood sample of the patient, using, for example, a quick prep kit. Procedures such as rtPCR can then be used, in addition to advanced technologies in high density or low density chip format, to quickly and accurately predict whether the patient is at normal risk or high risk of death due to septic shock.

In a further embodiment, MT protein can be measured. MT protein (or other septic shock signature genes of interest) can be measured, for example, using an ELISA or dipstick method. Accordingly, in some embodiments of the invention, kits, assays, dipsticks, and other systems and methods for diagnosing high risk septic shock are provided, by determining the level and variabilities (genetic or protein levels) of high risk septic shock upregulated and downregulated proteins or genes in a patient.

### 400 Signature Genes for High Risk Septic Shock

The microarray analysis used to examine the septic shock signature is described in Examples 4 and 5. The analysis of high risk septic shock patients revealed a set of about 400 differentially expressed genes. These genes, their protein name, accession numbers, cellular information, and other information are listed in Table 1. These septic shock genes can be used for a variety of purposes individually or in various combinations. This set of differentially expressed genes can be thought of as a “signature” or a “fingerprint” of high risk septic shock. The signature can be used, for example, to diagnose high risk septic shock in a patient and to analyze the severity of the disease. In some embodiments of the present invention, the pattern of specifically up- and down-regulated genes is compared to a control, a patient who does not have septic shock, or a patient who has a less severe form of septic shock.

A patient's risk for septic shock-related death can be examined by comparing the patient's expression level of at least one of the signature genes to levels of the signature genes shown in Tables 2-4. However, an exact correlation is not required to be within the scope of the invention. For example, a determination that a patient only exhibits increased expression of some of the signature genes can still be indicative of a patient's risk for death due to septic shock. Thus, a biological sample that is taken from a patient and is determined to have increased expression of, for example, about 1, 5, 10, 20, 30, 40, 50, 60, 70, 80, 90, or 95 percent of the signature genes may still be determined to be at risk of death from septic shock.

The gene expression pattern in combination with the expression level of the gene can be used to indicate an individual's risk for septic shock death. Accordingly, the scope of the invention is not limited to determining whether a patient is at risk for death from septic shock by matching expression levels of all high risk septic shock signature genes. Similarly, it is not required to match the expression levels of all of the signature genes in order to determine that a patient is at risk for death from septic shock. For similar reasons, it is not necessary for a patient's gene expression profile to match exactly the high risk septic shock upregulated and downregulated signature genes in order to determine an individual's prognosis or likely responses to treatment regimes.

In some embodiments of the invention, analysis methods can involve the identification of the signature of differential expression of one or more of the identified genes for a specific patient. In some embodiments, the method includes isolation of mRNA from a diseased tissue, blood sample, or other sample from a patient suspected of having septic shock or exhibiting active septic shock. The expression of the genes that are specifically identified as differentially regulated during high risk septic shock can be analyzed, in comparison to the set of high risk septic shock upregulated and downregulated genes as listed herein. The “signature” is produced as the pattern of up- and down-regulated genes within that patient's sample. The signature can be used for diagnostic methods, for prognostic methods, for analysis of the most efficacious treatment for the patient, and for analysis of the efficacy of the treatment or the progression of the disease.

The gene expression analysis can involve, for example, about 10 genes or more that are identified as differentially expressed in high risk septic shock, preferably at least about 50 genes that are identified as differentially expressed in high risk septic shock, more preferably at least about 100, 200, 300, 400, or 500 genes that are identified as differentially expressed in high risk septic shock, and the like. The genes identified can be expressed at least about 1.1, 1.5, 2, 5, 10, 50, or 100 or more fold higher or lower than normal. Further, in some embodiments, the gene expression of at least about 70% of the genes correlates with that of the gene signature, preferably, the gene expression of at least about 80% of the genes correlates with that of the gene signature, more preferably, the gene expression of at least about 90% of the genes correlates with that of the gene signature, still more preferably, the gene expression of at least about 95% of the genes correlates with that of the gene signature, and the like.

Method of Diagnosis, Prognosis, and Treatment Analysis of a Patient with a High Risk Form of Septic Shock

The genes that are correlated with high risk forms of septic shock can be analyzed as to differential expression in a specific patient by any means known to one of skill in the art. Some embodiments involve isolation of the mRNA from a patient sample.

The isolated mRNA can then be used to analyze gene expression by any method known to one of skill in the art. In one embodiment, the mRNA is used to analyze a “high risk septic shock genechip” or array. From this analysis, a specific patient profile or signature of the genes and amount of differential expression is produced. The amount of differential expression is compared to a normal patient or other control. In some embodiments, the ranges and values of expression for a normal patient are derived using at least 2 normal patients or more, including at least 3, at least 4, at least 5, at least about 10, at least about 20, and at least about 50. In a further embodiment, the ranges and values of expression for a normal patient are derived using a statistical sampling of the population, or a statistical sampling of the area, ethnic group, age group, social group, or sex. In a further embodiment, the range and values of gene expression for a normal patient are derived from the patient before disease or during remission.

The results of the signature can be used in any one or more of the methods disclosed herein. Alternatively, one or more of the analyses can be included in one chip or array. The specific signature can include the results of the expression levels of one or more genes in that specific patient. In one embodiment, the signature is the results of the expression levels of at least about 10 genes, preferably at least about 40 genes, however, the signature can include the results of 50, 60, 70, 80, 90, 100, 150, 200, 250, 500, 750, 1,000, or 2,000 genes that have been identified as being differentially expressed in high risk septic shock. Some genes, such as those in the MT family, are more important or more involved in the manifestation or activation of high risk septic shock. Thus, the signature can require fewer genes when those that are more important have been identified and included.

In one embodiment, the results of the signature are used in a method of diagnosis. The method of diagnosis can include, for example, a method of diagnosis of high risk of death due to septic shock, a method of diagnosis of severity of the disease, a method of diagnosis of a manifestation of the disease and can include any or all of the above.

In another embodiment of the present invention, the results of the high risk septic shock signature can be used for prognosis of the outcome of the disease. The prognosis in various patients can vary tremendously. Some patients can progress to death very rapidly and may need a very aggressive treatment plan. Other patients can have a different reaction and may progress very slowly, requiring a more measured and less aggressive treatment plan. This can be important when considering side effects, quality of life, and patient needs.

In a further embodiment, the results of the septic shock signature are used in methods of identification of the most efficacious treatment for a specific patient. The patient response to a drug or protocol can depend on which genes are being expressed. However, the choice of a treatment method can also involve a number of factors besides the gene expression of specific genes, including, the form of septic shock, the severity of septic shock, the presence of co-diseases, and other patient circumstances. Many of these factors can be identified using one or more of the methods included herein.

### Diagnostic Kits

Additional embodiments of the present invention encompass diagnostic kits to test for high risk septic shock. A kit can be provided, for example, that contains the components for testing an individual for high risk septic shock. The kit can contain, for example, a dipstick assay for measuring the presence of a metallothionein protein, a positive and negative control, instructions, and other materials. The kit can be designed, for example, for use by paramedics, in an emergency room, a hospital room or unit, homecare nursing staff, or home use. In some embodiments, the kits can utilize antibodies that have specific binding affinity to at least one of the proteins produced during high risk septic shock. By “specific binding affinity” is meant that the antibody binds to the target polypeptides with greater affinity than it binds to other polypeptides under specified conditions. Antibodies having specific binding affinity to a septic shock polypeptide can be used in methods for detecting the presence and/or amount of a polypeptide in a sample by contacting the sample with the antibody under conditions such that an immunocomplex forms and detecting the presence and/or amount of the antibody conjugated to the polypeptide. Diagnostic kits for performing such methods can be constructed to include a first container containing the antibody and a second container having a conjugate of a binding partner of the antibody and a label, such as, for example, a radioisotope. The diagnostic kit can also include, for example, notification of an FDA-approved use and instructions.

Preparation of a Microarray for Diagnosis of High Risk of Death from Septic Shock

A microarray device and method to detect high risk septic shock in an individual can be prepared by those of skill in the art. In some embodiments, “array” or “microarray” refers to a predetermined spatial arrangement of capture nucleotide sequences present on a surface of a solid support. The capture nucleotide sequences can be directly attached to the surface, or can be attached to a solid support that is associated with the surface. The array can include one or more “addressable locations,” that is, physical locations that include a known capture nucleotide sequence.

An array can include any number of addressable locations, e.g., 1 to about 100, 100 to about 1000, or more. In addition, the density of the addressable locations on the array can be varied. For example, the density of the addressable locations on a surface can be increased to reduce the necessary surface size. Typically, the array format is a geometrically regular shape, which can facilitate, for example, fabrication, handling, stacking, reagent and sample introduction, detection, and storage. The array can be configured in a row and column format, with regular spacing between each location. Alternatively, the locations can be arranged in groups, randomly, or in any other pattern. In some embodiments an array includes a plurality of addressable locations configured so that each location is spatially addressable for high-throughput handling. Examples of arrays that can be used in the invention have been described in, for example, U.S. Pat. No. 5,837,832, which is hereby incorporated by reference in its entirety.

In a two-dimensional array the addressable location is determined by location on the surface. However, in some embodiments the array includes a number of particles, such as beads, in solution. Each particle includes a specific type or types of capture nucleotide sequence(s). In this case the identity of the capture nucleotide sequence(s) can be determined by the characteristics of the particle. For example, the particle can have an identifying characteristic, such as shape, pattern, chromophore, or fluorophore.

Depending upon the type of array used in various embodiments according to the present invention, the methods of detecting hybridization between a capture nucleotide sequence and a target nucleic acid sequence can vary. For example, target nucleotide sequences can be labeled before application to the microarray. Through hybridization of the target sequence to the capture probe of complementary sequence on the array, the label is bound to the array at a specific location, revealing its identity. Use of glass substrates for microarray design has permitted the use of fluorescent labels for tagging target sequences. Fluorescent labels are particularly useful in microarray designs that employ glass beads as a solid support for the array; these beads can be interrogated using fiber optics and the measurement of the presence and strength of a signal can be automated (Ferguson, J A et al. (1996) Nat Biotechnol 14:1681-1684, which is hereby incorporated by reference in its entirety). Labeling of target DNA with biotin and detection of the hybridized target on the array with antibodies to biotin is an alternative approach that is within the level of skill in the art (Cutler, D J), which is incorporated herein by reference in its entirety.)

The terms “polynucleotide” and “oligonucleotide” are used in some contexts interchangeably to describe single-stranded and double-stranded polymers of nucleotide monomers, including 2′-deoxyribonucleotides (DNA) and ribonucleotides (RNA). A polynucleotide can be composed entirely of deoxyribonucleotides, entirely of ribonucleotides, or chimeric mixtures thereof. Likewise polynucleotides can be composed of, for example, internucleotide, nucleobase and sugar analogs, including unnatural bases, sugars, L-DNA and modified internucleotide linkages. The capture nucleotide sequencers) of the invention fall within this scope and in preferred embodiments the term “primer(s)” is used interchangeably with capture nucleotide sequence(s). “Target nucleotide sequence” refers in preferred embodiments to a specific candidate gene, the presence or absence of which is to be detected, and that is capable of interacting with a capture nucleotide sequence.

The term “capture” generally refers to the specific association of two or more molecules, objects or substances which have affinity for each other. In specific embodiments of the present invention, “capture” refers to a nucleotide sequence that is present for its ability to associate with another nucleotide sequence, typically from a sample, in order to detect or assay for the sample nucleotide sequence.

Typically, the capture nucleotide sequence has sufficient complementarity to a target nucleotide sequence to enable it to hybridize under selected stringent hybridization conditions, and the Tm is generally about 10° to 20° C. above room temperature (e.g., in many cases about 37° C.). In general, a capture nucleotide sequence can range from about 8 to about 50 nucleotides in length, preferably about 15, 20, 25 or 30 nucleotides. As used herein, “high stringent hybridization conditions” means any conditions in which hybridization will occur when there is at least 95%, preferably about 97 to 100%, nucleotide complementarity (identity) between the nucleic acids. In some embodiments, modifications can be made in the hybridization conditions in order to provide for less complementarity, e.g., about 90%, 85%, 75%, 50%, etc.

The choice of hybridization reaction parameters to be used will be within the scope of those in their art. The parameters, such as salt concentration, buffer, pH, temperature, time of incubation, amount and type of denaturant such as formamide, etc. can be varied as desired (See, e.g., Sambrook et al. (1989) Molecular Cloning: A Laboratory Manual (2d ed.) Vols. 1-3, Cold Spring Harbor Press, New York; Hames et al (1985) Nucleic Acid Hybridization IL Press; Davis et al. (1986) Basic Methods in Molecular Biology, Elsevier Sciences Publishing, Inc., New York; each one of which is hereby incorporated by reference in its entirety.) For example, nucleic acid (e.g., linker oligonucleotides) can be added to a test region (e.g., a well of a multiwell plate—in a preferred embodiment, a 96 or 384 or greater well plate), in a volume ranging from about 0.1 to about 100 or more μl (in a preferred embodiment, about 1 to about 50 μl, most preferably about 40 μl), at a concentration ranging from about 0.01 to about 5 μM (in a preferred embodiment, about 0.1 μM), in a buffer such as, for example, 6×SSPE-T (0.9 M NaCl, 60 mM NaH₂ PO₄, 6 mM EDTA and 0.05% Triton X-100), and hybridized to a binding partner (e.g., a capture nucleotide sequence on the surface) for between about 10 minutes and about at least 3 hours. In a preferred embodiment, the hybridization takes place for at least about 15 minutes. The temperature for hybridization can range, for example from about 4° C. to about 37° C. In a preferred embodiment, the temperature is about room temperature.

In general, the term “solid support” can refer to any solid phase material upon which a capture nucleotide sequence can be attached or immobilized. For example, a solid support can include glass, metal, silicon, germanium, GaAs, plastic, or the like. In some embodiments, a solid support can refer to a “resin,” “solid phase,” or “support.” A solid support can be composed, for example, of organic polymers such as polystyrene, polyethylene, polypropylene, polyfluoroethylene, polyethyleneoxy, and polyacrylamide, as well as co-polymers and grafts thereof, and the like. A solid support can also be inorganic, such as glass, silica, controlled-pore-glass (CPG), reverse-phase silica, and the like. The configuration of a solid support can be in the form of beads, spheres, particles, granules, a gel, a fiber or a surface. Surfaces can be, for example, planar, substantially planar, or non-planar. Solid supports can be porous or non-porous, and can have swelling or non-swelling characteristics. A solid support can be configured in the form of a well, depression or other container, slide, plate, vessel, feature or location. In some embodiments, a plurality of solid supports can be configured in an array.

Capture nucleotide sequences can be synthesized by any suitable means. The synthesis can occur, for example, by conventional technology, e.g., with a commercial oligonucleotide synthesizer and/or by ligating together subfragments that have been so synthesized. For example, preformed capture nucleotide sequences, can be situated on or within the surface of a test region by any of a variety of conventional techniques, including photolithographic or silkscreen chemical attachment, disposition by ink jet technology, electrochemical patterning using electrode arrays, or denaturation followed by baking or UV-irradiating onto filters (see, e.g., Rava et al. (1996) U.S. Pat. No. 5,545,531; Fodor et al. (1996) U.S. Pat. No. 5,510,270; Zanzucchi et al. (1997) U.S. Pat. No. 5,643,738; Brennan (1995) U.S. Pat. No. 5,474,796; PCT WO 92/10092; PCT WO 90115070; each one of which is hereby incorporated by reference in its entirety).

### Treatment of Septic Shock

In further embodiments of the invention, methods of treatment of an individual at high risk for death from septic shock are provided. For example, some embodiments of the invention provide a treatment for high risk septic shock by administration of a compound that modulates MT expression, protein production, or protein function. Such treatments can include, for example, administering molecules that downregulate MT expression, or administering molecules that downregulate the expression of other high risk septic shock-related genes. Other treatments can include, for example, administering compositions that are capable of upregulating at least one of the beneficial genes that is typically downregulated in high risk septic shock individuals.

As used herein, the term “treat” or “treatment” can refer to both therapeutic treatment and prophylactic or preventative measures, wherein the object is to prevent or reduce or reverse the progression of septic shock in an individual. In some embodiments, the treatment can prevent septic shock-induced death of the individual. The term “treat” can also refer to the characterization of the type or severity of disease which can have ramifications for future prognosis, or need for specific treatments. For purposes of this invention, beneficial or desired clinical results can include, but are not limited to, alleviation of septic shock symptoms, diminution of extent of septic shock, reduced risk of death from septic shock, stabilized (such as being characterized by not worsening) state of septic shock, delay or slowing of septic shock progression, amelioration or palliation of a septic shock-induced state, and remission (whether partial or total), whether detectable or undetectable. The term “treatment” can also encompass prolonging survival as compared to expected survival if not receiving treatment. Those in need of treatment include, for example, those already diagnosed with septic shock, as well as those prone to have septic shock, those of high risk of death due to septic shock, and those in which septic shock is to be prevented.

### Zinc and MT

Many of the genes found to be downregulated in the high risk septic shock group are zinc-dependent factors. For example, many MT genes are activated by zinc-requiring transcription factors. Once zinc is available, the transcription factor can bind to the MT promoters, thus allowing MT expression. Because MT binds to Zn and other metals, the MT proteins, once produced, can bind to and even sequester zinc, often causing a zinc-starved state. This Zn starvation in an individual can lead to many types of diseases. Thus, in some embodiments of the present invention, providing zinc to a patient can allow the expression of many of these “beneficial” genes and can ameliorate other effects of Zn starvation, permitting the individual to better respond to the septic shock episode.

Accordingly, in some embodiments of the present invention, zinc supplementation or zinc replacement can be used to treat septic shock, by inducing the upregulation of several genes that are typically downregulated during severe septic shock. The zinc to be administered can take any suitable form, and can be administered, for example, orally, intravenously, by injection, or by other suitable methods. The zinc can be combined with other compounds, such as other metals, vitamins, solubilizing agents, salt forms, and the like. Intravenous administration is generally preferred. Example 11 demonstrates the use of intravenous zinc administration to treat high risk septic shock.

Accordingly, individuals with high risk septic shock have been found to have lower levels of zinc in serum samples, as shown in FIG. 5. In additional embodiments of the invention, screening individuals for zinc levels in the blood can be used to determine individuals at higher risk for death from septic shock. Thus, in some embodiments, a diagnosis can involve a simple test for free or bound zinc in a blood or tissue sample. Zinc quantitation is typically measured by atomic absorption. An example of testing a patient for serum zinc levels is shown in Example 10.

### Identification of Drug Targets for Septic Shock Treatment

The high risk septic shock signature genes can also be utilized to identify septic shock drug targets. Any or all of the genes identified herein and included in the signature or on a septic shock array can be used to further identify drugs or treatments that can target a desired gene or gene product. Preferred drugs and treatments include those that can downregulate deleterious genes and/or their products such as, for example, the MT genes and MT proteins; likewise, drugs and treatments that can activate or enhance expression of protective genes and/or their products are also among preferred embodiments of the invention. Methods of identifying targets can include any method known to those of skill in the art, including, but not limited to: producing and testing small molecules, oligonucleotides (including antisense, RNAi, molecular decoy methods, and triplex formers), antibodies, and drugs that target any of the genes or gene products identified herein. Gene therapy approaches can also be used to down-regulate, up-regulate, or express proteins or gene products identified herein.

### Administration of a Vector Having an Antisense MT Sequence

In additional embodiments of the invention, an antisense MT nucleic acid is provided that can be delivered to a host cell via any suitable method, such as injection into a tissue, electroporation to an in vitro cell culture, or other methods. This approach can be used, for example, to develop in vitro or animal models of molecular, cellular, or physiological events associated with high risk septic shock. Example 12 demonstrates the use of this method to treat septic shock. Nucleic acids can be delivered, for example, as naked DNA or within vectors, the vectors including, but not limited to viral, plasmid, cosmid, liposome, and microparticles. The individual or host cell can then be tested to determine if the antisense MT sequence causes downregulation of one or more MT genes, and if the severity of septic shock decreases over time. A similar method can be used for other septic shock upregulated genes.

### EXAMPLES

The following examples are offered to illustrate, but not to limit, the claimed invention.

### Example 1

### Database of Septic Shock Pediatric Patients

To determine whether molecular differences can predict those patients that survived septic shock conditions versus those that would succumb, a database of normal and critically ill pediatric patients was assembled and examined. The database contained 60 different samples from 13 normal individuals and 32 critically ill patients, 15 of whom contributed two samples. A first sample was taken on the first day of admission to the critical care or intensive care unit. A second sample was taken on the third day of the patient's stay. The databases included data relating to blood counts, infecting organisms, patient survival, and other diagnostic factors. Details of the condition of each patient are shown below in Table 5.

TABLE 5A
Patient and Clinical Information

| | | | Sample | | | | Total | | |
|---|---|---|---|---|---|---|---|---|---|
| Patient | | | Collection | Sample | | | WBC | | |
| ID | Diagnosis | survival | Day | Number | steroid | PRISM | (X100) | % Segs | % Bands |
| 01_0013 | SepticShock | nonsurvivor | 1 | 0 | − | n/a | n/a | n/a | n/a |
| 11_0017 | SepticShock | nonsurvivor | 1 | 36 | + | n/a | 7.2 | 30 | 19 |
| 11_0017 | SepticShock | nonsurvivor | 3 | 37 | + | n/a | 3.4 | n/a | n/a |
| 26_260611 | SepticShock | nonsurvivor | 1 | 54 | + | 59 | 1 | 10 | 12 |
| 04_0005 | SepticShock | nonsurvivor | 1 | 4 | + | 20 | 19.4 | 86.4 | 0 |
| 10_0017 | SepticShock | nonsurvivor | 1 | 23 | − | 9 | 11.1 | 82 | 3 |
| 10_0017 | SepticShock | nonsurvivor | 3 | 24 | − | 9 | 18.1 | 76 | 16 |
| 06_0003 | SepticShock | survivor | 1 | 12 | − | n/a | n/a | n/a | n/a |
| 06_0003 | SepticShock | survivor | 3 | 13 | − | n/a | n/a | n/a | n/a |
| 01_0022 | SepticShock | survivor | 3 | 50 | − | n/a | 7.1 | 22 | 33 |
| 10_0012 | SepticShock | survivor | 1 | 19 | − | 25 | 15.3 | 48 | 23 |
| 09_0001 | SepticShock | survivor | 1 | 16 | − | 22 | 4.5 | 61 | 0 |
| 10_0001 | SepticShock | survivor | 1 | 18 | − | 22 | 26 | 72 | 5 |
| 05_0007 | SepticShock | survivor | 1 | 58 | + | 22 | 3.1 | 69 | 12 |
| 05_0007 | SepticShock | survivor | 3 | 59 | + | 22 | 22.6 | 82 | 8 |
| 01_0014 | SepticShock | survivor | 1 | 1 | − | 20 | 13.4 | 41 | 5 |
| 06_0001 | SepticShock | survivor | 1 | 9 | + | 18 | 7 | 71 | 20 |
| 04_0002 | SepticShock | survivor | 3 | 3 | + | 16 | 13.6 | n/a | n/a |
| 27_70603 | SepticShock | survivor | 1 | 55 | − | 15 | 18.4 | n/a | n/a |
| 05_0006 | SepticShock | survivor | 1 | 7 | + | 12 | 9 | 54 | 2 |
| 12_0001 | SepticShock | survivor | 1 | 60 | − | 6 | 44.1 | 51 | 36 |
| 01_0021 | SepticShock | survivor | 1 | 2 | − | 5 | 28.5 | 76 | 0 |
| 06_0002 | SIRS | survivor | 1 | 11 | − | n/a | n/a | n/a | n/a |
| 11_0004 | SIRS | survivor | 1 | 25 | − | n/a | 13.4 | n/a | n/a |
| 11_0015 | SIRS | survivor | 1 | 32 | − | n/a | 12.3 | n/a | n/a |
| 11_0015 | SIRS | survivor | 3 | 33 | − | n/a | 8.4 | n/a | n/a |
| 11_0016 | SIRS | survivor | 1 | 34 | − | n/a | n/a | n/a | n/a |
| 11_0021 | SIRS | survivor | 1 | 41 | − | n/a | n/a | n/a | n/a |
| 11_0006 | SIRS | survivor | 1 | 44 | + | n/a | 19.2 | 79 | 0 |
| 25_70603 | SIRS | survivor | 3 | 53 | − | n/a | n/a | n/a | n/a |
| 10_0002 | SIRS | survivor | 1 | 56 | − | 28 | 7.4 | 53 | 3 |
| 10_0002 | SIRS | survivor | 3 | 57 | − | 28 | 3.9 | 33.2 | 0 |
| 10_0012 | SIRS | survivor | 3 | 20 | − | 25 | 10.1 | 64 | 19 |
| 09_0001 | SIRS | survivor | 3 | 17 | − | 22 | 7.2 | 63 | 17 |
| 10_0013 | SIRS | survivor | 3 | 21 | − | 11 | 8.7 | 82 | 7 |
| 04_0004 | SIRS | survivor | 1 | 51 | + | 11 | 22.8 | 76 | 0 |
| 04_0004 | SIRS | survivor | 3 | 52 | + | 11 | 11.8 | n/a | n/a |
| 10_0015 | SIRS | survivor | 1 | 22 | − | 6 | 9.2 | 92 | 0 |
| 07_0005 | SIRS | survivor | 1 | 14 | − | 4 | 15.2 | 52 | 11 |
| 07_0005 | SIRS | survivor | 3 | 15 | − | 4 | 13.3 | 67 | 0 |
| 05_0002 | SIRS | survivor | 1 | 5 | + | 2 | 13.2 | 91 | 0 |
| 11_0016 | SIRS_resolved | survivor | 3 | 35 | − | n/a | 10.1 | n/a | n/a |
| 11_0021 | SIRS_resolved | survivor | 3 | 42 | − | n/a | n/a | n/a | n/a |
| 11_0006 | SIRS_resolved | survivor | 3 | 45 | + | n/a | 18.5 | 35 | 35 |
| 10_0001 | SIRS_resolved | survivor | 3 | 43 | − | 22 | 26 | 72 | 5 |
| 06_0001 | SIRS_resolved | survivor | 3 | 10 | + | 18 | 11.1 | 68 | 11 |
| 05_0006 | SIRS_resolved | survivor | 3 | 8 | + | 12 | 9.3 | 76 | 0 |
| 11_0008 | Control | survivor | 1 | 26 | ctl | n/a | n/a | n/a | n/a |
| 11_0009 | Control | survivor | 1 | 27 | ctl | n/a | n/a | n/a | n/a |
| 11_0011 | Control | survivor | 1 | 28 | ctl | n/a | n/a | n/a | n/a |
| 11_0012 | Control | survivor | 1 | 29 | ctl | n/a | n/a | n/a | n/a |
| 11_0013 | Control | survivor | 1 | 30 | ctl | n/a | n/a | n/a | n/a |
| 11_0014 | Control | survivor | 1 | 31 | ctl | n/a | n/a | n/a | n/a |
| 11_0018 | Control | survivor | 1 | 38 | ctl | n/a | n/a | n/a | n/a |
| 11_0019 | Control | survivor | 1 | 39 | ctl | n/a | n/a | n/a | n/a |
| 11_0020 | Control | survivor | 1 | 40 | ctl | n/a | n/a | n/a | n/a |
| 15_0001 | Control | survivor | 1 | 46 | ctl | n/a | n/a | n/a | n/a |
| 15_0002 | Control | survivor | 1 | 47 | ctl | n/a | n/a | n/a | n/a |
| 15_0003 | Control | survivor | 1 | 48 | ctl | n/a | n/a | n/a | n/a |
| 15_0005 | Control | survivor | 1 | 49 | ctl | n/a | n/a | n/a | n/a |

TABLE 5B
Patient and Clinical Information

| Patient | | % | | | | Organism | Infect. |
|---|---|---|---|---|---|---|---|
| ID | % Lymphocytes | Monocytes | Sample # | Steroid | Organism | Class | Site |
| 01_0013 | n/a | n/a | 0 | − | none | none | none |
| 11_0017 | 45 | 6 | 36 | + | none | none | none |
| 11_0017 | n/a | n/a | 37 | + | none | none | none |
| 26_260611 | 70 | 0 | 54 | + | N. meningitidis | gram neg | Blood |
| 04_0005 | 10.1 | 3 | 4 | + | Group A Strep | gram pos | Blood |
| 10_0017 | 11 | 2 | 23 | − | Staph Epi | gram pos | wound infect w |
| | | | | | | | blood |
| 10_0017 | 5 | 1 | 24 | − | Staph Epi | gram pos | wound infect w |
| | | | | | | | blood |
| 06_0003 | n/a | n/a | 12 | − | none | none | none |
| 06_0003 | n/a | n/a | 13 | − | none | none | none |
| 01_0022 | 29 | 12 | 50 | − | none | none | none |
| 10_0012 | 10 | 10 | 19 | − | E coli | gram neg | Blood |
| 09_0001 | 25 | 14 | 16 | − | none | none | none |
| 10_0001 | 15 | 8 | 18 | − | mult gram neg | gram neg | Blood |
| 05_0007 | 16 | 3 | 58 | + | Group A Strep | gram pos | Blood |
| 05_0007 | 5 | 0 | 59 | + | Group A Strep | gram pos | Blood |
| 01_0014 | 40 | 10 | 1 | − | Candida albicans | fungal | Lung |
| 06_0001 | 4 | 5 | 9 | + | mult gram neg | gram neg | Blood |
| 04_0002 | n/a | n/a | 3 | + | E. coli (HUS) | gram neg | Blood |
| 27_70603 | n/a | n/a | 55 | − | none | none | none |
| 05_0006 | 33 | 11 | 7 | + | none | none | none |
| 12_0001 | 7 | 6 | 60 | − | Strep Pneum | gram pos | Blood |
| 01_0021 | 16 | 8 | 2 | − | none | none | none |
| 06_0002 | n/a | n/a | 11 | − | none | none | none |
| 11_0004 | n/a | n/a | 25 | − | none | none | none |
| 11_0015 | n/a | n/a | 32 | − | none | none | none |
| 11_0015 | n/a | n/a | 33 | − | none | none | none |
| 11_0016 | n/a | n/a | 34 | − | none | none | none |
| 11_0021 | n/a | n/a | 41 | − | none | none | none |
| 11_0006 | 11 | 10 | 44 | + | none | none | none |
| 25_70603 | n/a | n/a | 53 | − | none | none | none |
| 10_0002 | 30 | 14 | 56 | − | none | none | none |
| 10_0002 | 51 | 15.4 | 57 | − | none | none | none |
| 10_0012 | 5 | 12 | 20 | − | E coli | gram neg | Blood |
| 09_0001 | 13 | 4 | 17 | − | none | none | none |
| 10_0013 | 4 | 4 | 21 | − | none | none | none |
| 04_0004 | 11 | 13 | 51 | + | none | none | none |
| 04_0004 | n/a | n/a | 52 | + | none | none | none |
| 10_0015 | 7 | 0 | 22 | − | none | none | none |
| 07_0005 | 25 | 10 | 14 | − | none | none | none |
| 07_0005 | 26 | 5 | 15 | − | none | none | none |
| 05_0002 | 6 | 3 | 5 | + | none | none | none |
| 11_0016 | n/a | n/a | 35 | − | none | none | none |
| 11_0021 | n/a | n/a | 42 | − | none | none | none |
| 11_0006 | 11 | 14 | 45 | + | none | none | none |
| 10_0001 | 15 | 8 | 43 | − | none | none | none |
| 06_0001 | 13 | 8 | 10 | + | mult gram neg | gram neg | Blood |
| 05_0006 | 18 | 6 | 8 | + | none | none | none |
| 11_0008 | n/a | n/a | 26 | ctl | none | none | none |
| 11_0009 | n/a | n/a | 27 | ctl | none | none | none |
| 11_0011 | n/a | n/a | 28 | ctl | none | none | none |
| 11_0012 | n/a | n/a | 29 | ctl | none | none | none |
| 11_0013 | n/a | n/a | 30 | ctl | none | none | none |
| 11_0014 | n/a | n/a | 31 | ctl | none | none | none |
| 11_0018 | n/a | n/a | 38 | ctl | none | none | none |
| 11_0019 | n/a | n/a | 39 | ctl | none | none | none |
| 11_0020 | n/a | n/a | 40 | ctl | none | none | none |
| 15_0001 | n/a | n/a | 46 | ctl | none | none | none |
| 15_0002 | n/a | n/a | 47 | ctl | none | none | none |
| 15_0003 | n/a | n/a | 48 | ctl | none | none | none |
| 15_0005 | n/a | n/a | 49 | ctl | none | none | none |

### Example 2

### Preparation of Samples for Microrarray Analysis

Patient blood samples taken from the individuals described in Example 1 were used to measure gene expression using the following microarray diagnostic procedure. Whole blood was collected into PaxGene blood RNA system preparation tubes and RNA was prepared according to manufacturer's directions (Qiagen Inc., Valencia, Calif.). The purified RNA quality was validated using an Agilent 2100 Bioanalyzer (Agilent, Palo Alto, Calif.). Labeling was performed using standard protocols from Affymetrix. The labeled material was hybridized to an Affymetrix GeneChip 133plus2 (Affymetrix, Santa Clara, Calif.). The results of the GeneChip read-out were analyzed and subjected to data analysis procedures.

### Example 3

### Additional Analysis of Septic Shock Patients

Additional analyses of septic shock patient samples can be performed, if desired, in addition to the microarray analysis procedure. Examples include blood cultures, complete blood count, invading organism determination, serum zinc levels, and cellular MT levels. Additional assays can be performed, for example, to determine the degree of organ failure, or the presence of other diseases in the patient. The additional assays can also be performed to confirm the septic shock diagnosis and to provide other information on the patient health status. Additional materials that can be characterized for this predictive diagnostic procedure include DNA isolated from whole blood, serum and plasma isolated from whole blood, other non-blood tissue samples, saliva, urine, and respiratory exhalation.

### Example 4

### Microarray Analysis Method for Determination of Expression Profiles

The initial microarray data (Affymetrix CEL files) was subjected to an RMA normalization procedure. This procedure decreases processing related variation in expression to normalize each chip to its median value, then to each probe set to differences that occur across all chips in the group. Each measurement was divided by the 50.0^{th} percentile of all measurements in that sample. Specific samples were normalized to one another: sample(s) 1-60 were normalized against the median of the control sample(s). Each measurement for each gene in those specific samples was divided by the median of that gene's measurements in the corresponding control samples. Gene expression values were thus depicted relative to the level of expression in the control sample.

### Example 5

### Results of Microarray Analysis of Septic Shock Patients

In order to evaluate the relative statistical strength of various genes to predict those children at risk for death, statistical tests were performed. Genes were identified that were overexpressed or underexpressed in the nonsurviving children as compared to children that did survive. The comparison group of nonsurvivors can be chosen from either all children with a similar presenting condition, or from similar plus dissimilar presenting illness children that do not die. In this case a pool of genes was derived from two procedures as described below. The two procedures are identical, except that different statistical tests were performed. The gene lists generated by each of these tests were then pooled to generate the final list of 400 genes.

### Procedure 1:

Several key genes were identified from among all genes with statistically significant differences between the following groups based on values of ‘survival’ and ‘SepsSirsDx’: survivor, SepSir, versus nonsurvivor, SepSir using a parametric test with variances assumed equal (Student's t-test). The p-value cutoff was 0.05, and multiple testing correction used the Benjamini and Hochberg False Discovery Rate. This restriction tested 54,681 genes; 6 genes had insufficient data for a comparison. About 5.0% of the identified genes would be expected to pass the restriction by chance. This led to the detection of 133 genes, of which 9 of the 30 genes with the lowest p-value are metallothionein genes.

### Procedure 2:

Key genes were identified from among all genes with statistically significant differences between the following groups based on values of ‘survival’ and ‘SepsSirsDx’: survivor, SepSir, versus nonsurvivor, SepSir using a parametric test with variances not assumed to be equal (Welch t-test). The p-value cutoff was 0.05, and multiple testing correction used the Benjamini and Hochberg False Discovery Rate. This restriction tested 54,681 genes; 6 genes had insufficient data for a comparison. About 5.0% of the identified genes would be expected to pass the restriction by chance. This led to the detection of 278 genes, of which the majority were overexpressed in the children that did not die, and were underexpressed in children that did die.

The combination of the two above-described gene lists led to a list of 400 genes (only 11 genes in common). The relative power of the two lists to strongly separate the patients that die from those that did not die was unexpectedly high.

Two methods enabled the ability to use this pool of 400 genes to distinguish, and thus to form a prediction of the children that would die from those that would survive. The first method was a hierarchical clustering method that used Euclidean distance and the Standard correlation as the distance metrics to arrange genes and patients in groups or clusters in which patients are essentially categorized and genes are categorized that shared similar expression across the group of all patients. Two principle patterns were evident in this analysis: genes that were overexpressed in the children that would die and those that were induced in children that would not die, but are not as induced in the children that would die. This model suggests an advantage for children to induce those “protective” genes and that experimental therapies that decreased the induction or effects of the protective genes would fail to have a positive impact. Conversely, the effects of genes that are induced in the most significant fashion in the patients that die can be harmful and therapies that diminish the extent of the induction or the effects of this induction can be helpful.

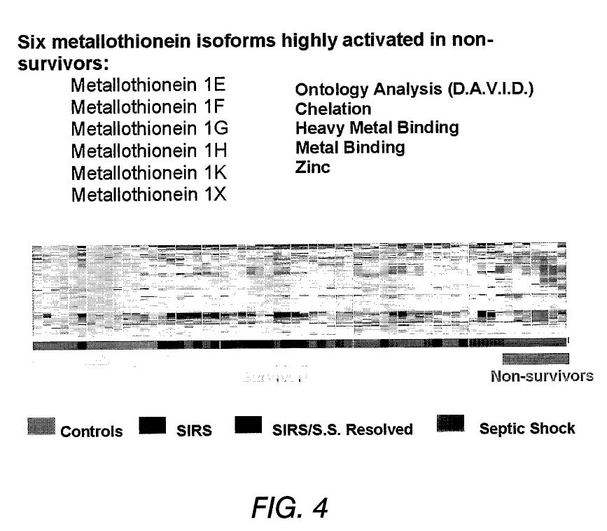

The 400 genes found to be predictors of non-survival is shown in FIG. 1. Tables 1-3 list selected genes that are either upregulated or repressed/downregulated in the non-survivors. FIG. 4 shows the gene expression signature of six of the metallothionein family members that were activated during septic shock in the non-survivors.

### Example 6

### Preparation of a Metallothionein Protein Assay

The following method can be used to prepare an assay for the presence and quantitation of metallothionein in a patient sample. A metallothionein protein of interest is isolated and purified. The isolated protein is injected into rabbits to produce polyclonal antibodies using methods well known by those of skill in the art. The antibodies are collected, purified, and tested. The antibodies are used to prepare an assay to determine the presence of metallothionein in a blood sample. The sample is prepared by collecting blood from the patient, separating the cells from the serum, and lysing the cells. The assay is used to determine, qualitatively or quantitatively, the presence or absence of the metallothionein protein. Positive and negative controls are used to confirm the accuracy of the test method.

### Example 7

### Metallothionein as a Biomarker for High-Risk Septic Shock

A blood sample is taken from a one year old hospitalized child exhibiting symptoms of septic shock. The blood sample is assayed for the presence of the metallothionein protein. Within two hours, the test results are available, showing that the individual tests positive for the high risk metallothionein marker protein. Using this information, the pediatrician immediately puts in place emergency life-saving procedures such as for example, zinc treatment and/or cardiopulmonary bypass, in addition to the usual septic shock treatment procedures.

### Example 8

### High Risk Septic Shock Markers are Used to Confirm the Diagnosis of High-Risk Septic Shock in a Pediatric Patient

A blood sample is taken from the one year old hospitalized child discussed in Example 7. To confirm the metallothionein marker test of high risk probability, a microarray assay is performed. A commercially prepared gene chip having a set of 25 high risk septic shock upregulated genes, and a set of 20 high risk septic shock down-regulated genes, is obtained. mRNA is isolated from the blood sample using methods well known in the art, and the sample is tested for the presence of the indicated genes. Using this method, the individual described in Example 7 above is confirmed as having a high risk of death from septic shock. With this knowledge, treatment of high risk septic shock by extracorporeal membrane oxygenation and plasmapheresis is initiated. Additional therapies directed toward shutting down MT genes and replacing zinc are administered. By use of the fast diagnosis and treatment program, the patient survives.

### Example 9

### Test Strip Kit for Early and Fast Detection of Septic Shock in a Clinical Environment

A commercial test kit for septic shock is prepared, using antibodies to the human metallothionein protein. The antibodies are used to prepare a commercial dipstick assay kit for determining the presence of a metallothionein family protein in a blood sample of a patient, using assay preparation methods well known by those of skill in the art. The assay also includes positive and negative controls. Using this assay, the practitioner can quickly determine whether an individual is at high risk for death due to septic shock.

### Example 10

### Measurement of Serum Zinc Levels in Survivors vs. Non-Survivors

To determine the relationship between zinc levels and survivorship, levels of zinc in the patient serum samples was determined. The non-survivors had about 500 μg/liter of zinc, which was less than half of the serum zinc level (about 1.1 mg/liter) found to be present in the septic shock survivor group (FIG. 5). This result demonstrates that zinc levels may be low in the non-surviving group of septic shock individuals.

### Example 11

### Administration of an Intravenous Zinc Formulation to Treat High Risk Septic Shock

A severely ill patient with a high risk of developing septic shock due to illness complications is identified. The patient is administered a daily mineral supplement containing zinc in an intravenous form. By use of this method, the patient's health improves, and the likelihood that the patient will develop high risk septic shock is reduced.

### Example 12

### Treatment of High Risk Septic Shock with Nucleic Acids that Downregulate MT Expression

An individual with septic shock tests positive for several septic shock high risk markers. The individual is treated by intravenous injection with a vector having an MT antisense nucleic acid. Using this method, MT protein level decreases within approximately eight hours, and the patient's health improves.

All references cited herein, including patents, patent applications, papers, text books, and the like, and the references cited therein, to the extent that they are not already, are hereby incorporated herein by reference in their entirety.

The foregoing description and examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. An assay to determine the potential of high risk septic shock in an individual, comprising:
obtaining a biological sample from the individual; and
determining a level of expression of at least one septic shock signature gene;
where an increased level of expression of the at least one septic shock signature gene indicates an elevated risk of death from septic shock.

2. The assay of claim 1, wherein said signature gene encodes a protein chosen from the group consisting of: a metallothionein protein, Metallothionein 1E, Metallothionein 1F, Metallothionein 1G, Metallothionein 1H, Metallothionein 1K, Metallothionein 1X, Granzyme B (cytotoxic serine protease), Dual specific phosphatase 2 (inactivation of MAPK), Regulator of G-protein signaling 1, v-Jun & Jun dimerization protein, Chemokine ligand 2 (MCP-1), Chemokine ligand 3 (MIP-1α), Chemokine (C—C motif) receptor-like 2, cAMP responsive element modulator, Complement factor H, SOCS1, Interferon-γ, and Interferon regulatory factor 7.

3. The assay of claims 1 or 2, wherein said individual is a mammal.

4. The assay of claim 3, wherein said mammal is a human.

5. The assay of claim 4, wherein said human is selected from the group consisting of: an elderly person, an adult, a child, an infant, a newborn, and an unborn child.

6. The assay of claims 1 or 2, wherein said sample is selected from the group consisting of: a blood sample, a tissue sample, an amniotic fluid sample, a urine sample, and a bronchoalveolar lavage sample.

7. A test kit for the early identification of high risk septic shock, comprising two or more nucleic acid sequences adapted for indicating presence of absence of at least one septic shock signature gene in a biological sample.

8. The test kit of claim 7, wherein said kit comprises a probe that determines the presence of metallothionein mRNA or protein in a sample.

9. The test kit of claim 8, further comprising at least one component selected from the group consisting of: an instruction sheet, a sample collection device, a sample preparation device, positive controls, and negative controls.

10. A method of treating an individual having septic shock, comprising administering a metallothionein-reducing agent.

11. A method of treating an individual having septic shock, comprising administering an agent that downregulates at least one gene listed in tables 2 and 3.

12. A method of treating septic shock in an individual, comprising administering an agent that upregulates at least one of the genes listed in table 4.

13. A method of treating septic shock in an individual, comprising administering zinc.

14. The method of claim 13, wherein said zinc is in at least one form selected from the group consisting of: zinc sulfate, zinc gluconate, and zinc chloride.

15. The method of claim 13, wherein said zinc is administered intravenously.

16. A method of identifying an individual at high risk of death from septic shock, comprising:
identifying an individual that may have septic shock;
obtaining a blood or other bodily sample from said individual;
testing said sample for at least one of septic shock signature genes; and
determining an altered signature gene profile as compared to control samples, thereby determining that an elevated risk of death from septic shock exists in said individual.

17. The method of claim 16, wherein at least 5 septic shock signature genes are tested.

18. The method of claims 16 or 17, wherein said control samples are obtained from individuals with septic shock who were able to survive the episode.

19. The method of claims 16, 17 or 18, wherein said testing is performed by microarray analysis or a dipstick assay.
